SEQUENCE LISTING (257 sequences)

SEQ ID NO: 1 - protein, 313 aa, synthetic construct
  REGION 1..313: note = ASPA mutation
MTSCHIAEEHIQKVAIFGGTHGNELTGVFLVKHWLENGAEIQRTGLEVKPFITNPRAVKKCTRYIDCDLNRIFDLENLGKKMSEDLPYEVRRAQEINHLFGPKDSEDSYDIIFDLHNTTSNMGCTLILEDSRNNFLIQMFHYIKTSLAPLPCYVYLIEHPSLKYATTRSIAKYPVGIEVGPQPQGVLRADILDQMRKMIKHALDFIHHFNEGKEFPPCAIEVYKIIEKVDYPRDENGEIAAIIHPNLQDQDWKPLHPGDPMFLTLDGKTIPLGGDCTVYPVFVNEAAYYEKKEAFAKTTKLTLNAKSIRCCLH

SEQ ID NO: 2 - DNA, 942 bp, synthetic construct
  misc_feature 1..942: note = hASPA-R132G
  source 1..942: mol_type = other DNA
atgacttcttgtcacattgctgaagaacatatacaaaaggttgctatctttggaggaacccatgggaatgagctaaccggagtatttctggttaagcattggctagagaatggcgctgagattcagagaacagggctggaggtaaaaccatttattactaaccccagagcagtgaagaagtgtaccagatatattgactgtgacctgaatcgcatttttgaccttgaaaatcttggcaaaaaaatgtcagaagatttgccatatgaagtgagaagggctcaagaaataaatcatttatttggtccaaaagacagtgaagattcctatgacattatttttgaccttcacaacaccacctctaacatggggtgcactcttattcttgaggattccgggaataactttttaattcagatgtttcattacattaagacttctctggctccactaccctgctacgtttatctgattgagcatccttccctcaaatatgcgaccactcgttccatagccaagtatcctgtgggtatagaagttggtcctcagcctcaaggggttctgagagctgatatcttggatcaaatgagaaaaatgattaaacatgctcttgattttatacatcatttcaatgaaggaaaagaatttcctccctgcgccattgaggtctataaaattatagagaaagttgattacccccgggatgaaaatggagaaattgctgctatcatccatcccaatctgcaggatcaagactggaaaccactgcatcctggggatcccatgtttttaactcttgatgggaagacgatcccactgggcggagactgtaccgtgtaccccgtgtttgtgaatgaggccgcatattacgaaaagaaagaagcttttgcaaagacaactaaactaacgctcaatgcaaaaagtattcgctgctgtttacattag

SEQ ID NO: 3 - protein, 313 aa, synthetic construct
  REGION 1..313: note = hASPA-R132G
MTSCHIAEEHIQKVAIFGGTHGNELTGVFLVKHWLENGAEIQRTGLEVKPFITNPRAVKKCTRYIDCDLNRIFDLENLGKKMSEDLPYEVRRAQEINHLFGPKDSEDSYDIIFDLHNTTSNMGCTLILEDSGNNFLIQMFHYIKTSLAPLPCYVYLIEHPSLKYATTRSIAKYPVGIEVGPQPQGVLRADILDQMRKMIKHALDFIHHFNEGKEFPPCAIEVYKIIEKVDYPRDENGEIAAIIHPNLQDQDWKPLHPGDPMFLTLDGKTIPLGGDCTVYPVFVNEAAYYEKKEAFAKTTKLTLNAKSIRCCLH

SEQ ID NO: 4 - DNA, 16 bp, synthetic construct
  misc_feature 1..16: note = TALEN-R
  source 1..16: mol_type = other DNA
tttctgtcaccaatcc

SEQ ID NO: 5 - DNA, 16 bp, synthetic construct
  misc_feature 1..16: note = TALEN-L
  source 1..16: mol_type = other DNA
cccctccaccccacag

SEQ ID NO: 6 - DNA, 18 bp, synthetic construct
  misc_feature 1..18: note = chr19
  source 1..18: mol_type = other DNA
ttttctgtcaccaatcct

SEQ ID NO: 7 - DNA, 18 bp, synthetic construct
  misc_feature 1..18: note = chr19
  source 1..18: mol_type = other DNA
ttatctgtcccctccacc

SEQ ID NO: 8 - DNA, 18 bp, synthetic construct
  misc_feature 1..18: note = chr19
  source 1..18: mol_type = other DNA
ttttatctgtcccctcca

SEQ ID NO: 9 - DNA, 18 bp, synthetic construct
  misc_feature 1..18: note = chr3
  source 1..18: mol_type = other DNA
atttctgtcaaaaatcct

SEQ ID NO: 10 - DNA, 18 bp, synthetic construct
  misc_feature 1..18: note = chr3
  source 1..18: mol_type = other DNA
ttgtctttcactaatact

SEQ ID NO: 11 - DNA, 18 bp, synthetic construct
  misc_feature 1..18: note = chr22
  source 1..18: mol_type = other DNA
ctccccccaccccccaaa

SEQ ID NO: 12 - DNA, 18 bp, synthetic construct
  misc_feature 1..18: note = chr22
  source 1..18: mol_type = other DNA
ttttctgtccccactcca

SEQ ID NO: 13 - DNA, 18 bp, synthetic construct
  misc_feature 1..18: note = chr5
  source 1..18: mol_type = other DNA
tactccccaccccacaga

SEQ ID NO: 14 - DNA, 18 bp, synthetic construct
  misc_feature 1..18: note = chr5
  source 1..18: mol_type = other DNA
tcccctgcatccaacagt

SEQ ID NO: 15 - DNA, 18 bp, synthetic construct
  misc_feature 1..18: note = chr17
  source 1..18: mol_type = other DNA
tccaccctacctcccagc

SEQ ID NO: 16 - DNA, 18 bp, synthetic construct
  misc_feature 1..18: note = chr17
  source 1..18: mol_type = other DNA
taccctcctccccacagt

SEQ ID NO: 17 - DNA, 18 bp, synthetic construct
  misc_feature 1..18: note = chr3
  source 1..18: mol_type = other DNA
ttttttgccaacactcct

SEQ ID NO: 18 - DNA, 18 bp, synthetic construct
  misc_feature 1..18: note = chr3
  source 1..18: mol_type = other DNA
ccaccaccaccccaccct

SEQ ID NO: 19 - DNA, 18 bp, synthetic construct
  misc_feature 1..18: note = chr20
  source 1..18: mol_type = other DNA
tcccctccaccccagttc

SEQ ID NO: 20 - DNA, 18 bp, synthetic construct
  misc_feature 1..18: note = chr20
  source 1..18: mol_type = other DNA
tcccctcaacccaaaact

SEQ ID NO: 21 - DNA, 18 bp, synthetic construct
  misc_feature 1..18: note = chr9
  source 1..18: mol_type = other DNA
tcccctcctgcccacaga

SEQ ID NO: 22 - DNA, 18 bp, synthetic construct
  misc_feature 1..18: note = chr9
  source 1..18: mol_type = other DNA
tcccctaccccccaaaca

SEQ ID NO: 23 - DNA, 18 bp, synthetic construct
  misc_feature 1..18: note = chr17
  source 1..18: mol_type = other DNA
ttctcaccaccccacact

SEQ ID NO: 24 - DNA, 18 bp, synthetic construct
  misc_feature 1..18: note = chr17
  source 1..18: mol_type = other DNA
tcccttcccacccaaagt

SEQ ID NO: 25 - DNA, 18 bp, synthetic construct
  misc_feature 1..18: note = chr5
  source 1..18: mol_type = other DNA
tcccctccacctcaaaca

SEQ ID NO: 26 - DNA, 18 bp, synthetic construct
  misc_feature 1..18: note = chr5
  source 1..18: mol_type = other DNA
cccccgccaccccatatt

SEQ ID NO: 27 - DNA, 18 bp, synthetic construct
  misc_feature 1..18: note = chr20
  source 1..18: mol_type = other DNA
tcacccccacccctcaat

SEQ ID NO: 28 - DNA, 18 bp, synthetic construct
  misc_feature 1..18: note = chr20
  source 1..18: mol_type = other DNA
ctctctatccacaatccc

SEQ ID NO: 29 - DNA, 18 bp, synthetic construct
  misc_feature 1..18: note = chr19
  source 1..18: mol_type = other DNA
tccactccacccccccaa

SEQ ID NO: 30 - DNA, 18 bp, synthetic construct
  misc_feature 1..18: note = chr19
  source 1..18: mol_type = other DNA
tcctctccaccaccccct

SEQ ID NO: 31 - DNA, 18 bp, synthetic construct
  misc_feature 1..18: note = chr22
  source 1..18: mol_type = other DNA
tccccccaccccccaaaa

SEQ ID NO: 32 - DNA, 18 bp, synthetic construct
  misc_feature 1..18: note = chr22
  source 1..18: mol_type = other DNA
ttttctgtccccactcca

SEQ ID NO: 33 - DNA, 18 bp, synthetic construct
  misc_feature 1..18: note = chr22
  source 1..18: mol_type = other DNA
ccccccaccccccaaaaa

SEQ ID NO: 34 - DNA, 18 bp, synthetic construct
  misc_feature 1..18: note = chr22
  source 1..18: mol_type = other DNA
ttttctgtccccactcca

SEQ ID NO: 35 - DNA, 18 bp, synthetic construct
  misc_feature 1..18: note = chrX
  source 1..18: mol_type = other DNA
tcttccatcactaattct

SEQ ID NO: 36 - DNA, 18 bp, synthetic construct
  misc_feature 1..18: note = chrX
  source 1..18: mol_type = other DNA
taccctccaccctaccat

SEQ ID NO: 37 - DNA, 18 bp, synthetic construct
  misc_feature 1..18: note = chr8
  source 1..18: mol_type = other DNA
ttttctgtaaccactcct

SEQ ID NO: 38 - DNA, 18 bp, synthetic construct
  misc_feature 1..18: note = chr8
  source 1..18: mol_type = other DNA
tgttctgacacttttccc

SEQ ID NO: 39 - DNA, 18 bp, synthetic construct
  misc_feature 1..18: note = chr10
  source 1..18: mol_type = other DNA
ccccctcccacccacctt

SEQ ID NO: 40 - DNA, 18 bp, synthetic construct
  misc_feature 1..18: note = chr10
  source 1..18: mol_type = other DNA
tcctccccaccccccagg

SEQ ID NO: 41 - DNA, 18 bp, synthetic construct
  misc_feature 1..18: note = chr10
  source 1..18: mol_type = other DNA
tcccctccccctcccacc

SEQ ID NO: 42 - DNA, 18 bp, synthetic construct
  misc_feature 1..18: note = chr10
  source 1..18: mol_type = other DNA
tcctccccaccccccagg

SEQ ID NO: 43 - DNA, 18 bp, synthetic construct
  misc_feature 1..18: note = chr19
  source 1..18: mol_type = other DNA
tcctcgccaccccccacg

SEQ ID NO: 44 - DNA, 18 bp, synthetic construct
  misc_feature 1..18: note = chr19
  source 1..18: mol_type = other DNA
cctcctccaccccactgt

SEQ ID NO: 45 - DNA, 18 bp, synthetic construct
  misc_feature 1..18: note = chr1
  source 1..18: mol_type = other DNA
ccacctccaccacacaca

SEQ ID NO: 46 - DNA, 18 bp, synthetic construct
  misc_feature 1..18: note = chr1
  source 1..18: mol_type = other DNA
tctcatccaacccacagg

SEQ ID NO: 47 - DNA, 18 bp, synthetic construct
  misc_feature 1..18: note = chr18
  source 1..18: mol_type = other DNA
ttttatatcaccaacccc

SEQ ID NO: 48 - DNA, 18 bp, synthetic construct
  misc_feature 1..18: note = chr18
  source 1..18: mol_type = other DNA
ttttctgtgatcaattat

SEQ ID NO: 49 - DNA, 18 bp, synthetic construct
  misc_feature 1..18: note = chr12
  source 1..18: mol_type = other DNA
ttctctctccccactcct

SEQ ID NO: 50 - DNA, 18 bp, synthetic construct
  misc_feature 1..18: note = chr12
  source 1..18: mol_type = other DNA
ttctctattaaaactcct

SEQ ID NO: 51 - DNA, 18 bp, synthetic construct
  misc_feature 1..18: note = chr6
  source 1..18: mol_type = other DNA
ccgtcagtcacccctcct

SEQ ID NO: 52 - DNA, 18 bp, synthetic construct
  misc_feature 1..18: note = chr6
  source 1..18: mol_type = other DNA
cccactccaccccactgt

SEQ ID NO: 53 - DNA, 18 bp, synthetic construct
  misc_feature 1..18: note = chr22
  source 1..18: mol_type = other DNA
cccccaccccccaaaaaa

SEQ ID NO: 54 - DNA, 18 bp, synthetic construct
  misc_feature 1..18: note = chr22
  source 1..18: mol_type = other DNA
ttttctgtccccactcca

SEQ ID NO: 55 - DNA, 18 bp, synthetic construct
  misc_feature 1..18: note = chr3
  source 1..18: mol_type = other DNA
tgccccccaacccaccaa

SEQ ID NO: 56 - DNA, 18 bp, synthetic construct
  misc_feature 1..18: note = chr3
  source 1..18: mol_type = other DNA
ttttctttcaccaatacc

SEQ ID NO: 57 - DNA, 18 bp, synthetic construct
  misc_feature 1..18: note = chr20
  source 1..18: mol_type = other DNA
acacctgcaccccacatt

SEQ ID NO: 58 - DNA, 18 bp, synthetic construct
  misc_feature 1..18: note = chr20
  source 1..18: mol_type = other DNA
ttttctgtctcaaaccat

SEQ ID NO: 59 - DNA, 18 bp, synthetic construct
  misc_feature 1..18: note = chr6
  source 1..18: mol_type = other DNA
tcccctcatccccacatc

SEQ ID NO: 60 - DNA, 18 bp, synthetic construct
  misc_feature 1..18: note = chr6
  source 1..18: mol_type = other DNA
tccccaacatcacacact

SEQ ID NO: 61 - DNA, 18 bp, synthetic construct
  misc_feature 1..18: note = chr16
  source 1..18: mol_type = other DNA
ttctgtggaaccaatact

SEQ ID NO: 62 - DNA, 18 bp, synthetic construct
  misc_feature 1..18: note = chr16
  source 1..18: mol_type = other DNA
tttgccgtcaccaaccct

SEQ ID NO: 63 - DNA, 18 bp, synthetic construct
  misc_feature 1..18: note = chr11
  source 1..18: mol_type = other DNA
ccccctccccccaaccct

SEQ ID NO: 64 - DNA, 18 bp, synthetic construct
  misc_feature 1..18: note = chr11
  source 1..18: mol_type = other DNA
tttgctgtccccacccca

SEQ ID NO: 65 - DNA, 18 bp, synthetic construct
  misc_feature 1..18: note = chr1
  source 1..18: mol_type = other DNA
cccactcccccccacccc

SEQ ID NO: 66 - DNA, 18 bp, synthetic construct
  misc_feature 1..18: note = chr1
  source 1..18: mol_type = other DNA
tccccactcccccacagc

SEQ ID NO: 67 - DNA, 18 bp, synthetic construct
  misc_feature 1..18: note = chr1
  source 1..18: mol_type = other DNA
ttttctattaacaaaaat

SEQ ID NO: 68 - DNA, 18 bp, synthetic construct
  misc_feature 1..18: note = chr1
  source 1..18: mol_type = other DNA
tcccctcctcaccactgt

SEQ ID NO: 69 - DNA, 18 bp, synthetic construct
  misc_feature 1..18: note = chr20
  source 1..18: mol_type = other DNA
tttcatctccccagccct

SEQ ID NO: 70 - DNA, 18 bp, synthetic construct
  misc_feature 1..18: note = chr20
  source 1..18: mol_type = other DNA
cctcctcctccccacagt

SEQ ID NO: 71 - DNA, 18 bp, synthetic construct
  misc_feature 1..18: note = chr8
  source 1..18: mol_type = other DNA
tttcctctccctaatcct

SEQ ID NO: 72 - DNA, 18 bp, synthetic construct
  misc_feature 1..18: note = chr8
  source 1..18: mol_type = other DNA
tcctctcctcccctccct

SEQ ID NO: 73 - DNA, 18 bp, synthetic construct
  misc_feature 1..18: note = chr8
  source 1..18: mol_type = other DNA
tttcctctccctaatcct

SEQ ID NO: 74 - DNA, 18 bp, synthetic construct
  misc_feature 1..18: note = chr8
  source 1..18: mol_type = other DNA
tcctctcctcccctccct

SEQ ID NO: 75 - DNA, 18 bp, synthetic construct
  misc_feature 1..18: note = chr1
  source 1..18: mol_type = other DNA
ttttctgaaaccaatcct

SEQ ID NO: 76 - DNA, 18 bp, synthetic construct
  misc_feature 1..18: note = chr1
  source 1..18: mol_type = other DNA
ctttttatccccagtaca

SEQ ID NO: 77 - DNA, 18 bp, synthetic construct
  misc_feature 1..18: note = chr6
  source 1..18: mol_type = other DNA
atttctttccccaaatct

SEQ ID NO: 78 - DNA, 18 bp, synthetic construct
  misc_feature 1..18: note = chr6
  source 1..18: mol_type = other DNA
ttttctgttacaaagcct

SEQ ID NO: 79 - DNA, 18 bp, synthetic construct
  misc_feature 1..18: note = chr7
  source 1..18: mol_type = other DNA
ttttcttacagcaacact

SEQ ID NO: 80 - DNA, 18 bp, synthetic construct
  misc_feature 1..18: note = chr7
  source 1..18: mol_type = other DNA
tttactgtcaccactact

SEQ ID NO: 81 - DNA, 18 bp, synthetic construct
  misc_feature 1..18: note = chr1
  source 1..18: mol_type = other DNA
tattctgtcatcactcct

SEQ ID NO: 82 - DNA, 18 bp, synthetic construct
  misc_feature 1..18: note = chr1
  source 1..18: mol_type = other DNA
tcccctcccacccttaac

SEQ ID NO: 83 - DNA, 18 bp, synthetic construct
  misc_feature 1..18: note = chr3
  source 1..18: mol_type = other DNA
tcccttccacctcaccaa

SEQ ID NO: 84 - DNA, 18 bp, synthetic construct
  misc_feature 1..18: note = chr3
  source 1..18: mol_type = other DNA
tgttctgtacccaaccct

SEQ ID NO: 85 - DNA, 18 bp, synthetic construct
  misc_feature 1..18: note = chr16
  source 1..18: mol_type = other DNA
ttctctccacaccacagt

SEQ ID NO: 86 - DNA, 18 bp, synthetic construct
  misc_feature 1..18: note = chr16
  source 1..18: mol_type = other DNA
attgcttacaccactcct

SEQ ID NO: 87 - DNA, 18 bp, synthetic construct
  misc_feature 1..18: note = chr3
  source 1..18: mol_type = other DNA
tccccaccaccccaaaga

SEQ ID NO: 88 - DNA, 18 bp, synthetic construct
  misc_feature 1..18: note = chr3
  source 1..18: mol_type = other DNA
ttttgaatcaacactccc

SEQ ID NO: 89 - DNA, 18 bp, synthetic construct
  misc_feature 1..18: note = chr16
  source 1..18: mol_type = other DNA
tcccctctcccccacagt

SEQ ID NO: 90 - DNA, 18 bp, synthetic construct
  misc_feature 1..18: note = chr16
  source 1..18: mol_type = other DNA
ccccctccacagcctcat

SEQ ID NO: 91 - DNA, 18 bp, synthetic construct
  misc_feature 1..18: note = chr1
  source 1..18: mol_type = other DNA
cccccccccccccacaat

SEQ ID NO: 92 - DNA, 18 bp, synthetic construct
  misc_feature 1..18: note = chr1
  source 1..18: mol_type = other DNA
ccccccccgccccccccc

SEQ ID NO: 93 - DNA, 18 bp, synthetic construct
  misc_feature 1..18: note = chr2
  source 1..18: mol_type = other DNA
tccccccaacccttcatt

SEQ ID NO: 94 - DNA, 18 bp, synthetic construct
  misc_feature 1..18: note = chr2
  source 1..18: mol_type = other DNA
cccctcccaccccccagt

SEQ ID NO: 95 - DNA, 18 bp, synthetic construct
  misc_feature 1..18: note = chr3
  source 1..18: mol_type = other DNA
ttctctgtcaccagccca

SEQ ID NO: 96 - DNA, 18 bp, synthetic construct
  misc_feature 1..18: note = chr3
  source 1..18: mol_type = other DNA
ttttaccttaacaatcct

SEQ ID NO: 97 - DNA, 18 bp, synthetic construct
  misc_feature 1..18: note = chr5
  source 1..18: mol_type = other DNA
ttttctataactcatatt

SEQ ID NO: 98 - DNA, 18 bp, synthetic construct
  misc_feature 1..18: note = chr5
  source 1..18: mol_type = other DNA
ccccctccaccccaaaaa

SEQ ID NO: 99 - DNA, 18 bp, synthetic construct
  misc_feature 1..18: note = chr4
  source 1..18: mol_type = other DNA
tttcttttcactattcct

SEQ ID NO: 100 - DNA, 18 bp, synthetic construct
  misc_feature 1..18: note = chr4
  source 1..18: mol_type = other DNA
ttttacatcaacaatcct

SEQ ID NO: 101 - DNA, 18 bp, synthetic construct
  misc_feature 1..18: note = chr13
  source 1..18: mol_type = other DNA
ttttctttctgcaaccat

SEQ ID NO: 102 - DNA, 18 bp, synthetic construct
  misc_feature 1..18: note = chr13
  source 1..18: mol_type = other DNA
tccacaccaccctacagt

SEQ ID NO: 103 - DNA, 18 bp, synthetic construct
  misc_feature 1..18: note = chr16
  source 1..18: mol_type = other DNA
cccccttcaccccacccc

SEQ ID NO: 104 - DNA, 18 bp, synthetic construct
  misc_feature 1..18: note = chr16
  source 1..18: mol_type = other DNA
ccccccccacaccactgc

SEQ ID NO: 105 - DNA, 18 bp, synthetic construct
  misc_feature 1..18: note = chr1
  source 1..18: mol_type = other DNA
tatgctgtctccatgcct

SEQ ID NO: 106 - DNA, 18 bp, synthetic construct
  misc_feature 1..18: note = chr1
  source 1..18: mol_type = other DNA
tcttctgccaccagtcct

SEQ ID NO: 107 - DNA, 18 bp, synthetic construct
  misc_feature 1..18: note = chr2
  source 1..18: mol_type = other DNA
acccctccaaccctcagc

SEQ ID NO: 108 - DNA, 18 bp, synthetic construct
  misc_feature 1..18: note = chr2
  source 1..18: mol_type = other DNA
ccccctcatcctcccagt

SEQ ID NO: 109 - DNA, 18 bp, synthetic construct
  misc_feature 1..18: note = chr5
  source 1..18: mol_type = other DNA
ccacctccacctaacagt

SEQ ID NO: 110 - DNA, 18 bp, synthetic construct
  misc_feature 1..18: note = chr5
  source 1..18: mol_type = other DNA
tcttctgacaccatccca

SEQ ID NO: 111 - DNA, 18 bp, synthetic construct
  misc_feature 1..18: note = chr15
  source 1..18: mol_type = other DNA
tccactccaccccctctt

SEQ ID NO: 112 - DNA, 18 bp, synthetic construct
  misc_feature 1..18: note = chr15
  source 1..18: mol_type = other DNA
ccccctcaaccacccagc

SEQ ID NO: 113 - DNA, 18 bp, synthetic construct
  misc_feature 1..18: note = chr2
  source 1..18: mol_type = other DNA
tcccatctcacccaaaat

SEQ ID NO: 114 - DNA, 18 bp, synthetic construct
  misc_feature 1..18: note = chr2
  source 1..18: mol_type = other DNA
ttttccatcaccaattca

SEQ ID NO: 115 - DNA, 18 bp, synthetic construct
  misc_feature 1..18: note = chr8
  source 1..18: mol_type = other DNA
ttgtcttccacaaaacct

SEQ ID NO: 116 - DNA, 18 bp, synthetic construct
  misc_feature 1..18: note = chr8
  source 1..18: mol_type = other DNA
ctttctatcagcactcct

SEQ ID NO: 117 - DNA, 18 bp, synthetic construct
  misc_feature 1..18: note = chr2
  source 1..18: mol_type = other DNA
tcccctcccccacaatct

SEQ ID NO: 118 - DNA, 18 bp, synthetic construct
  misc_feature 1..18: note = chr2
  source 1..18: mol_type = other DNA
tccccccacccccaccac

SEQ ID NO: 119 - DNA, 18 bp, synthetic construct
  misc_feature 1..18: note = chr8
  source 1..18: mol_type = other DNA
ttctctgtgcacaagcct

SEQ ID NO: 120 - DNA, 18 bp, synthetic construct
  misc_feature 1..18: note = chr8
  source 1..18: mol_type = other DNA
tcttctgtcacctgtcct

SEQ ID NO: 121 - DNA, 18 bp, synthetic construct
  misc_feature 1..18: note = chr8
  source 1..18: mol_type = other DNA
tccccaccctcccacatt

SEQ ID NO: 122 - DNA, 18 bp, synthetic construct
  misc_feature 1..18: note = chr8
  source 1..18: mol_type = other DNA
ctctctggcacccttcct

SEQ ID NO: 123 - DNA, 18 bp, synthetic construct
  misc_feature 1..18: note = chr9
  source 1..18: mol_type = other DNA
tatccagacacccaccct

SEQ ID NO: 124 - DNA, 18 bp, synthetic construct
  misc_feature 1..18: note = chr9
  source 1..18: mol_type = other DNA
ccccatccaccccacaca

SEQ ID NO: 125 - DNA, 18 bp, synthetic construct
  misc_feature 1..18: note = chr11
  source 1..18: mol_type = other DNA
acctctgcctcccatcct

SEQ ID NO: 126 - DNA, 18 bp, synthetic construct
  misc_feature 1..18: note = chr11
  source 1..18: mol_type = other DNA
ccccctccaccccccgat

SEQ ID NO: 127 - DNA, 18 bp, synthetic construct
  misc_feature 1..18: note = chr1
  source 1..18: mol_type = other DNA
ccacccccacccccccac

SEQ ID NO: 128 - DNA, 18 bp, synthetic construct
  misc_feature 1..18: note = chr1
  source 1..18: mol_type = other DNA
acccctcccccgcacagt

SEQ ID NO: 129 - DNA, 18 bp, synthetic construct
  misc_feature 1..18: note = chr5
  source 1..18: mol_type = other DNA
atttctttcccccatact

SEQ ID NO: 130 - DNA, 18 bp, synthetic construct
  misc_feature 1..18: note = chr5
  source 1..18: mol_type = other DNA
ttccctgccaccaacccc

SEQ ID NO: 131 - DNA, 18 bp, synthetic construct
  misc_feature 1..18: note = chr5
  source 1..18: mol_type = other DNA
ctttctctcacctattct

SEQ ID NO: 132 - DNA, 18 bp, synthetic construct
  misc_feature 1..18: note = chr5
  source 1..18: mol_type = other DNA
tactcagtatccaatcct

SEQ ID NO: 133 - DNA, 18 bp, synthetic construct
  misc_feature 1..18: note = chr12
  source 1..18: mol_type = other DNA
tctcacccacccaacagt

SEQ ID NO: 134 - DNA, 18 bp, synthetic construct
  misc_feature 1..18: note = chr12
  source 1..18: mol_type = other DNA
tgccctcctacccacaac

SEQ ID NO: 135 - DNA, 18 bp, synthetic construct
  misc_feature 1..18: note = chr7
  source 1..18: mol_type = other DNA
tcttctgtcactaaaact

SEQ ID NO: 136 - DNA, 18 bp, synthetic construct
  misc_feature 1..18: note = chr7
  source 1..18: mol_type = other DNA
ctttctggcacatatact

SEQ ID NO: 137 - DNA, 18 bp, synthetic construct
  misc_feature 1..18: note = chr3
  source 1..18: mol_type = other DNA
gccccccaacccaccaaa

SEQ ID NO: 138 - DNA, 18 bp, synthetic construct
  misc_feature 1..18: note = chr3
  source 1..18: mol_type = other DNA
ttttctttcaccaatacc

SEQ ID NO: 139 - DNA, 18 bp, synthetic construct
  misc_feature 1..18: note = chr11
  source 1..18: mol_type = other DNA
tcccccccaccccgccaa

SEQ ID NO: 140 - DNA, 18 bp, synthetic construct
  misc_feature 1..18: note = chr11
  source 1..18: mol_type = other DNA
ccacctccaccacaaggt

SEQ ID NO: 141 - DNA, 18 bp, synthetic construct
  misc_feature 1..18: note = chr5
  source 1..18: mol_type = other DNA
tttcccttcaaacatcca

SEQ ID NO: 142 - DNA, 18 bp, synthetic construct
  misc_feature 1..18: note = chr5
  source 1..18: mol_type = other DNA
ctttctgtcacccatcat

SEQ ID NO: 143 - DNA, 18 bp, synthetic construct
  misc_feature 1..18: note = chr19
  source 1..18: mol_type = other DNA
cccctccaaccccatagt

SEQ ID NO: 144 - DNA, 18 bp, synthetic construct
  misc_feature 1..18: note = chr19
  source 1..18: mol_type = other DNA
tctcctgtcaccaaatct

SEQ ID NO: 145 - DNA, 18 bp, synthetic construct
  misc_feature 1..18: note = chr9
  source 1..18: mol_type = other DNA
tcccccccaccccaccca

SEQ ID NO: 146 - DNA, 18 bp, synthetic construct
  misc_feature 1..18: note = chr9
  source 1..18: mol_type = other DNA
tttcttctcccctaacct

SEQ ID NO: 147 - DNA, 18 bp, synthetic construct
  misc_feature 1..18: note = chr21
  source 1..18: mol_type = other DNA
tcctttcccaccaatcct

SEQ ID NO: 148 - DNA, 18 bp, synthetic construct
  misc_feature 1..18: note = chr21
  source 1..18: mol_type = other DNA
ttccttccaccccactgc

SEQ ID NO: 149 - DNA, 18 bp, synthetic construct
  misc_feature 1..18: note = chr6
  source 1..18: mol_type = other DNA
ttttctgtccccaaacag

SEQ ID NO: 150 - DNA, 18 bp, synthetic construct
  misc_feature 1..18: note = chr6
  source 1..18: mol_type = other DNA
tcctccccaccaccaaat

SEQ ID NO: 151 - DNA, 18 bp, synthetic construct
  misc_feature 1..18: note = chr14
  source 1..18: mol_type = other DNA
tccccccccccccccaca

SEQ ID NO: 152 - DNA, 18 bp, synthetic construct
  misc_feature 1..18: note = chr14
  source 1..18: mol_type = other DNA
tcacccccaacacacaca

SEQ ID NO: 153 - DNA, 18 bp, synthetic construct
  misc_feature 1..18: note = chr12
  source 1..18: mol_type = other DNA
ccccctccccagcacagg

SEQ ID NO: 154 - DNA, 18 bp, synthetic construct
  misc_feature 1..18: note = chr12
  source 1..18: mol_type = other DNA
ttccctccactccactgt

SEQ ID NO: 155 - DNA, 18 bp, synthetic construct
  misc_feature 1..18: note = chr16
  source 1..18: mol_type = other DNA
ctccctccaccccactca

SEQ ID NO: 156 - DNA, 18 bp, synthetic construct
  misc_feature 1..18: note = chr16
  source 1..18: mol_type = other DNA
tcccctccttcccccaga

SEQ ID NO: 157 - DNA, 18 bp, synthetic construct
  misc_feature 1..18: note = chr1
  source 1..18: mol_type = other DNA
ccccctccactccaaaag

SEQ ID NO: 158 - DNA, 18 bp, synthetic construct
  misc_feature 1..18: note = chr1
  source 1..18: mol_type = other DNA
ttttctcttcccactccc

SEQ ID NO: 159 - DNA, 18 bp, synthetic construct
  misc_feature 1..18: note = chrX
  source 1..18: mol_type = other DNA
taccccctcccaaacagt

SEQ ID NO: 160 - DNA, 18 bp, synthetic construct
  misc_feature 1..18: note = chrX
  source 1..18: mol_type = other DNA
gcccctcccccccccact

SEQ ID NO: 161 - DNA, 18 bp, synthetic construct
  misc_feature 1..18: note = chr12
  source 1..18: mol_type = other DNA
tctgctgccaccagtcac

SEQ ID NO: 162 - DNA, 18 bp, synthetic construct
  misc_feature 1..18: note = chr12
  source 1..18: mol_type = other DNA
tcccttccacccctcact

SEQ ID NO: 163 - DNA, 18 bp, synthetic construct
  misc_feature 1..18: note = chr14
  source 1..18: mol_type = other DNA
tcccctccaacccaaacc

SEQ ID NO: 164 - DNA, 18 bp, synthetic construct
  misc_feature 1..18: note = chr14
  source 1..18: mol_type = other DNA
ctttcattcacaatccct

SEQ ID NO: 165 - DNA, 18 bp, synthetic construct
  misc_feature 1..18: note = chr7
  source 1..18: mol_type = other DNA
tcttatttcactaagact

SEQ ID NO: 166 - DNA, 18 bp, synthetic construct
  misc_feature 1..18: note = chr7
  source 1..18: mol_type = other DNA
tttcctctcaccagtcct

SEQ ID NO: 167 - DNA, 18 bp, synthetic construct
  misc_feature 1..18: note = chr18
  source 1..18: mol_type = other DNA
agccctccaacccacagt

SEQ ID NO: 168 - DNA, 18 bp, synthetic construct
  misc_feature 1..18: note = chr18
  source 1..18: mol_type = other DNA
tcttttcctccccacaat

SEQ ID NO: 169 - DNA, 18 bp, synthetic construct
  misc_feature 1..18: note = chr5
  source 1..18: mol_type = other DNA
tccctcccaccacacagc

SEQ ID NO: 170 - DNA, 18 bp, synthetic construct
  misc_feature 1..18: note = chr5
  source 1..18: mol_type = other DNA
acttccccaccccacagc

SEQ ID NO: 171 - DNA, 18 bp, synthetic construct
  misc_feature 1..18: note = chr13
  source 1..18: mol_type = other DNA
tcacctccccccaaaaat

SEQ ID NO: 172 - DNA, 18 bp, synthetic construct
  misc_feature 1..18: note = chr13
  source 1..18: mol_type = other DNA
tcttcactccccaaaact

SEQ ID NO: 173 - DNA, 18 bp, synthetic construct
  misc_feature 1..18: note = chr7
  source 1..18: mol_type = other DNA
ttttctgtcttccatcaa

SEQ ID NO: 174 - DNA, 18 bp, synthetic construct
  misc_feature 1..18: note = chr7
  source 1..18: mol_type = other DNA
ttttctttagcccatcct

SEQ ID NO: 175 - DNA, 18 bp, synthetic construct
  misc_feature 1..18: note = chr9
  source 1..18: mol_type = other DNA
tttcctgtcaacactcct

SEQ ID NO: 176 - DNA, 18 bp, synthetic construct
  misc_feature 1..18: note = chr9
  source 1..18: mol_type = other DNA
cattccttcaaaaatcat

SEQ ID NO: 177 - DNA, 18 bp, synthetic construct
  misc_feature 1..18: note = chr18
  source 1..18: mol_type = other DNA
ttttctctcctctctcct

SEQ ID NO: 178 - DNA, 18 bp, synthetic construct
  misc_feature 1..18: note = chr18
  source 1..18: mol_type = other DNA
ttttctggcatcattcat

SEQ ID NO: 179 - DNA, 18 bp, synthetic construct
  misc_feature 1..18: note = chr1
  source 1..18: mol_type = other DNA
tccccccaaacacacaca

SEQ ID NO: 180 - DNA, 18 bp, synthetic construct
  misc_feature 1..18: note = chr1
  source 1..18: mol_type = other DNA
cccccaccaccccccacc

SEQ ID NO: 181 - DNA, 18 bp, synthetic construct
  misc_feature 1..18: note = chr12
  source 1..18: mol_type = other DNA
ttccctctctctactcct

SEQ ID NO: 182 - DNA, 18 bp, synthetic construct
  misc_feature 1..18: note = chr12
  source 1..18: mol_type = other DNA
ctttctctccccaattct

SEQ ID NO: 183 - DNA, 18 bp, synthetic construct
  misc_feature 1..18: note = chr16
  source 1..18: mol_type = other DNA
ctttatctccccaagcaa

SEQ ID NO: 184 - DNA, 18 bp, synthetic construct
  misc_feature 1..18: note = chr16
  source 1..18: mol_type = other DNA
ttttctatcactaaacct

SEQ ID NO: 185 - DNA, 18 bp, synthetic construct
  misc_feature 1..18: note = chr4
  source 1..18: mol_type = other DNA
gtttatgtccccaatccc

SEQ ID NO: 186 - DNA, 18 bp, synthetic construct
  misc_feature 1..18: note = chr4
  source 1..18: mol_type = other DNA
atttatatccacaatcat

SEQ ID NO: 187 - DNA, 18 bp, synthetic construct
  misc_feature 1..18: note = chr19
  source 1..18: mol_type = other DNA
ccccctcctcgccacccc

SEQ ID NO: 188 - DNA, 18 bp, synthetic construct
  misc_feature 1..18: note = chr19
  source 1..18: mol_type = other DNA
cctcctccaccccactgt

SEQ ID NO: 189 - DNA, 18 bp, synthetic construct
  misc_feature 1..18: note = chr15
  source 1..18: mol_type = other DNA
tgttctgtaaccaatact

SEQ ID NO: 190 - DNA, 18 bp, synthetic construct
  misc_feature 1..18: note = chr15
  source 1..18: mol_type = other DNA
ttttcttgcccagaccct

SEQ ID NO: 191 - DNA, 18 bp, synthetic construct
  misc_feature 1..18: note = chr3
  source 1..18: mol_type = other DNA
caccccccaccccactcc

SEQ ID NO: 192 - DNA, 18 bp, synthetic construct
  misc_feature 1..18: note = chr3
  source 1..18: mol_type = other DNA
tcccacccacgccacact

SEQ ID NO: 193 - DNA, 18 bp, synthetic construct
  misc_feature 1..18: note = chr21
  source 1..18: mol_type = other DNA
tccccccccacccccagt

SEQ ID NO: 194 - DNA, 18 bp, synthetic construct
  misc_feature 1..18: note = chr21
  source 1..18: mol_type = other DNA
tattcattcaacaaacat

SEQ ID NO: 195 - DNA, 18 bp, synthetic construct
  misc_feature 1..18: note = chr10
  source 1..18: mol_type = other DNA
ttttcccttaccaatcta

SEQ ID NO: 196 - DNA, 18 bp, synthetic construct
  misc_feature 1..18: note = chr10
  source 1..18: mol_type = other DNA
ttttccctcaccaatcta

SEQ ID NO: 197 - DNA, 18 bp, synthetic construct
  misc_feature 1..18: note = chr20
  source 1..18: mol_type = other DNA
cccccacccccccacaca

SEQ ID NO: 198 - DNA, 18 bp, synthetic construct
  misc_feature 1..18: note = chr20
  source 1..18: mol_type = other DNA
tcctctcccaccaatcac

SEQ ID NO: 199 - DNA, 18 bp, synthetic construct
  misc_feature 1..18: note = chr1
  source 1..18: mol_type = other DNA
cccccccccccccacaat

SEQ ID NO: 200 - DNA, 18 bp, synthetic construct
  misc_feature 1..18: note = chr1
  source 1..18: mol_type = other DNA
tccccccccccgcccccc

SEQ ID NO: 201 - DNA, 18 bp, synthetic construct
  misc_feature 1..18: note = chr4
  source 1..18: mol_type = other DNA
ttccctccttcccccaac

SEQ ID NO: 202 - DNA, 18 bp, synthetic construct
  misc_feature 1..18: note = chr4
  source 1..18: mol_type = other DNA
tttcctgtcaccatttct

SEQ ID NO: 203 - DNA, 21 bp, synthetic construct
  misc_feature 1..21: note = ASPA-Exon1 forward primer
  source 1..21: mol_type = other DNA
ctccactcaagggaattctgt

SEQ ID NO: 204 - DNA, 20 bp, synthetic construct
  misc_feature 1..20: note = ASPA-Exon1 reverse primer
  source 1..20: mol_type = other DNA
actgcatgtacggacatgaa

SEQ ID NO: 205 - DNA, 19 bp, synthetic construct
  misc_feature 1..19: note = ASPA-Exon2 forward primer
  source 1..19: mol_type = other DNA
agatttggcgactggttct

SEQ ID NO: 206 - DNA, 20 bp, synthetic construct
  misc_feature 1..20: note = ASPA-Exon2 reverse primer
  source 1..20: mol_type = other DNA
tgcaccttccctcataactg

SEQ ID NO: 207 - DNA, 20 bp, synthetic construct
  misc_feature 1..20: note = ASPA-Exon3 forward primer
  source 1..20: mol_type = other DNA
actctgttgaagcaaagaga

SEQ ID NO: 208 - DNA, 20 bp, synthetic construct
  misc_feature 1..20: note = ASPA-Exon3 reverse primer
  source 1..20: mol_type = other DNA
cagagcaagactctgtctca

SEQ ID NO: 209 - DNA, 20 bp, synthetic construct
  misc_feature 1..20: note = ASPA-Exon4 forward primer
  source 1..20: mol_type = other DNA
ttccatgatgctacatggtt

SEQ ID NO: 210 - DNA, 21 bp, synthetic construct
  misc_feature 1..21: note = ASPA-Exon4 reverse primer
  source 1..21: mol_type = other DNA
gcaaatctgacccaggttcca

SEQ ID NO: 211 - DNA, 20 bp, synthetic construct
  misc_feature 1..20: note = ASPA-Exon5 forward primer
  source 1..20: mol_type = other DNA
tgttctcgaactcctgacct

SEQ ID NO: 212 - DNA, 20 bp, synthetic construct
  misc_feature 1..20: note = ASPA-Exon5 reverse primer
  source 1..20: mol_type = other DNA
gcgaagtgctgtatgagcta

SEQ ID NO: 213 - DNA, 19 bp, synthetic construct
  misc_feature 1..19: note = ASPA-Exon6 forward primer
  source 1..19: mol_type = other DNA
gatcaagactggaaaccac

SEQ ID NO: 214 - DNA, 20 bp, synthetic construct
  misc_feature 1..20: note = ASPA-Exon6 reverse primer
  source 1..20: mol_type = other DNA
gaagtgtagtaaggcaaagc

SEQ ID NO: 215 - DNA, 20 bp, synthetic construct
  misc_feature 1..20: note = Endo-OCT4 forward primer
  source 1..20: mol_type = other DNA
cctcacttcactgcactgta

SEQ ID NO: 216 - DNA, 20 bp, synthetic construct
  misc_feature 1..20: note = Endo-OCT4 reverse primer
  source 1..20: mol_type = other DNA
caggttttctttccctagct

SEQ ID NO: 217 - DNA, 19 bp, synthetic construct
  misc_feature 1..19: note = Endo-SOX2 forward primer
  source 1..19: mol_type = other DNA
cccagcagacttcacatgt

SEQ ID NO: 218 - DNA, 20 bp, synthetic construct
  misc_feature 1..20: note = Endo-SOX2 reverse primer
  source 1..20: mol_type = other DNA
cctcccatttccctcgtttt

SEQ ID NO: 219 - DNA, 21 bp, synthetic construct
  misc_feature 1..21: note = Endo-NANOG forward primer
  source 1..21: mol_type = other DNA
gaatcttcacctatgcctgtg

SEQ ID NO: 220 - DNA, 21 bp, synthetic construct
  misc_feature 1..21: note = Endo-NANOG reverse primer
  source 1..21: mol_type = other DNA
atcattgagtacacacagctg

SEQ ID NO: 221 - DNA, 21 bp, synthetic construct
  misc_feature 1..21: note = Exo-OCT4 forward primer
  source 1..21: mol_type = other DNA
ctctagagcctctgctaacca

SEQ ID NO: 222 - DNA, 21 bp, synthetic construct
  misc_feature 1..21: note = Exo-OCT4 reverse primer
  source 1..21: mol_type = other DNA
tgtgcatagtcgctgcttgat

SEQ ID NO: 223 - DNA, 21 bp, synthetic construct
  misc_feature 1..21: note = Exo-KLF4 forward primer
  source 1..21: mol_type = other DNA
gctcccatctttctccacgtt

SEQ ID NO: 224 - DNA, 22 bp, synthetic construct
  misc_feature 1..22: note = Exo-KLF4 reverse primer
  source 1..22: mol_type = other DNA
gaagcttgaattcctgcaggca

SEQ ID NO: 225 - DNA, 21 bp, synthetic construct
  misc_feature 1..21: note = Exo-LIN28 forward primer
  source 1..21: mol_type = other DNA
agagcatcagccatatggtag

SEQ ID NO: 226 - DNA, 22 bp, synthetic construct
  misc_feature 1..22: note = Exo-LIN28 reverse primer
  source 1..22: mol_type = other DNA
gaagcttgaattcctgcaggca

SEQ ID NO: 227 - DNA, 21 bp, synthetic construct
  misc_feature 1..21: note = Exo-L-MYC forward primer
  source 1..21: mol_type = other DNA
ctctagagcctctgctaacca

SEQ ID NO: 228 - DNA, 21 bp, synthetic construct
  misc_feature 1..21: note = Exo-L-MYC reverse primer
  source 1..21: mol_type = other DNA
tcgaatttcttccagatgtcc

SEQ ID NO: 229 - DNA, 23 bp, synthetic construct
  misc_feature 1..23: note = ASPA forward primer
  source 1..23: mol_type = other DNA
cggaattcatgacttcttgtcac

SEQ ID NO: 230 - DNA, 24 bp, synthetic construct
  misc_feature 1..24: note = ASPA reverse primer
  source 1..24: mol_type = other DNA
ggactagtctaatgtaaacagcag

SEQ ID NO: 231 - DNA, 22 bp, synthetic construct
  misc_feature 1..22: note = ACTIN forward primer
  source 1..22: mol_type = other DNA
ccgcaaagacctgtacgccaac

SEQ ID NO: 232 - DNA, 22 bp, synthetic construct
  misc_feature 1..22: note = ACTIN reverse primer
  source 1..22: mol_type = other DNA
ccagggcagtgatctccttctg

SEQ ID NO: 233 - DNA, 20 bp, synthetic construct
  misc_feature 1..20: note = SOX1 forward primer
  source 1..20: mol_type = other DNA
aatactggagacgaacgccg

SEQ ID NO: 234 - DNA, 20 bp, synthetic construct
  misc_feature 1..20: note = SOX1 reverse primer
  source 1..20: mol_type = other DNA
agtgcttggacctgccttac

SEQ ID NO: 235 - DNA, 20 bp, synthetic construct
  misc_feature 1..20: note = PAX6 forward primer
  source 1..20: mol_type = other DNA
gtgtccaacggatgtgtgag

SEQ ID NO: 236 - DNA, 20 bp, synthetic construct
  misc_feature 1..20: note = PAX6 reverse primer
  source 1..20: mol_type = other DNA
ctagccaggttgcgaagaac

SEQ ID NO: 237 - DNA, 20 bp, synthetic construct
  misc_feature 1..20: note = AAVS1 forward primer
  source 1..20: mol_type = other DNA
ctctaacgctgccgtctctc

SEQ ID NO: 238 - DNA, 20 bp, synthetic construct
  misc_feature 1..20: note = AAVS1 reverse primer
  source 1..20: mol_type = other DNA
gcttctcctcttgggaagtg

SEQ ID NO: 239 - DNA, 20 bp, synthetic construct
  misc_feature 1..20: note = ASPA reverse primer
  source 1..20: mol_type = other DNA
agctcattcccatgggttcc

SEQ ID NO: 240 - DNA, 21 bp, synthetic construct
  misc_feature 1..21: note = PBS/psi forwad primer
  source 1..21: mol_type = other DNA
acttgaaagcgaaagggaaac

SEQ ID NO: 241 - DNA, 19 bp, synthetic construct
  misc_feature 1..19: note = PBS/psi reverse primer
  source 1..19: mol_type = other DNA
tttggcgtactcaccagtc

SEQ ID NO: 242 - DNA, 24 bp, synthetic construct
  misc_feature 1..24: note = PBS/psi-TaqMan probe
  source 1..24: mol_type = other DNA
agctctctcgacgcaggactcggc

SEQ ID NO: 243 - DNA, 25 bp, synthetic construct
  misc_feature 1..25: note = Albumin forward primer
  source 1..25: mol_type = other DNA
tgaaacatacgttcccaaagagttt

SEQ ID NO: 244 - DNA, 26 bp, synthetic construct
  misc_feature 1..26: note = Albumin reverse primer
  source 1..26: mol_type = other DNA
ctctccttctcagaaagtgtgcatat

SEQ ID NO: 245 - DNA, 27 bp, synthetic construct
  misc_feature 1..27: note = Albumin-TaqMan probe
  source 1..27: mol_type = other DNA
tgctgaaacattcaccttccatgcaga

SEQ ID NO: 246 - DNA, 150 bp, synthetic construct
  misc_feature 1..150: note = PBS/psi-gBlock
  source 1..150: mol_type = other DNA
tctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggct

SEQ ID NO: 247 - DNA, 125 bp, synthetic construct
  misc_feature 1..125: note = Albumin-gBlock
  source 1..125: mol_type = other DNA
catggcggccgcgggaattcgatttgaaacatacgttcccaaagagtttaatgctgaaacattcaccttccatgcagatatatgcacactttctgagaaggagagaatcactagtgaattcgcgg

SEQ ID NO: 248 - DNA, 12 bp, synthetic construct
  source 1..12: mol_type = other DNA
caatgcaaaaaa

SEQ ID NO: 249 - DNA, 12 bp, synthetic construct
  source 1..12: mol_type = other DNA
cctagatgatag

SEQ ID NO: 250 - DNA, 12 bp, synthetic construct
  source 1..12: mol_type = other DNA
caatgcaaaaaa

SEQ ID NO: 251 - DNA, 12 bp, synthetic construct
  source 1..12: mol_type = other DNA
cctagatgatag

SEQ ID NO: 252 - DNA, 11 bp, synthetic construct
  source 1..11: mol_type = other DNA
gaatgcggccg

SEQ ID NO: 253 - DNA, 15 bp, synthetic construct
  source 1..15: mol_type = other DNA
agcaaaaaaaatgtc

SEQ ID NO: 254 - DNA, 11 bp, synthetic construct
  source 1..11: mol_type = other DNA
catccgtccta

SEQ ID NO: 255 - DNA, 15 bp, synthetic construct
  source 1..15: mol_type = other DNA
ttattcttttggaag

SEQ ID NO: 256 - DNA, 12 bp, synthetic construct
  source 1..12: mol_type = other DNA
ccactctgttcc

SEQ ID NO: 257 - DNA, 13 bp, synthetic construct
  source 1..13: mol_type = other DNA
gattactccccgg

(19) United States
(12) Patent Application Publication
(10) Pub. No.: US 2023/0416689 A1
(43) Pub. Date: Dec. 28, 2023

(54) TREATMENT OF CANAVAN DISEASE

(71) Applicant: CITY OF HOPE, Duarte, CA (US)

(72) Inventors: Yanhong SHI, Duarte, CA (US); Lizhao FENG, Duarte, CA (US); Jianfei CHAO, Duarte, CA (US); Guihua SUN, Duarte, CA (US)

(73) Assignee: CITY OF HOPE, Duarte, CA (US)

(21) Appl. No.: 18/296,224

(22) Filed: Apr. 5, 2023

Related U.S. Application Data
(63) Continuation of application No. PCT/US2021/052467, filed on Sep. 28, 2021.
(60) Provisional application No. 63/087,569, filed on Oct. 5, 2020.

(51) Int. Cl. C12N 5/074; C12N 5/0793; C12N 15/86; C12N 15/11

(52) U.S. Cl. CPC C12N 5/0696; C12N 5/0619; C12N 2310/20; C12N 15/11; C12N 15/86

(57) ABSTRACT

Disclosed herein are methods of treating Canavan disease in a subject through restoring ASPA enzymatic activities in the subject by expressing an exogenous functional ASPA gene in the brain of the subject. Also disclosed are a process of producing neural precursor cells, including NPCs, glial progenitor cells and OPCs, which express an exogenous functional ASPA gene and the neural precursor cells produced by this process.

### PRIORITY CLAIM

This application is a continuation of International Patent Application No. PCT/US2021/052467, filed Sep. 28, 2021, which claims the benefit of U.S. Provisional Patent Application No. 63/087,569, filed Oct. 5, 2020, both of which are incorporated herein by reference in their entirety, including drawings.

### SEQUENCE LISTING

This application contains a ST.26 compliant Sequence Listing, which was submitted in XML format via Patent Center, and is hereby incorporated by reference in its entirety. The XML copy, created on Mar. 29, 2023, is named 0544358210US01.xml and is 314,000 bytes in size.

### BACKGROUND

Canavan disease (CD) is a rare, autosomal recessive neurodevelopmental disorder that affects children from infancy[1]. Most children with infantile-onset CD, the most prevalent form of the disease, will die within the first decade of life. There is neither a cure nor a standard treatment for this disease. CD is caused by genetic mutation in the aspartoacylase (ASPA) gene, which encodes a metabolic enzyme synthesized by oligodendrocytes in the brain [1]. The ASPA enzyme breaks down N-acetyl-aspartate (NAA), an amino acid derivative in the brain. The cycle of production and breakdown of NAA appears to be critical for maintaining the white matter of the brain, which consists of nerve fibers covered by myelin. Mutation of the ASPA gene results in a deficiency in the ASPA enzyme, which in turn leads to accumulation of the NAA substrate, spongy degeneration (vacuolation) and myelination defect in the brain. The clinical symptoms of CD include impaired motor function, mental retardation, and early death [2].

There is currently no approved therapy for this condition. The closest therapeutic candidate under clinical development for this disease is the delivery of a functional ASPA gene directly into the brain via adeno-associated viral (AAV) transduction [3] or liposome-mediated transfection [4]. The AAV product has undergone a phase 1 clinical trial with 13 patients, while the liposome ASPA gene transfer has been tested in 2 patients. The results of the studies showed reasonable safety profiles, however, the clinical benefits to the patients were limited [3-4]. There is a clear, unmet medical need for an effective therapy for CD. This disclosure satisfies this need.

### SUMMARY

In one aspect, this disclosure relates to a method of treating Canavan disease in a subject. The method entails restoring ASPA enzymatic activities in the subject by expressing an exogenous functional ASPA gene in the brain of the subject. In some embodiments, the ASPA enzymatic activities are restored by providing a functional ASPA-expressing neural precursor cells, including neural progenitor cells (NPCs), glial progenitor cells, and oligodendroglial progenitor cells (OPCs), to the brain of the subject.

In a related aspect, this disclosure relates to neural precursor cells, including NPCs, glial progenitor cells, and OPCs, which express an exogenous functional ASPA gene produced by a process comprising the steps of reprogramming or converting somatic cells isolated from a subject suffering from Canavan disease into induced pluripotent stem cells (iPSCs), introducing a functional ASPA gene in the reprogrammed or converted iPSCs to obtain genetically corrected iPSCs which express a functional ASPA, and differentiating the genetically corrected iPSCs into neural precursor cells, including NPCs, glial progenitor cells and OPCs. Alternatively, the neural precursor cells, including NPCs, glial progenitor cells and OPCs, which express an exogenous functional ASPA gene are produced by a process comprising the steps of reprogramming or converting somatic cells isolated from a subject suffering from Canavan disease into iPSCs, differentiating the reprogrammed iPSCs into neural precursor cells, and introducing a functional ASPA gene into the neural precursor cells to obtain genetically corrected neural precursor cells which express a functional ASPA.

In another aspect, this disclosure relates to a method of treating Canavan disease in a subject. The method entails the steps of reprogramming or converting somatic cells isolated from the subject suffering from Canavan disease into iPSCs, introducing a functional ASPA gene in the reprogrammed or converted iPSCs to obtain genetically corrected iPSCs which express a functional ASPA, differentiating the genetically corrected iPSCs into neural precursor cells, including NPCs, glial progenitor cells and OPCs, and transplanting the neural precursor cells into the brain of the subject. Alternatively, the method entails the steps of reprogramming or converting somatic cells isolated from the subject suffering from Canavan disease into iPSCs, differentiating the iPSCs into neural precursor cells, including NPCs, glial progenitor cells and OPCs, introducing a functional ASPA gene into the neural precursor cells to obtain genetically corrected neural precursor cells which express a functional ASPA, and transplanting the genetically corrected neural precursor cells into the brain of the subject.

In another aspect, this disclosure relates to a method of producing functional ASPA-expressing neural precursor cells which serve as a source of the ASPA enzyme for treating Canavan disease. The method includes the steps of reprogramming or converting somatic cells isolated from a subject suffering from Canavan disease into iPSCs, introducing a functional ASPA gene in the reprogrammed or converted iPSCs to obtain genetically corrected iPSCs which express a functional ASPA, and differentiating the genetically corrected iPSCs into neural precursor cells, including NPCs, glial progenitor cells, and OPCs. Alternatively, the method includes the steps of reprogramming or converting somatic cells isolated from a subject suffering from Canavan disease into iPSCs, differentiating the iPSCs into neural precursor cells, including NPCs, glial progenitor cells, and OPCs, and introducing a functional ASPA gene in the precursor cells to obtain genetically corrected precursor cells which express a functional ASPA.

In various embodiments of this disclosure, the somatic cells include but are not limited to fibroblasts, blood cells, urinary cells, adipocytes, keratinocytes, dental pulp cells and other easily accessible somatic cells. In some embodiments, the somatic cells isolated from the subject suffering from Canavan disease are converted into iPSCs in the presence of one or more reprogramming factors comprising OCT4, SOX2, KLF4, LIN28, p53 shRNA and MYC (such as c-MYC and L-MYC). In some embodiments, the reprogramming is carried out via episomal reprogramming or viral transduction.

In various embodiments of this disclosure, a functional ASPA includes the wild type ASPA or an ASPA comprising one or more mutations that do not substantially decrease the enzymatic activities of ASPA compared to wild type ASPA. In some embodiments, a functional ASPA includes R132G ASPA.

### BRIEF DESCRIPTION OF THE DRAWINGS

FIG. 1 illustrates a cell-based therapy for Canavan disease using human iPSC-derived NPCs and OPCs as disclosed herein.

FIGS. 2A-2D are plasmid maps of LV-EF1a-hASPA_R132G (2A), AAVS1-EF1a-ASPA-CD19t (2B), AAVS1-TALEN-L (2C), and AAVS1-TALEN-R (2D).

FIGS. 3A-3E show characterization of the ASPA iNPCs. FIG. 3A: The iPSC lines used in the study. FIG. 3B: The ASPA iNPC release test results. The ASPA activity was expressed as the increase of aspartic acid in nmol per mg cell lysates per hour at 37° C. % NPC was determined as the percent of CD133⁺SSEA4⁻ cells in the ASPA iNPCs by FACS. % residual iPSCs was determined as the percent of SSEA4⁺ cells by FACS or the percent of REX1⁺ cells by RT-qPCR. FIG. 3C: Immunostaining of ASPA iNPCs for the NPC markers NESTIN and SOX1. Scale bar: 50 μm. FIG. 3D: RT-PCR analysis of ASPA iNPCs for the expression of the NPC markers SOX1 and PAX6 and lack of expression of the pluripotency factors OCT4 and NANOG. ACTIN was included as a loading control. FIG. 3E: Flow cytometry analysis to determine the percentage of CD133⁺SSEA4⁻ NPC population and the residual SSEA4⁺ iPSC population in ASPA iNPCs. Isotype IgG was included as the negative control.

FIGS. 4A-4C show characterization of CD iPSCs. FIG. 4A: Expression of human pluripotency factors OCT4 and NANOG and the human ESC cell surface markers SSEA4, TRA-1-60 and TRA-1-81 in CD iPSCs. Three clones of CD iPSCs derived from each CD patient were included. Scale bar: 100 μm. FIG. 4B: Expression of pluripotency factors in CD iPSCs. RT-PCR analysis of endogenous OCT4, SOX2, and NANOG expression in CD iPSCs. Human H9 ESCs were included as the positive control (PC), and fibroblast cells were included as the negative control (NC). ACTIN was included as a loading control. FIG. 4C: Lack of residual reprogramming factors in CD iPSCs. PCR was performed to determine if there are any residual reprogramming factors in CD iPSCs. Fibroblast cells were included as the negative control (NC). The plasmid DNAs expressing individual reprogramming factors were included as the positive control (PC).

FIGS. 5A-5C show that CD iPSCs exhibited normal karyotype and ASPA mutations. FIG. 5A: CD iPSCs exhibit normal karyotype as reveal by G-banding. FIG. 5B: CD iPSCs contained patient-specific ASPA mutations as revealed by Sanger sequencing. The arrows indicate mutation sites. FIG. 5C: The R132G ASPA exhibits mildly increased ASPA activity per gene copy, compared to the wild type (WT) ASPA. The fold change is relative to the WT ASPA-transfected cells. ***p<0.001 by Student's t-test.

FIGS. 6A-6F show elevated ASPA activity and reduced NAA level in ASPA iNPC-transplanted CD (Nur7) mouse brains. FIG. 6A: Illustration of the injection sites in mouse brains. The bilateral injection sites were indicated in green dots. FIG. 6B: The ASPA iNPCs were distributed around the injection sites in the transplanted CD (Nur7) mouse brains three months after transplantation. The dot map of the human nuclear antigen (hNu) staining is shown. The injection sites were indicated by arrows. Scale bar: 1 mm. FIG. 6C: The ASPA iNPCs gave rise to neurons, astrocytes, and oligodendroglial lineage cells in the transplanted mouse brains. Three months after transplantation, the ASPA iNPC-transplanted brains were immunostained for hNu and the NPC marker PAX6, the neuronal marker NeuN, the astrocyte marker SOX9, and the oligodendroglial lineage marker OLIG2, respectively. The images from the subcortical white matter was shown. Scale bar: 50 μm. FIG. 6D: The percentage of hNu⁺ and the neural lineage marker⁺ cells in the transplanted brains. N=9 fields from 3 mice for each group. FIGS. 6E and 6F: Elevated ASPA activity (6E) and reduced NAA level (6F) in ASPA iNPC-transplanted CD (Nur7) mouse brains three months after transplantation. The NAA level was measured using NMR. The ASPA activity was measured by NMR and expressed as reduced NAA level per gram (g) of brain tissue within an hour (hr) (μmol/g/hr). Each dot represents the result from an individual mouse. n=6 mice for the WT, Het, and CD (Nur7) mice, 5 for the CD #59 ASPA iNPC, and 6 for the CD #60 ASPA iNPC and CD #68 ASPA iNPC-transplanted mice, respectively. Error bars are SE of the mean. ***p<0.001 by one-way ANOVA followed by Dunnett's multiple comparisons test for panels 6E-6F.

FIGS. 7A-7B show the cell fate of the ASPA iNPCs in different regions of transplanted CD (Nur7) mouse brains. FIG. 7A: The ASPA iNPCs were transplanted into CD (Nur7) mouse brains. Three months (3 m) after transplantation, the mouse brains were harvested and immunostained for hNu the NPC marker PAX6, the neuronal marker NeuN, the astrocyte marker SOX9, and the oligodendroglial lineage marker OLIG2, respectively. The ASPA iNPCs gave rise to neurons, astrocytes, and oligodendroglial lineage cells in the CD #59 ASPA iNPC, CD #60 ASPA iNPC and CD #68 ASPA iNPC-transplanted CD (Nur7) mouse brains. Scale bar: 50 μm. FIG. 7B: The percentage of hNu⁺ and the neural lineage marker+ cells in the different regions of transplanted brains. n=3 mice for each group.

FIGS. 8A-8F show the medium-treated CD (Nur7) mice exhibit deficits similar to the un-transplanted CD (Nur7) control mice. The medium for ASPA iNPCs was injected into CD (Nur7) mouse brains using the same coordinates and procedure as for cell transplantation and the treated mice were analyzed three months post-treatment. FIGS. 8A and 8B: Low ASPA activity (8A) and high NAA level (8B) in medium-treated CD (Nur7) mouse brains three months after transplantation. The ASPA activity was expressed as reduced NAA level per gram of brain tissue in an hour (μmol/g/hr). The data for the WT, Het and CD (Nur7) mice from FIGS. 6E and 6F were included here as controls. Each dot represents the result from an individual mouse for panels 8A & 8B. n=6 mice for WT, Het, and CD (Nur7) mice, respectively, and 4 for medium-treated mice. FIGS. 8C and 8D: Vacuolation in brains of medium-treated CD (Nur7) mice as revealed by H&E staining. The data for the WT, Het and CD (Nur7) mice from FIGS. 9A and 9B were included here as controls. Scale bar: 2,000 μm for 8C and 500 μm for 8D. FIGS. 8E and 8F: Deficit of motor function in medium-treated CD (Nur7) mice three months after transplantation as revealed by rotarod (8E) or grip strength (GS, 8F) test. Each dot represents the result from an individual mouse for panels 8E & 8F. The data for the WT, Het and CD (Nur7) mice from FIGS. 10D and 10E were included here as controls. n=8 mice for WT, Het, CD (Nur7) mice and medium-treated CD (Nur7) mouse.

FIGS. 9A-9C show reduced vacuolation in the ASPA iNPC-transplanted CD (Nur7) mouse brains. FIG. 9A: Reduced vacuolation in brains of the ASPA iNPC-transplanted CD (Nur7) mice three months after transplantation as revealed by H&E staining. Three whole brain sagittal sections of one mouse from each group are shown. The heterozygous (Het) mice were included as the positive control and the homozygous CD (Nur7) mice as the negative control. Scale bar: 2,000 μm. FIG. 9B: Enlarged H&E images of the subcortical white matter, the brain stem and the cerebellum are shown. Scale bar: 500 μm. FIG. 9C: Quantification of the vacuolation area in the subcortical, the brain stem, and the cerebellum white matter. n=3 mice per group. Error bars are SE of the mean. *p<0.05, **p<0.01, and ***p<0.001 by one-way ANOVA followed by Dunnett's multiple comparisons test.

FIGS. 10A-10E show improved myelination and motor function ASPA iNPC-transplanted CD (Nur7) mice. FIG. 10A: Improved myelination in the ASPA iNPC-transplanted CD (Nur7) mouse brains three months after transplantation. Improved myelination was shown by electron microscope and revealed by increased number of intact myelin sheaths and enhanced thickness of myelin sheaths in brains of the transplanted mice, compared to control CD (Nur7) mice. The subcortical white matter was processed and analyzed. Scale bar: 1 μm. FIGS. 10B and 10C: Quantification showing increased number of intact myelin sheaths (10B) and enhanced thickness of myelin sheaths as revealed by reduced G ratio (10C) in brains of the ASPA iNPC-transplanted miceCD (Nur7) mice, compared to that in control CD (Nur7) mice. n=15 myelin sheaths from one mouse brain for each group. 3 transplanted brains (one brain for each line) were analyzed. Error bars are SE of the mean. FIGS. 10D and 10E: Improved motor function in ASPA iNPC-transplanted CD (Nur7) mice three months after transplantation revealed by rotarod (10D) and grip strength (GS, 10E) tests. Each dot represents the result from an individual mouse. n=8 mice for the WT, Het, and CD (Nur7) mice, 23, 25, and 25 for the CD #59 ASPA iNPC, CD #60 ASPA iNPC, and CD #68 ASPA iNPC-transplanted mice, respectively, for panels 10D and 10E. *p<0.05, **p<0.01, and ***p<0.001 by one-way ANOVA followed by Tukey's multiple comparisons test for panels 10B & 10C and by Dunnett's multiple comparisons test.

FIG. 11 shows myelination in the ASPA iNPCs and ASPA iOPC-transplanted CD (Nur7) mouse brains. Three months after transplantation, the mouse brains were harvested and immunostained for the myelination marker MBP. The whole brain sagittal sections are shown in the left panels. The red arrows indicate areas in which the myelination extent is different in the CD (Nur7) mice, compared to that in the Het or transplanted mice. Enlarged images of the subcortical white matter, the brain stem and the cerebellum are shown in the right panels. Scale bar: 1,000 μm for whole brain sagittal section images, and 50 μm for enlarged images.

FIGS. 12A-12E show the cell fate of the ASPA iNPCs in transplanted CD (Nur7) mouse brains six months after transplantation. FIG. 12A: The ASPA iNPCs gave rise to neurons, astrocytes, and oligodendroglial lineage cells in the CD #68 ASPA iNPC-transplanted CD (Nur7) mouse brains. Six months after transplantation, the ASPA iNPC-transplanted brains were immunostained for hNu and the NPC marker PAX6, the neuronal marker NeuN, the astrocyte marker SOX9, and the oligodendroglial lineage marker OLIG2, respectively. Scale bar: 50 μm. FIG. 12B: The percentage of hNu⁺ and the neural lineage marker+ cells in the different regions of transplanted brains. n=3 mice for each marker. Error bars are SE of the mean. FIG. 12C: The percentage of hNu⁺ and the neural lineage marker+ cells in the CD #68 ASPA iNPC-transplanted CD (Nur7) mouse brains three and six months after transplantation. The 3-month quantification data from FIG. 6D was included here for comparison. n=9 fields from 3 mice for each group. Scale bar: 50 μm. FIGS. 12D and 12E: Low mitotic index in ASPA iNPC-transplanted CD (Nur7) mouse brains as revealed by hNu and Ki67 co-staining three (12D) or six months (12E) after transplantation. The images from the corpus callosum and the brain stem are shown. Scale bar: 50 μm.

FIGS. 13A-13K show sustained efficacy of ASPA iNPCs in transplanted CD (Nur7) mice 6 months after transplantation. FIGS. 13A and 13B: Elevated ASPA activity (13A) and reduced NAA level (13B) in ASPA iNPC-transplanted CD (Nur7) mouse brains six months after transplantation. The ASPA activity and NAA level was measured using NMR as described earlier. n=4 mice for each group. FIGS. 13C-13E: Reduced vacuolation in brains of ASPA iNPC-transplanted CD (Nur7) mouse brains as revealed by H&E staining. Quantification is shown in panel 13C, and enlarged H&E images are shown in panel 13E. n=3 mice for each group. Scale bar: 2,000 μm for 13D and 500 μm for 13E. FIGS. 13F and 13G: Improved motor function in ASPA iNPC-transplanted CD (Nur7) mice six months after transplantation, as revealed by rotarod (13F) and grip strength (GS, 13G) tests. n=8 mice for WT, Het and CD (Nur7) mice, respectively, 6 for CD #59 ASPA iNPC, 8 for CD #60 ASPA iNPC, and 7 for CD #68 ASPA iNPC-transplanted mice. FIG. 13H: Life span of ASPA iNPC-transplanted CD (Nur7) mice. The survival of the transplanted mice was monitored over 10 months. The CD (Nur7) mice were included as the negative control and the WT/Het mice as the positive control. n=20 for CD, 14 for WT/Het, and 20 for the transplanted mice. FIGS. 13I and 13J: Low mitotic index in ASPA iNPC-transplanted CD (Nur7) mouse brains as revealed by hNu and Ki67 co-staining three (13I) or six months (13J) after transplantation. The images from the subcortical white matter was shown. Scale bar: 50 μm. FIG. 13K: The percentage of the hNu⁺Ki67⁺ cells out of total hNu⁺ cells in the transplanted brains. n=9 fields from 3 mice for each group. Error bars are SE of the mean. ***p<0.001 by one-way ANOVA followed by Dunnett's multiple comparisons test for panels 13A-13C, 13F-13G. ***p<0.001 by Log-rank test between CD (Nur7) mice and ASPA iNPC-transplanted mice for panel 13H. *p<0.05 by one-way ANOVA followed by Dunnett's multiple comparisons test for panel 13K.

FIGS. 14A-14I show characterization of ASPA iOPC. FIG. 14A: Schematic for introducing the WT ASPA gene into the AAVS1 locus in CD iPSCs by TALEN-mediated gene editing. FIG. 14B: Flow cytometry analysis of the CD #68T-13 ASPA iPSCs using CD19-specific antibody. The isotype IgG was included as the negative control (blue). The ASPA-T2A-CD19t-positive cells were show in red. FIG. 14C: Immunostaining of the CD #68T-13 ASPA iOPCs for the oligodendroglial lineage markers OLIG2 and O4. FIG. 14D: Flow cytometry analysis of the ASPA iOPCs using CD140a-specific antibody. The isotype IgG was included as the negative control. FIG. 14E: Lack of residual SSEA4-positive iPSCs in ASPA iOPCs as revealed by flow cytometry. The isotype IgG was included as the negative control, which showed similar SSEA4⁺ population to that of SSEA4 antibody-based flow. FIG. 14F: The ASPA iOPCs displayed potent ASPA enzymatic activity, compared to the control CD iOPCs. n=3 replicates. **p<0.01 by Student's t-test (two tailed). FIG. 14G: Dot map shows widespread distribution of the transplanted ASPA iOPCs in CD (Nur7) mouse brains by immunostaining for hNu three months after transplantation. FIG. 14H: Co-staining of the transplanted CD (Nur7) mouse brains for human nuclear antigen hNu and the oligodendroglial lineage marker OLIG2, the neuronal marker NeuN, or the astrocyte marker SOX9, respectively. The images from the subcortical white matter was shown. FIG. 14I: The percentage of the hNu⁺NeuN⁺, hNu⁺SOX9⁺, and hNu⁺OLIG2⁺ population in the ASPA iOPC-transplanted (Nur7) mouse brains. n=9 fields from 3 mice for each group. Scale bar: 100 μm for 14C, 2,000 μm for 14G and 50 μm for 14H. Error bars are SE of the mean for panels 14F and 14I.

FIGS. 15A-15D show characterization of ASPA iOPCs. FIG. 15A: The CD #68T-13 ASPA iOPCs exhibited normal karyotype. FIG. 15B: Co-staining of the transplanted CD (Nur7) mouse brains for human nuclear antigen hNu and the oligodendroglial lineage marker OLIG2, the neuronal marker NeuN, or the astrocyte marker SOX9, respectively. The corpus callosum and the brain stem regions were shown. FIG. 15C: The percentage of the hNu⁺NeuN⁺, hNu⁺SOX9⁺, and hNu⁺OLIG2⁺ population in the different regions of ASPA iOPC-transplanted (Nur7) mouse brains. n=3 mice for each group. FIG. 15D: The ASPA iOPCs showed low mitotic index in transplanted mouse brains as revealed by hNu and Ki67 co-staining. The corpus callosum and the brain stem regions are shown. Scale bar: 50 μm. Error bars are SE of the mean.

FIGS. 16A-16H show the ASPA iOPCs rescued multiple deficits in CD (Nur7) mice. FIGS. 16A and 16B: Elevated ASPA activity (16A) and reduced NAA level (16B) in ASPA iOPC-transplanted CD (Nur7) mouse brains three months after transplantation measured by NMR. The ASPA activity was expressed as reduced NAA level per gram of brain tissue in an hour (μmol/g/hr). The same data for the WT, Het and CD (Nur7) mice from FIGS. 6E and 6F as were included here as controls. Each dot represents the result from an individual mouse for panels 16A & 16B. n=6 mice for WT, Het, and CD (Nur7) mice, respectively, and 5 for the CD #68T ASPA iOPC-transplanted mice. FIGS. 16C-16E: Reduced vacuolation in brains of ASPA iOPC-transplanted CD (Nur7) mouse brains as revealed by H&E staining. Quantification is shown in panel 16C, and enlarged H&E images are shown in panel 16E. n=9 fields from 3 mice for panel 16C. Scale bar: 2,000 μm for 16D and 500 μm for 16E. FIGS. 16F and 16G: Improved motor function in ASPA iOPC-transplanted CD (Nur7) mice three months after transplantation as revealed by rotarod (16F) or grip strength (GS, 16G) test. Each dot represents the result from an individual mouse for panels 16F & 16G. n=8 mice for WT, Het, and CD (Nur7) mice, respectively, and 7 for the CD #68T ASPA iOPC-transplanted mice. The same data for the WT mice from FIG. 6C were included here as a control. FIG. 16H: The ASPA iOPCs showed low mitotic index in transplanted mouse brains as revealed by hNu and Ki67 co-staining and the low percentage of the hNu⁺Ki67⁺ cells out of total hNu⁺ cells. The images from the subcortical white matter was shown. n=9 fields from 3 mice for panel 16H. Error bars are SE of the mean. *p<0.05, **p<0.01 and ***p<0.001 by one-way ANOVA followed by Dunnett's multiple comparisons test for panels 16A-16C, 16F-16G. ns stands for not statistically significant (p>0.05).

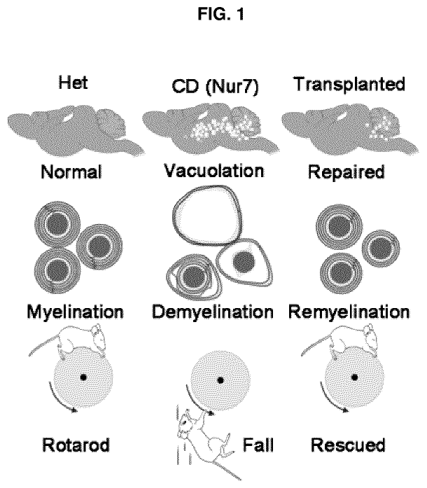

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

Disclosed herein is a cell-based therapy for Canavan disease (CD) using human iPSC-derived NPCs and OPCs. CD is a fatal leukodystrophy caused by mutation of the aspartoacylase (ASPA) gene, which leads to deficiency in ASPA activity, accumulation of the substrate N-acetyl-L-aspartate (NAA), demyelination and spongy degeneration of the brain. There is neither a cure nor a standard treatment for this disease. Disclosed herein is a human iPSC-based cell therapy developed for CD. A functional ASPA gene is introduced into patient iPSC-derived neural progenitor cells (iNPCs) or oligodendrocyte progenitor cells (iOPCs) via lentiviral transduction or TALEN-mediated genetic engineering to generate ASPA iNPCs or ASPA iOPCs. As demonstrated in the working examples, after stereotactic transplantation into a CD (Nur7) mouse model, the engrafted cells were able to rescue major pathological features of CD, including deficient ASPA activity, elevated NAA levels, extensive vacuolation, defective myelination, and motor function deficits, in a robust and sustainable manner. Moreover, the transplanted mice exhibited much prolonged survival. These genetically engineered patient iPSC-derived cellular products are promising cell therapies for CD. This study has the potential to bring effective cell therapies, for the first time, to Canavan disease children who have no treatment options. The approach established in this study could also benefit many other children who have deadly genetic diseases that have no cure.

Stem cell technology holds great promise for the treatment of intractable human diseases. Several clinical trials are ongoing using cells derived from human embryonic stem cells (hESCs) or human induced pluripotent stem cells (hiPSCs) [5]. iPSCs could provide an autologous and expandable donor source for the generation of specific somatic cell types and tissues from individual patients [6]. Furthermore, patient-specific iPSCs are tailored to specific individuals, and therefore could reduce the potential for immune rejection. Neural progenitor cells (NPCs) have been used in clinical trials and shown a favorable safety profile [7]. The high expandability and short differentiation time [8] make iPSC-derived NPCs (iNPCs) a desirable cell source for cell therapy.

The combination of gene therapy with cell therapy provides tremendous hope for a variety of genetic disorders. The therapeutic combination of patient-specific iPSCs with gene therapy provides an opportunity to correct gene defects in vitro, and these genetically-repaired iPSCs can then be appropriately characterized to ensure that the genetic correction is precise, thereby reducing safety concerns associated with direct gene therapy, such as random gene insertions.

Considerable interest has been aroused in generating iPSCs from patients of neurodegenerative diseases since the breakthrough development of the iPSC technology. These patient-specific iPSCs offer many opportunities for disease modeling, drug discovery, and cell replacement therapy. On the other hand, extensive efforts have been made to develop and optimize methods to differentiate pluripotent stem cells into different neural lineages. These methods allow the generation of neural cell types from genetically corrected iPSCs for cell replacement therapy.

Demyelinating diseases stand out as a particularly promising target for cell-based therapy of central nervous system disorders because remyelination can be achieved with a single cell type, and transplanted myelinogenic cells do not need to integrate into complex neuronal networks. Indeed, the myelinogenic potential of rodent and human pluripotent stem cell derivatives have been well documented in various animal models. The widespread myelination that can be observed in animal models supports the idea that cell therapy provides a potential therapeutic approach in dysmyelinating and demeylinating diseases.

Because CD is a demyelination disease with oligodendrocyte loss in the brain of CD patients, oligodendrocyte progenitor cells (OPCs), the precursor cells of oligodendrocytes, could also be a good candidate for CD cell therapy [9]. OPCs have been successfully derived from human iPSCs [10]. They are highly migratory after intracerebral engraftment, and can differentiate into oligodendrocytes and myelinate dysmyelinated loci throughout the brain [10a, 10b, 11].

As disclosed herein, iPSC-based cell therapy approach is combined with gene therapy approach to generate genetically-corrected patient iPSCs that express a functional ASPA gene (ASPA iPSCs). Subsequently, the ASPA iPSCs are differentiated into neural precursor cells, including NPCs, glial progenitor cells, oligodendroglial progenitor cells, and the therapeutic potential thereof is assessed in an immune-deficient Canavan disease mouse model.

Thus, disclosed herein is a method of treating Canavan disease in a subject. The method combines patient-specific iPSCs with gene therapy to develop genetically-corrected patient iPSCs that express a functional ASPA gene. The corrected ASPA iPSCs were differentiated into NPCs or OPCs. Alternatively, genetic correction can occur at the NPCs or OPCs level, that is, the iPSCs derived from a patient are differentiated into NPCs or OPCs, and then a functional ASPA gene is introduced into the NPCs or OPCs to generate genetically-corrected NPCs or OPCs. The ability of these neural precursors to alleviate the disease phenotypes of CD was tested in a CD mouse model, as demonstrated in the working examples. Also, the preclinical efficacy for NPCs or OPCs derived from genetically corrected patient iPSCs to serve as a therapeutic candidate for CD is demonstrated in the working examples.

Also disclosed herein are GMP-compatible processes for human iPSC derivation, expansion, and differentiation. In certain embodiments, the iPSCs were generated from CD patients and the CD iPSCs were differentiated into iNPCs using GMP-compatible processes established herein. A functional ASPA gene was introduced into CD iNPCs by lentiviral transduction. In some embodiments, the functional ASPA gene includes one or more mutations which do not substantially reduce the ASPA activities. For example, the functional ASPA encompassed by this disclosure includes R132G ASPA. The resultant ASPA iNPCs were transplanted into the brains of an immunodeficient CD (Nur7) mouse model. The efficacy and preliminary safety of the transplanted ASPA iNPCs were evaluated. In certain embodiments, a functional ASPA gene was introduced into a defined locus in CD iPSCs by TALEN-mediated gene editing. These gene-edited iPSCs were further differentiated into OPCs. The resultant ASPA iOPCs were also transplanted into CD (Nur7) mouse brains to determine their efficacy and preliminary safety.

CD is a devastating neurological disease that has neither a cure nor a standard treatment [23]. In this study, the human iPSC-based cell therapeutic candidates are established for CD. To facilitate the transfer of the cell therapeutic candidates to the clinic, GMP-compatible processes were first established for human iPSC derivation, expansion and differentiation. Then the iPSCs were generated from CD patient fibroblast cells and these iPSCs were differentiated into iNPCs using the GMP-compatible processes established. To reconstitute ASPA activity which is deficient in both CD patients and mouse models, ASPA iNPCs were developed by introducing a functional ASPA gene through lentiviral transduction. The ASPA iNPCs were transplanted into CD (Nur7) mouse brains. As demonstrated in the working examples, these cells were able to improve the disease symptoms dramatically, as revealed by increased ASPA activity, decreased NAA levels, substantially reduced spongy degeneration in various brain regions, and rescued motor functions of the transplanted mice. The therapeutic effect is long-lasting, showing no diminishing effect by 6 months compared to 3 months post-transplantation. Moreover, the transplanted CD (Nur7) mice exhibited much prolonged survival.

As an alternative strategy to introducing a functional ASPA gene by lentiviral transduction at the iNPC stage, a functional ASPA gene such as a wild type was introduced together with a truncated CD19 (CD19t) into the AAVS1 safe harbor site in CD iPSCs through TALEN-mediated gene editing. The CD19t sequence has been used in a previous clinical trial and confirmed to be safe [24]. The CD19t tag provides a cell surface marker for in vivo tracking of transplanted cells in patient brains by flow cytometry and immunohistochemistry approaches and can induce cell elimination through antibody-dependent cellular cytotoxicity (ADCC) in case of adverse tumorigenic events [24-25]. TALEN-based editing was chosen for introducing a functional or wild type ASPA gene into CD iPSCs to generate the ASPA iOPC cell product because of the low off-target activity associated with TALEN [26]. Indeed, the whole genome sequencing revealed no off-target effects in the top 99 potential off-target sites. The TALEN-edited ASPA iPSCs were differentiated into iOPCs using an established protocol [10d, 11]. After being transplanted into CD (Nur7) mouse brains, these cells showed an ability to rescue the CD phenotype that was comparable to that of ASPA iNPCs. Moreover, the ASPA iOPCs had better migration and more than 80% transplanted ASPA iOPCs went to the oligodendroglial lineage. Importantly, no tumorigenesis or other adverse effect was observed in mice transplanted with either the ASPA iNPCs or the ASPA iOPCs. These results indicate that the ASPA iNPCs and the ASPA iOPCs both have the potential to serve as cell therapy candidates for CD.

Great efforts have been directed toward therapeutic development for CD. While most other approaches resulted in limited functional recovery, gene therapy seems a promising clinical option for CD [23b]. When the WT human ASPA gene was delivered into brains of CD animal models by recombinant adeno-associated virus (rAAV), encouraging results were seen [3-4, 27]. However, the early clinical trial using AAV to deliver the ASPA gene into CD patient brains was unable to reach the desired therapeutic efficacy, although the safety profile was good [3]. Recent studies showed that knockdown of the neuronal NAA-synthesizing enzyme Nat8I by antisense oligonucleotide or AAV-delivered shRNA to reduce NAA level improved disease phenotypes in ASPA^{nur7/nur7} mice [28], suggesting that targeting Nat8I could be a candidate approach to treat CD, although how to achieve sustained efficacy using this approach remains to be addressed.

Compared to direct gene therapy, the combined cell and gene therapy approach used in this study allowed extensive in vitro characterization of the genetically modified cells before applying these cells to in vivo study. The ASPA iNPCs were examined for transgene copy number and all 6 ASPA iNPC lines had less than 5 copies of the transgene. The ASPA iPSCs that underwent TALEN-mediated gene editing were subjected to whole genome sequencing to make sure there were no adverse off-target effects before differentiation and transplantation. Furthermore, the lentivirus or TALEN-introduced ASPA transgene are likely more stable because of integration events, therefore allowing sustained ASPA activity in the host brains, unlike AAV-mediated transgene delivery which is episomal, thus can have more transient expression. The patient iPSC-derived autologous cellular products can also avoid potential immunogenicity associated with the AAV vector [29], and have the added benefit of regenerative potential linked to cell therapy [5b].

NPCs have been used in clinical trials and shown a favorable safety profile [7a-d]. NPCs isolated from human fetal brains have been transplanted into Pelizaeus-Merabacher disease (PMD) patient brains and exhibited long-term safety after 5 years of follow up [7c, 30]. No tumors or other long-term adverse effects were observed [7c]. Besides the favorable safety profile, the expandability and short manufacturing protocol make iNPCs a relatively economic and accessible cell source for cell therapy.

OPCs are another desirable cell therapy candidate for leukodystrophies including Canavan disease [9, 31]. This study and previous studies [10b, 32] have shown that OPCs can migrate widely after intracerebral transplantation, rendering OPCs a desired vector for widespread delivery. Moreover, it has been shown that the transplanted OPCs can differentiate into oligodendrocytes and myelinate dysmyelinated loci throughout the brain [10b, 11, 32]. In this study, it is shown that the ASPA iOPCs can migrate out of the injection sites, and rescue disease phenotypes dramatically in a leukodystrophy mouse model. However, compared to iNPCs, the differentiation protocol for iOPCs is more complex (requiring multiple growth factors), more time-consuming and costly. It takes about 70 days or more to differentiate from human iPSCs to iOPCs [10a, 10c], whereas differentiation from human iPSCs to iNPC only needs 8 days [8]. Moreover, the iNPCs are of high purity and can be easily expanded to produce enough cells for human applications [30]. The current protocol for iOPC differentiation can only produce limited number of cells and iOPCs are not as easy to maintain and expand. Further optimized protocol for iOPC differentiation with shorter differentiation time, simpler procedure with less expensive reagents, and higher differentiation efficiency may facilitate the application of iOPCs into the clinic.

Although the ASPA iNPCs did not migrate in the brain after transplantation, they were able to rescue the disease phenotypes in a robust and sustainable manner. One explanation for these unexpected results is because NAA travels in the brain through an intercompartmental cycling via extracellular fluids, between its anabolic compartment in neurons and catabolic compartment in oligodendrocytes [33] or transplanted ASPA iNPCs in this case. After NAA is released from neurons, it can move to the transplanted cells that have ASPA activity through a concentration gradient, therefore leading to widespread reduction of NAA level, and consequently extensive rescue of spongy degeneration and myelination defect in the brain.

Unlimited source of cells derived from iPSCs and the low risk of immune rejection associated with autologous cell transplantation render human iPSC-based autologous cellular products great potential for regenerative medicine [5b]. Indeed, the first clinical study using human iPSC-based product was initiated in 2014, in which autologous retinal pigment epithelium (RPE) sheets derived from patient's own iPSCs were transplanted back to the patient. This treatment has resulted in favorable outcome, halting macular degeneration in the absence of anti-VEGF drug administration [34].

Despite the huge advantage associated with human iPSC-derived cellular products, there remain issues related to iPSC-based cell therapy, including teratoma formation and high cost of individualized cell products. To address the safety concern associated with potential development of teratoma from iPSC products, an SOP that allows efficient and reproducible differentiation of iPSCs into iNPCs with undetectable residual iPSCs was developed. Whether there were any residual iPSCs in ASPA iNPCs was tested using both FACS analysis and RT-qPCR assay and a stringent release specification was set for the ASPA iNPC products. The residual iPSCs in all six ASPA iNPC products were below the detection limit for both FACS and RT-qPCR analyses. Furthermore, continuous monitoring of the ASPA iNPC-transplanted mice for up to 10 months and the ASPA iOPC-transplanted mice for 3 months revealed no sign of tumorigenesis. These results suggest the preclinical safety of our cellular products.

The use of autologous iPSCs as the source of cell therapy products comes at high cost. Ideally, an off-the-shelf allogenic product would address this concern. The use of allogeneic iPSCs, in which a single lot of cells could be used to treat multiple patients, would bring down the cost for iPSC-based cell product manufacturing. However, this would come at the price of immune rejection caused by HLA mismatching and, thus, poses a major challenge for allogeneic transplantation. The rejection issue has typically been addressed through immunosuppression, which has been effective but can itself be costly and its serious side effects for long term application [35] would further complicate the management of these CD patients. The approach taken in Japan by using iPSC stocks from HLA homozygous donors to cover most HLA haplotypes [36] would not likely be effective in CD which is associated with a diverse genetic background. An alternative approach manipulates the immune responses through gene editing to overcome immune rejection associated with allogeneic transplantation [37]. This approach has great potential to generate universal donor cells, but brings its own safety concerns, for example, the potential of increased tumorigenicity due to compromised immune surveillance. From the immunological point of view, autologous transplantation is ideal for cell therapy because these cells may avoid any potential immune-mediated complications. The cost of iPSC-based cell therapy manufacturing can be reduced with the availability of low-cost reagents [38], and de-risking of GMP manufacturing through the development of GMP-compatible processes as described in this study that are cost-effective and easily transferrable to GMP.

In one aspect, this disclosure relates to a method of treating Canavan disease in a subject. The method entails restoring ASPA enzymatic activities in the subject by expressing exogenous functional ASPA gene in the brain of the subject. In some embodiments, the functional ASPA gene is a wild type ASPA gene. In some embodiments, the functional ASPA gene has one or more mutations that do not result in a substantial reduction in ASPA activities. In some embodiments, the ASPA enzymatic activities are restored by transplanting ASPA NPCs or OPCs in the brain of the subject. These ASPA NPCs or OPCs serve as a source of the ASPA enzyme. As detailed in this disclosure, ASPA NPCs or OPCs can be derived from patient-specific iPSCs. For example, the method further includes the steps of reprogramming or converting somatic cells isolated from the subject suffering from Canavan disease into induced pluripotent stem cells (iPSCs), introducing functional ASPA gene in the reprogrammed or converted iPSCs to obtain genetically corrected iPSCs which express functional ASPA, and differentiating the genetically corrected iPSCs into neural precursor cells, including NPCs, glial progenitor cells and oligodendroglial progenitor cells. Alternatively, the method further includes the steps of reprogramming or converting somatic cells isolated from the subject suffering from Canavan disease into iPSCs, differentiating the iPSCs into neural precursor cells, including NPCs, glial progenitor cells and oligodendroglial progenitor cells, and then introducing a functional ASPA gene in the neural precursor cells to obtain genetically corrected neural precursor cells which express a functional ASPA. In some embodiments, the functional ASPA gene is a wild type ASPA gene. In some embodiments, the functional ASPA gene has one or more mutations that do not result in a substantial reduction in ASPA activities.

As used in this disclosure, a “functional” ASPA or ASPA gene means that the amino acid sequence or the nucleotide sequence of ASPA may contain one or more mutations; however, the activities of the mutated ASPA are not substantially reduced compared to the wild type ASPA. In some embodiments, a functional ASPA retains at least 75%, at least 80%, at least 85%, at least 90%, at least 95%, at least 100%, at least 105%, at least 110%, at least 115%, at least 120%, or at least 125% activities of the wild type ASPA.

In certain embodiments, the ASPA sequence is modified to create one or more mutations outside of the catalytic center of the ASPA such that the mutation(s) do not substantially decrease the ASPA activity. For example, R132G mutation in ASPA can be introduced. In certain embodiments, the mutated ASPA sequence is used in the clinic to track the transplanted cells during treatment such that any adverse events that are associated with the transplanted cells can be monitored. For example, if there is a tumor in the patient's brain, it can be monitored whether the tumor is arisen from the transplanted cells or from endogenous cells by using the mutated ASPA sequence. In certain embodiments, genomic DNA PCR or RT-PCR followed by restriction enzyme digestion are performed to track the transplanted cells. The R132G ASPA gives a different digestion pattern from the ASPA with the natural R132 residue. Alternatively, immunostaining using an antibody specific to the ASPA R132G form can be used to track the transplanted cells.

In certain embodiments, the one or more mutations are outside of the catalytic centers of ASPA (SEQ ID NO: 1):

MISCHIAEEH IQKVAIFGGT HGNELIGVFL
VKHWLENGAE IQRTGLEVKP FITNPRAVKK
CTRYIDCDLN RIFDLENLGK KMSEDLPYEV
RRAQEINHLF GPKDSEDSYD IIFDLHNTTS
NMGCTLILED SRNNFLIQMF HYIKTSLAPL
PCYVYLIEHP SLKYATTRSI AKYPVGIEVG
PQPQGVLRAD ILDQMRKMIK HALDFIHHEN
EGKEFPPCAI EVYKIIEKVD YPRDENGEIA
AIIHPNLQDQ DWKPLHPGDP MELILDGKTI
PLGGDCTVYP VFVNEAAYYE KKEAFAKTTK
LTLNAKSIRC CLH

ASPA binds one atom of Zn per monomer [50] and this metal is necessary for the enzyme reaction. The amino acid residues involved in Zn binding include His21, Glu24, and His116. The catalytic site can be composed of residues Arg63, Asn70, Arg71, Tyr164, Arg168, Glu178, and Tyr288. Residues Arg168 and Tyr288 may stabilize the binding of NAA to ASPA. Accordingly, other mutations outside of these regions, which do not substantially compromise the ASPA activities can be included.

hASPA-R132G nucleotide sequence (SEQ ID NO: 2), with the point mutations shown in bold and underlined. Specifically, mutation 394: A to G mutation changes Arg132 (AGG) to Gly132 (GGG); and mutation 735: a synonym mutation T to C keeps Pro245 (CCT) as Pro245 (CCC).

ATGACTTCTTGTCACATTGCTGAAGAACATATACAAAAGGT
TGCTATCTTTGGAGGAACCCATGGGAATGAGCTAACCGGA
GTATTTCTGGTTAAGCATTGGCTAGAGAATGGCGCTGAGA
TTCAGAGAACAGGGCTGGAGGTAAAACCATTTATTACTAA
CCCCAGAGCAGTGAAGAAGTGTACCAGATATATTGACTGT
GACCTGAATCGCATTTTTGACCTTGAAAATCTTGGCAAAA
AAATGTCAGAAGATTTGCCATATGAAGTGAGAAGGGCTCA
AGAAATAAATCATTTATTTGGTCCAAAAGACAGTGAAGAT
TCCTATGACATTATTTTTGACCTTCACAACACCACCTCTA
ACATGGGGTGCACTCTTATTCTTGAGGATTCCGGGAATAA
CTTTTTAATTCAGATGTTTCATTACATTAAGACTTCTCTG
GCTCCACTACCCTGCTACGTTTATCTGATTGAGCATCCTT
CCCTCAAATATGCGACCACTCGTTCCATAGCCAAGTATCC
TGTGGGTATAGAAGTTGGTCCTCAGCCTCAAGGGGTTCTG
AGAGCTGATATCTTGGATCAAATGAGAAAAATGATTAAAC
ATGCTCTTGATTTTATACATCATTTCAATGAAGGAAAAGA
ATTTCCTCCCTGCGCCATTGAGGTCTATAAAATTATAGAG
AAAGTTGATTACCCCCGGGATGAAAATGGAGAAATTGCTG
CTATCATCCATCCCAATCTGCAGGATCAAGACTGGAAACC
ACTGCATCCTGGGGATCCCATGTTTTTAACTCTTGATGGG
AAGACGATCCCACTGGGCGGAGACTGTACCGTGTACCCCG
TGTTTGTGAATGAGGCCGCATATTACGAAAAGAAAGAAGC
TTTTGCAAAGACAACTAAACTAACGCTCAATGCAAAAAGT
ATTCGCTGCTGTTTACATTAG

hASPA-R132G amino acid sequence (SEQ ID NO: 3), with the point mutation shown in bold and underlined:

MTSCHIAEEHIQKVAIFGGTHGNELTGVFLVKHWLENGAE
IQRTGLEVKPFITNPRAVKKCTRYIDCDLNRIFDLENLGK
KMSEDLPYEVRRAQEINHLFGPKDSEDSYDIIFDLHNTTS
NMGCTLILEDSGNNFLIQMFHYIKTSLAPLPCYVYLIEHP
SLKYATTRSIAKYPVGIEVGPQPQGVLRADILDQMRKMIK
HALDFIHHFNEGKEFPPCAIEVYKIIEKVDYPRDENGEIA
AIIHPNLQDQDWKPLHPGDPMFLTLDGKTIPLGGDCTVYP
VFVNEAAYYEKKEAFAKTTKLTLNAKSIRCCLH

In some embodiments, the somatic cells include but are not limited to fibroblasts, blood cells, urinary cells, adipocytes, keratinocytes, dental pulp cells, and other easily accessible somatic cells. In some embodiments, the somatic cells isolated from the subject suffering from Canavan disease are converted into iPSCs in the presence of one or more reprogramming factors comprising OCT4, SOX2, KLF4, LIN28, p53 shRNA and MYC (such as c-MYC and L-MYC). In some embodiments, the reprogramming is carried out via episomal reprogramming or viral transduction. It is within the purview of one skilled in the art to select a reprogramming technique to convert the patient somatic cells into iPSCs. In some embodiments, a functional ASPA gene is introduced into the reprogrammed iPSCs by transducing the reprogrammed iPSCs with a vector comprising the exogenous functional ASPA gene. It is within the purview of one of ordinary skill in the art to select a suitable vector and promoter to express the functional ASPA gene after transduction. In some embodiments, the functional ASPA gene is introduced by gene editing technology such as the CRISPR/Cas9 technology or TALEN-mediated genetic engineering.

In another aspect, this disclosure relates to a method of treating Canavan disease in a subject. The method entails the steps of reprogramming or converting somatic cells isolated from the subject suffering from Canavan disease into induced pluripotent stem cells (iPSCs), introducing a functional ASPA gene in the reprogrammed or converted iPSCs to obtain genetically corrected iPSCs which express a functional ASPA, differentiating the genetically corrected iPSCs into neural precursor cells such as NPCs and OPCs, and transplanting the neural precursor cells into the brain of the subject. In some embodiments, the method entails the steps of reprogramming or converting somatic cells isolated from the subject suffering from Canavan disease into induced pluripotent stem cells (iPSCs), differentiating the iPSCs into neural precursor cells such as NPCs or OPCs, introducing a functional ASPA gene in the neural precursor cells to obtain genetically corrected neural precursor cells which express a functional ASPA, and transplanting the genetically corrected neural precursor cells into the brain of the subject.

In some embodiments, the somatic cells include but are not limited to fibroblasts, blood cells, urinary cells, adipocytes, keratinocytes, dental pulp cells, and other easily accessible somatic cells. In some embodiments, the somatic cells isolated from the subject suffering from Canavan disease are converted into iPSCs in the presence of one or more reprogramming factors comprising OCT4, SOX2, KLF4, LIN28, p53 shRNA and MYC (such as c-MYC and L-MYC).

In some embodiments, the reprogramming is carried out via episomal reprogramming or viral transduction. It is within the purview of one skilled in the art to select a reprogramming technique to convert the patient somatic cells into iPSCs. The iPSCs converted from patient somatic cells contain one or more mutations in the ASPA protein. For example, some patients suffering from Canavan disease carry one or more mutations in the ASPA protein, such as A305E, E285A, or G176E mutation, resulting from a codon change of 914C>A, 854A>C, and 527G>A, respectively. Some Canavan disease patients may carry other mutations in different regions of the ASPA protein. Upon introducing a functional ASPA gene into the patient iPSCs, these iPSCs are genetically corrected to express an exogenous functional ASPA protein and exhibit ASPA enzymatic activities that are substantially the same as the wild type ASPA.

In some embodiments, a functional ASPA gene is introduced into the reprogrammed iPSCs by transducing the reprogrammed iPSCs with a vector such as a viral vector comprising an exogenous functional ASPA gene. It is within the purview of one of ordinary skill in the art to select a suitable vector and promoter to express a functional ASPA gene after transduction. For example, an exogenous functional ASPA gene can be introduced by transducing the patient iPSCs with a lentivirus comprising the functional ASPA gene. The ASPA gene mutation in Canavan disease patient iPSCs can also be corrected by gene editing technologies, such as the CRISPR/Cas9 technology or TALEN-mediated genetic engineering. The genetically corrected iPSCs are differentiated in vitro into neural precursor cells such as NPCs and OPCs, which express a functional ASPA. In some embodiments, the genetic correction occurs at the neural precursor cells level in a similar fashion. The CD patient iPSCs are differentiated into neural precursor cells, and then a functional ASPA gene is introduced to the neural precursor cells by transduction or gene editing, which techniques are known in the art.

In another aspect, this disclosure relates to a method of producing ASPA neural precursor cells such as NPCs and OPCs which serve as a source of the ASPA enzyme for treating Canavan disease. The ASPA neural precursor cells are derived from patient-specific iPSCs. The method includes the steps of reprogramming or converting somatic cells isolated from a subject suffering from Canavan disease into iPSCs, introducing a functional ASPA gene in the reprogrammed or converted iPSCs to obtain genetically corrected iPSCs which express a functional ASPA, and differentiating the genetically corrected iPSCs into neural precursor cells such as NPCs and OPCs. Alternatively, the method includes the steps of reprogramming or converting somatic cells isolated from a subject suffering from Canavan disease into iPSCs, differentiating the iPSCs into neural precursor cells such as NPCs and OPCs, and introducing a functional ASPA gene in the neural precursor cells to obtain genetically corrected neural precursor cells which express a functional ASPA.

In a related aspect, this disclosure relates to neural precursor cells such as NPCs and OPCs which express an exogenous functional ASPA gene produced by a process comprising the steps of reprogramming or converting somatic cells isolated from a subject suffering from Canavan disease into iPSCs, introducing afunctional ASPA gene in the reprogrammed or converted iPSCs to obtain genetically corrected iPSCs which express a functional ASPA, and differentiating the genetically corrected iPSCs into neural precursor cells. Alternatively, the process comprises the steps of reprogramming or converting somatic cells isolated from a subject suffering from Canavan disease into iPSCs, differentiating the iPSCs into neural precursor cells such as NPCs and OPCs, and introducing a functional ASPA gene in the neural precursor cells to obtain genetically corrected neural precursor cells which express a functional ASPA. As used herein, neural precursor cells include NPCs, glial progenitor cells and OPCs.

In some embodiments, the somatic cells include but are not limited to fibroblasts, blood cells, urinary cells, adipocytes, keratinocytes, dental pulp cells, and other easily accessible somatic cells. In some embodiments, the somatic cells isolated from the subject suffering from Canavan disease are converted into iPSCs in the presence of one or more reprogramming factors comprising OCT4, SOX2, KLF4, LIN28, p53 shRNA and MYC (such as c-MYC and L-MYC). In some embodiments, the reprogramming is carried out via episomal reprogramming or viral transduction. It is within the purview of one skilled in the art to select a reprogramming technique to convert the patient somatic cells into iPSCs. In some embodiments, a functional ASPA gene is introduced into the reprogrammed iPSCs by transducing the reprogrammed iPSCs with a vector comprising the exogenous functional ASPA gene or by genetic editing technology such as CRISPR or TALEN-mediated genetic engineering. It is within the purview of one of ordinary skill in the art to select a suitable vector and promoter to express the functional ASPA gene after transduction.

The terms “treat,” “treating,” and “treatment” as used herein with regards to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. In some embodiments, treating a condition means that the condition is cured without recurrence.

The terms “subject” and “patient” are used interchangeably in this disclosure. In some embodiments, the subject or patient suffers from Canavan disease. In some embodiments, the subject or patient is a mammal. In some embodiments, the subject or patient is a human.

The working examples below further illustrate various embodiments of this disclosure. By no means the working examples limit the scope of this invention.

### Example 1: Materials and Methods

The following materials and methods apply to the studies discussed in Examples 2-14 below unless otherwise specified.

CD iPSC production: The CD iPSCs were manufactured using an integration-free, xeno-free and feeder-free method by following the specific standard operation procedure (SOP) established in this study. Specifically, the CD patient fibroblasts CD59 (Coriell, GM00059), CD60 (Coriell, GM00060), CD68 (Coriell, GM04268), CD92 (ID 21282, Biobank code FFF0871992, Telethon), CD00 (ID 22217, Biobank code FFF0282000, Telethon) and CD01 (ID 22276, Biobank code FFF0082001, Telethon) were reprogrammed using episomal vectors expressing human OCT4, SOX2, KLF4, L-MYC, LIN28 and p53 shRNA (sh-p53) (Addgene plasmids pCXLE-hSK, pCXLEhUL, pCXLE-hOCT3/4-shp53-F, and pCXWB-EBNA1, Table 1) as described [12].

TABLE 1
List of Plasmids

| Plasmid Name | Vendor | CAT. # |
|---|---|---|
| pCXLE-hSK | Addgene | Cat# 27078 |
| pCXLE-hUL | Addgene | Cat# 27080 |
| pCXLE-hOct3/4-shp53-F | Addgene | Cat# 27077 |
| pCXWB-EBNA1 | Addgene | Cat# 37624 |
| hAAVS1 TALEN Right | Addgene | Cat# 52342 |
| hAAVS1 TALEN Left | Addgene | Cat# 52341 |
| AAVS1-CAG-hrGFP | Addgene | Cat# 52344 |

The cells electroporated with the reprogramming vectors using 4D Nucleofector (Lonza) were seeded onto plates coated with recombinant human Laminin-521 matrix (Thermo Fisher, A29249) and maintained in Essential 8 (E8) medium (Thermo Fisher, A1517001), a xeno-free medium. The iPSC clones were picked around day 20 and expanded in E8 medium. For immunostaining, the iPSCs were passaged and seeded on 12-well Laminin-521-coated plates for 2 to 3 days. The resultant iPSC clones were ready for staining.

Differentiation of CD iPSCs into CD iNPCs: The CD iPSCs were differentiated into neural progenitor cells (iNPCs) on recombinant human Laminin-521-coated plates by following the SOP that was developed following an established protocol [8]. To start neural induction, the human iPSCs were dissociated into single cells, seeded onto Laminin-521-coated plate, and cultured in E8 medium. After 2 days, the cells were switched to Neural Induction Medium 1 (NIM-1) containing DMEM/F12 (Thermo Fisher, 11330032), 1×N2 (Thermo Fisher, 17502048), 1×B27 (Thermo Fisher, 12587010), 1×NEAA (Gibico, 11140076), 2 mM GlutaMAX (Thermo Fisher, 35050061), 0.1 μM RA (Sigma, R2625), 4 μM CHIR99021 (Cellagen Technology, C2447), 3 μM SB431542 (Peprocell, 04-0010-10), 2 μM Dorsomorphin (Sigma, P5499) and 10 ng/ml hLIF (Millipore Sigma, GF342). The cells were cultured in NIM-1 for 2 days, then switched to Neural Induction Medium 2 (NIM-2) containing DMEM/F12, 1×N2, 1×B27, 1×NEAA, 2 mM GlutaMAX, 0.1 μM RA, 4 μM CHIR99021, 3 μM SB431542, and 10 ng/ml hLIF with daily medium change for 5 days. The resultant iNPCs were dissociated and cultured in Neural Progenitor Maintenance Medium (NPMM) containing DMEM/F12, 1×N2, 1×B27, 2 mM GlutaMAX, 0.1 μM RA, 3 μM CHIR99021, 2 μM SB431542, 10 ng/ml EGF (PeproTech, 100-18b) and 10 ng/ml FGF (PeproTech, 100-15), with medium change every other day. The CD iNPCs were expanded and cells before passage 6 were used. For immunostaining, the dissociated single cells were seeded on Matrigel (Corning, 354230)-coated coverslip in 24 well plates for 2 to 3 days.

ASPA viral preparation and transduction: The cloned DNA that was used for genetic modification of CD iNPCs consists of the sequence of a functional human ASPA gene under the control of the constitutive human EF1α promoter. The human ASPA coding sequence was PCR-amplified using the ASPA cDNA clone MGC:34517 (IMAGE: 5180104) as the template. The ASPA cDNA was cloned into the pSIN lentiviral vector downstream of the EF1α promoter. The EF1α promoter and the ASPA cDNA fragments were subsequently PCR-amplified using the pSIN-ASPA as the template and subcloned into the self-inactivating pHIV7 lentiviral vector described previously [24, 39]. The resultant lentiviral vector was called LV-EF1α-hASPA. To track the transplanted cells in patient brains, a point mutation was created in the ASPA gene by changing the codon of Arginine (AGG) at amino acid residue 132 to that of Glycine (GGG). Arginine 132 was selected for mutation because it is located outside of the catalytic center of the ASPA protein. To package the ASPA-expressing lentivirus, the LV-EF1α-hASPA transgene vector, together with the VSV-G, REV and MDL packaging vectors were transfected into HEK 293T cells using the calcium phosphate transfection method as described previously [40]. Forty-eight hours after transfection, virus was harvested, concentrated by ultracentrifugation and stocked in −80° C. For lentiviral transduction, 1.5×10⁶ dissociated single NPCs were seeded in T25 flask and the viruses were added when the cells were attached. Then the ASPA iNPCs were lifted and expanded in suspension culture. The ASPA iNPCs before passage 6 were used for characterization and transplantation.

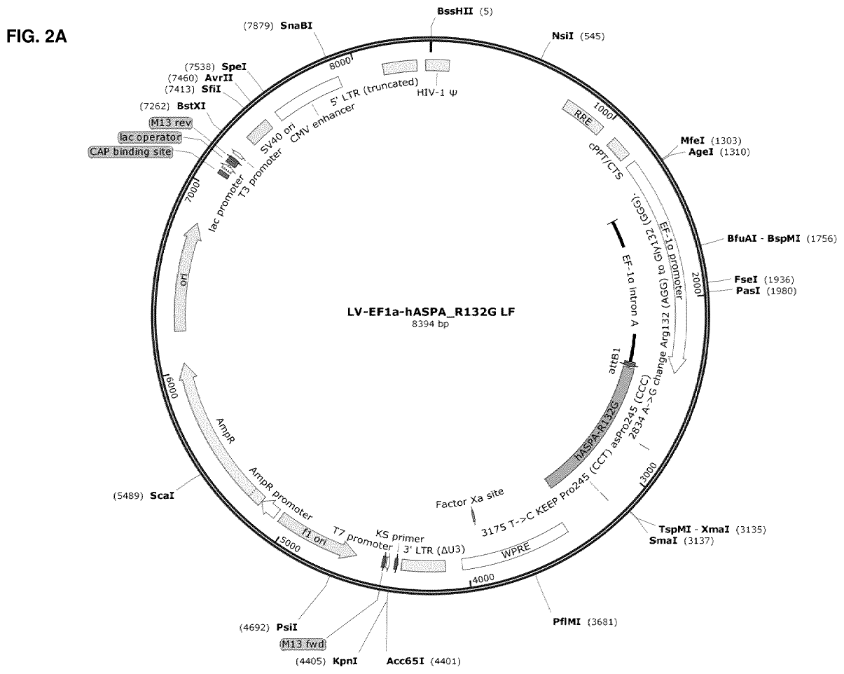

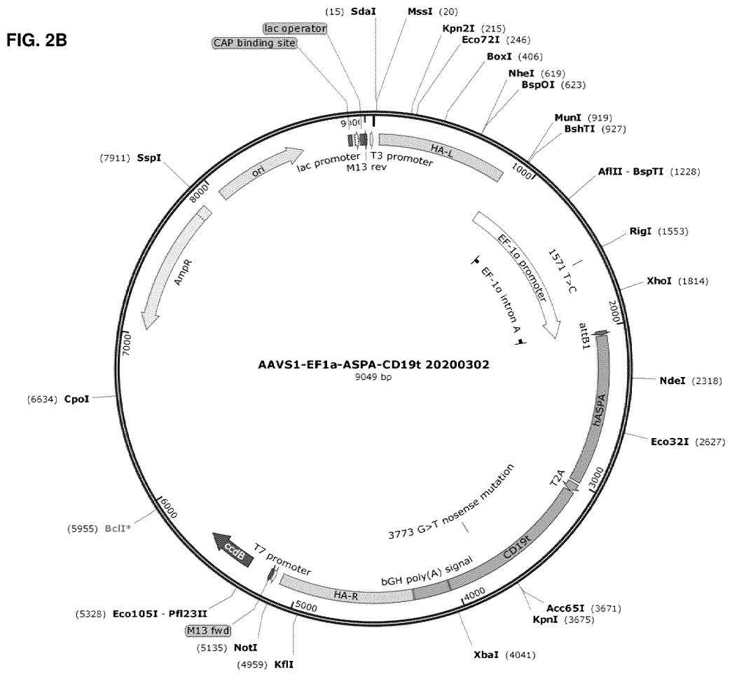

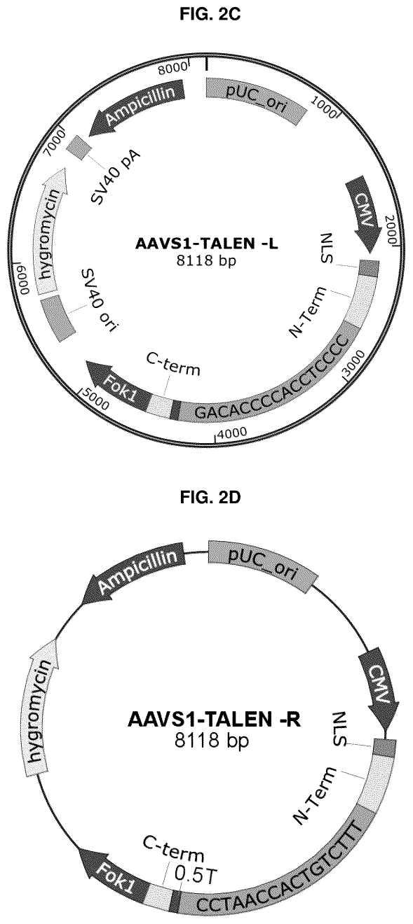

Generation of the ASPA-CD68 iPSCs using TALEN editing: The ASPA-CD68 iPSCs were generated by TALEN-mediated gene editing. The hAAVS1 TALEN left and right vectors were used for TALEN-mediated targeting of the AAVS1 locus as described [41]. The donor vector was constructed using the AAVS1-CAG-hrGFP vector by inserting the EF1α-ASPA-T2A-CD19t fragment between the AAVS1 left and right arm. The hAAVS1 TALEN left and right vectors and the donor plasmid were delivered via nucleofection into CD68 iPSCs. The transfected iPSCs were sorted by using the CD19 antibody and seeded as single cells. The single cell-derived clones were picked and screened by PCR. Three primers, AAVS1-Fwd, AAVS1-Rev and ASPA-Rev, were designed for genotyping of the iPSC clones. Three iPSC clones with homozygous insertion were chosen, expanded and stocked. The CD68T-13 iPSC clone was randomly selected from these three clones for further experiments. The hAAVS1 TALEN Right, hAAVS1 TALEN Left and AAVS1-CAG-hrGFP vectors were gifts from Dr. Su-Chun Zhang (Table 1). The TALEN-R sequence is: TTTCTGTCACCAATCC (SEQ ID NO: 4), and the TALEN-L sequence is: CCCCTCCACCCCACAG (SEQ ID NO: 5). FIG. 2 shows the plasmid maps of certain plasmids used herein.

Whole genome sequencing and TALEN off-target analysis: The genomic DNA from control CD iPSCs and TALEN-edited ASPA iPSCs were subjected to whole genome sequencing using the BGlseq 500 (MGI Tech). High quality genomic DNA was purified from the cells using Wizard® SV Genomic DNA Purification System (Promega, A2360) and quantified using Qubit 3.0 fluorometer. For sequencing library generation, the genomic DNA was fragmentated into sizes of 50-800 bp using ultrasound-based fragmentation (Covaris E220). The fragmented DNA were further selected with AMPure XP beads (Beckman Coulter, A63881) to enrich DNA of 100-300 bp, which were then repaired with a blunt ending enzyme and by addition of 3′ A overhang. A *T* tailed adapter was ligated to both ends of the DNA fragments and amplified by PCR (8 cycles). The PCR product was then denatured and annealed with a single strand bridging DNA that is reverse-complemented to both ends of the PCR product to generate single-strand circular DNA. The single-strand molecule was ligated using a DNA ligase. The excessive linear molecule was digested with the exonuclease. The DNA nanoballs (DNB) were then generated from the single-strand circular DNA according to the manufacturer's instruction (MGI Tech) and sequenced with BGISEQ-500 using pair-end 100 cycles. For each sample, coverage of over 30× was generated. The sequences of DNBs were base called using the base calling software Zebra call. Calling for variants were carried out with BWA [42] and GATK [43]. The structure variation was analyzed using breakDancer (http://www.nature.com/nmeth/journal/v6/n9/abs/nmeth.1363.html). The potential off-target sites of TALEN were predicted using a genome wide TALEN off-target site prediction tool TALENoffer [44]. A total of 100 sites including the target site and the tope 99 potential off-target sites were export from TALENoffer. The potential off-target sites were evaluated using whole genome sequencing. No mutation was found on any of these sites (Table 2).

TABLE 2
No mutation was detected in the top potential off-
target sites as revealed by WGS

| ID | Chromosome | Position 1 | Position 2 | Sequence 1 | Sequence 2 | Mutation |
|---|---|---|---|---|---|---|
| 1 | chr19 | 55627106 | 55627146 | TTTTCTGTCA | TTATCTGTCCCCTC | No |
| | | | | CCAATCCT | CACC (SEQ ID NO: | |
| | | | | (SEQ ID NO: 6) | 7) | |
| 2 | chr19 | 55627106 | 55627148 | TTTTCTGTCA | TTTTATCTGTCCCC | No |
| | | | | CCAATCCT | TCCA (SEQ ID NO: | |
| | | | | (SEQ ID NO: 6) | 8) | |
| 3 | chr3 | 33655954 | 33655990 | ATTTCTGTCA | TTGTCTTTCACTAA | No |
| | | | | AAAATCCT | TACT (SEQ ID NO: | |
| | | | | (SEQ ID NO: 9) | 10) | |
| 4 | chr22 | 51139502 | 51139538 | CTCCCCCCAC | TTTTCTGTCCCCAC | No |
| | | | | CCCCCAAA | TCCA (SEQ ID NO: | |
| | | | | (SEQ ID NO: | 12) | |
| | | | | 11) | | |
| 5 | chr5 | 126016079 | 126016113 | TACTCCCCAC | TCCCCTGCATCCA | No |
| | | | | CCCACAGA | ACAGT (SEQ ID NO: | |
| | | | | (SEQ ID NO: | 14) | |
| | | | | 13) | | |
| 6 | chr17 | 64789835 | 64789872 | TCCACCCTAC | TACCCTCCTCCCC | No |
| | | | | CTCCCAGC | ACAGT (SEQ ID NO: | |
| | | | | (SEQ ID NO: | 16) | |
| | | | | 15) | | |
| 7 | chr3 | 51729812 | 51729854 | TTTTTTGCCA | CCACCACCACCCC | No |
| | | | | ACACTCCT | ACCCT (SEQ ID NO: | |
| | | | | (SEQ ID NO: | 18) | |
| | | | | 17) | | |
| 8 | chr20 | 9514366 | 9514406 | TCCCCTCCAC | TCCCCTCAACCCA | No |
| | | | | CCCAGTTC | AAACT (SEQ ID NO: | |
| | | | | (SEQ ID NO: | 20) | |
| | | | | 19) | | |
| 9 | chr9 | 135125487 | 135125528 | TCCCCTCCTG | TCCCCTACCCCCC | No |
| | | | | CCCACAGA | AAACA (SEQ ID | |
| | | | | (SEQ ID NO: | NO: 22) | |
| | | | | 21) | | |
| 10 | chr17 | 16923065 | 16923106 | TTCTCACCAC | TCCCTTCCCACCC | No |
| | | | | CCCACACT | AAAGT (SEQ ID NO: | |
| | | | | (SEQ ID NO: | 24) | |
| | | | | 23) | | |
| 11 | chr5 | 177973159 | 177973194 | TCCCCTCCAC | CCCCCGCCACCCC | No |
| | | | | CTCAAACA | ATATT (SEQ ID NO: | |
| | | | | (SEQ ID NO: | 26) | |
| | | | | 25) | | |
| 12 | chr20 | 30459310 | 30459347 | TCACCCCCAC | CTCTCTATCCACA | No |
| | | | | CCCTCAAT | ATCCC (SEQ ID NO: | |
| | | | | (SEQ ID NO: | 28) | |
| | | | | 27) | | |
| 13 | chr19 | 1854107 | 1854149 | TCCACTCCAC | TCCTCTCCACCAC | No |
| | | | | CCCCCCAA | CCCCT (SEQ ID NO: | |
| | | | | (SEQ ID NO: | 30) | |
| | | | | 29) | | |
| 14 | chr22 | 51139503 | 51139538 | TCCCCCCACC | TTTTCTGTCCCCAC | No |
| | | | | CCCCAAAA | TCCA (SEQ ID NO: | |
| | | | | (SEQ ID NO: | 32) | |
| | | | | 31) | | |
| 15 | chr22 | 51139504 | 51139538 | CCCCCCACCC | TTTTCTGTCCCCAC | No |
| | | | | CCCAAAAA | TCCA (SEQ ID NO: | |
| | | | | (SEQ ID NO: | 34) | |
| | | | | 33) | | |
| 16 | chrX | 31479737 | 31479778 | TCTTCCATCA | TACCCTCCACCCT | No |
| | | | | CTAATTCT | ACCAT (SEQ ID NO: | |
| | | | | (SEQ ID NO: | 36) | |
| | | | | 35) | | |
| 17 | chr8 | 66106608 | 66106646 | TTTTCTGTAA | TGTTCTGACACTTT | No |
| | | | | CCACTCCT | TCCC (SEQ ID NO: | |
| | | | | (SEQ ID NO: | 38) | |
| | | | | 37) | | |
| 18 | chr10 | 71712424 | 71712459 | CCCCCTCCCA | TCCTCCCCACCCC | No |
| | | | | CCCACCTT | CCAGG (SEQ ID NO: | |
| | | | | (SEQ ID NO: | 40) | |
| | | | | 39) | | |
| 19 | chr10 | 71712418 | 71712459 | TCCCCTCCCC | TCCTCCCCACCCC | No |
| | | | | CTCCCACC | CCAGG (SEQ ID NO: | |
| | | | | (SEQ ID NO: | 42) | |
| | | | | 41) | | |
| 20 | chr19 | 35716209 | 35716246 | TCCTCGCCAC | CCTCCTCCACCCC | No |
| | | | | CCCCCACG | ACTGT (SEQ ID NO: | |
| | | | | (SEQ ID NO: | 44) | |
| | | | | 43) | | |
| 21 | chr1 | 23205692 | 23205728 | CCACCTCCAC | TCTCATCCAACCC | No |
| | | | | CACACACA | ACAGG (SEQ ID | |
| | | | | (SEQ ID NO: | NO: 46) | |
| | | | | 45) | | |
| 22 | chr18 | 66577716 | 66577755 | TTTTATATCA | TTTTCTGTGATCAA | No |
| | | | | CCAACCCC | TTAT (SEQ ID NO: | |
| | | | | (SEQ ID NO: | 48) | |
| | | | | 47) | | |
| 23 | chr12 | 23734089 | 23734128 | TTCTCTCTCC | TTCTCTATTAAAA | No |
| | | | | CCACTCCT | CTCCT (SEQ ID NO: | |
| | | | | (SEQ ID NO: | 50) | |
| | | | | 49) | | |
| 24 | chr6 | 167286954 | 167286986 | CCGTCAGTCA | CCCACTCCACCCC | No |
| | | | | CCCCTCCT | ACTGT (SEQ ID NO: | |
| | | | | (SEQ ID NO: | 52) | |
| | | | | 51) | | |
| 25 | chr22 | 51139505 | 51139538 | CCCCCACCCC | TTTTCTGTCCCCAC | No |
| | | | | CCAAAAAA | TCCA (SEQ ID NO: | |
| | | | | (SEQ ID NO: | 54) | |
| | | | | 53) | | |
| 26 | chr3 | 151635330 | 151635367 | TGCCCCCCAA | TTTTCTTTCACCAA | No |
| | | | | CCCACCAA | TACC (SEQ ID NO: | |
| | | | | (SEQ ID NO: | 56) | |
| | | | | 55) | | |
| 27 | chr20 | 25440058 | 25440099 | ACACCTGCAC | TTTTCTGTCTCAAA | No |
| | | | | CCCACATT | CCAT (SEQ ID NO: | |
| | | | | (SEQ ID NO: | 58) | |
| | | | | 57) | | |
| 28 | chr6 | 148392411 | 148392443 | TCCCCTCATC | TCCCCAACATCAC | No |
| | | | | CCCACATC | ACACT (SEQ ID NO: | |
| | | | | (SEQ ID NO: | 60) | |
| | | | | 59) | | |
| 29 | chr16 | 89289717 | 89289757 | TTCTGTGGAA | TTTGCCGTCACCA | No |
| | | | | CCAATACT | ACCCT (SEQ ID NO: | |
| | | | | (SEQ ID NO: | 62) | |
| | | | | 61) | | |
| 30 | chr11 | 57225403 | 57225434 | CCCCCTCCCC | TTTGCTGTCCCCAC | No |
| | | | | CCAACCCT | CCCA (SEQ ID NO: | |
| | | | | (SEQ ID NO: | 64) | |
| | | | | 63) | | |
| 31 | chr1 | 109800575 | 109800608 | CCCACTCCCC | TCCCCACTCCCCC | No |
| | | | | CCCACCCC | ACAGC (SEQ ID NO: | |
| | | | | (SEQ ID NO: | 66) | |
| | | | | 65) | | |
| 32 | chr1 | 227647992 | 227648030 | TTTTCTATTA | TCCCCTCCTCACC | No |
| | | | | ACAAAAAT | ACTGT (SEQ ID NO: | |
| | | | | (SEQ ID NO: | 68) | |
| | | | | 67) | | |
| 33 | chr20 | 19431018 | 19431059 | TTTCATCTCC | CCTCCTCCTCCCCA | No |
| | | | | CCAGCCCT | CAGT (SEQ ID NO: | |
| | | | | (SEQ ID NO: | 70) | |
| | | | | 69) | | |
| 34 | chr8 | 145493633 | 145493673 | TTTCCTCTCC | TCCTCTCCTCCCCT | No |
| | | | | CTAATCCT | CCCT (SEQ ID NO: | |
| | | | | (SEQ ID NO: | 72) | |
| | | | | 71) | | |
| 35 | chr8 | 145324548 | 145324588 | TTTCCTCTCC | TCCTCTCCTCCCCT | No |
| | | | | CTAATCCT | CCCT (SEQ ID NO: | |
| | | | | (SEQ ID NO: | 74) | |
| | | | | 73) | | |
| 36 | chr1 | 240682937 | 240682971 | TTTTCTGAAA | CTTTTTATCCCCAG | No |
| | | | | CCAATCCT | TACA (SEQ ID NO: | |
| | | | | (SEQ ID NO: | 76) | |
| | | | | 75) | | |
| 37 | chr6 | 101328557 | 101328598 | ATTTCTTTCC | TTTTCTGTTACAAA | No |
| | | | | CCAAATCT | GCCT (SEQ ID NO: | |
| | | | | (SEQ ID NO: | 78) | |
| | | | | 77) | | |
| 38 | chr7 | 81302717 | 81302749 | TTTTCTTACA | TTTACTGTCACCA | No |
| | | | | GCAACACT | CTACT (SEQ ID NO: | |
| | | | | (SEQ ID NO: | 80) | |
| | | | | 79) | | |
| 39 | chr1 | 62776649 | 62776690 | TATTCTGTCA | TCCCCTCCCACCCT | No |
| | | | | TCACTCCT | TAAC (SEQ ID NO: | |
| | | | | (SEQ ID NO: | 82) | |
| | | | | 81) | | |
| 40 | chr3 | 124702671 | 124702707 | TCCCTTCCAC | TGTTCTGTACCCA | No |
| | | | | CTCACCAA | ACCCT (SEQ ID NO: | |
| | | | | (SEQ ID NO: | 84) | |
| | | | | 83) | | |
| 41 | chr16 | 9446225 | 9446264 | TTCTCTCCAC | ATTGCTTACACCA | No |
| | | | | ACCACAGT | CTCCT (SEQ ID NO: | |
| | | | | (SEQ ID NO: | 86) | |
| | | | | 85) | | |
| 42 | chr3 | 188629607 | 188629647 | TCCCCACCAC | TTTTGAATCAACA | No |
| | | | | CCCAAAGA | CTCCC (SEQ ID NO: | |
| | | | | (SEQ ID NO: | 88) | |
| | | | | 87) | | |
| 43 | chr16 | 85176136 | 85176173 | TCCCCTCTCC | CCCCCTCCACAGC | No |
| | | | | CCCACAGT | CTCAT (SEQ ID NO: | |
| | | | | (SEQ ID NO: | 90) | |
| | | | | 89) | | |
| 44 | chr1 | 23443971 | 23444002 | CCCCCCCCCC | CCCCCCCCGCCCC | No |
| | | | | CCCACAAT | CCCCC (SEQ ID NO: | |
| | | | | (SEQ ID NO: | 92) | |
| | | | | 91) | | |
| 45 | chr2 | 27902887 | 27902923 | TCCCCCCAAC | CCCCTCCCACCCC | No |
| | | | | CCTTCATT | CCAGT (SEQ ID NO: | |
| | | | | (SEQ ID NO: | 94) | |
| | | | | 93) | | |
| 46 | chr3 | 1948778 | 1948810 | TTCTCTGTCA | TTTTACCTTAACA | No |
| | | | | CCAGCCCA | ATCCT (SEQ ID NO: | |
| | | | | (SEQ ID NO: | 96) | |
| | | | | 95) | | |
| 47 | chr5 | 165831361 | 165831403 | TTTTCTATAA | CCCCCTCCACCCC | No |
| | | | | CTCATATT | AAAAA (SEQ ID | |
| | | | | (SEQ ID NO: | NO: 98) | |
| | | | | 97) | | |
| 48 | chr4 | 182910724 | 182910754 | TTTCTTTTCAC | TTTTACATCAACA | No |
| | | | | TATTCCT (SEQ | ATCCT (SEQ ID NO: | |
| | | | | ID NO: 99) | 100) | |
| 49 | chr13 | 103511981 | 103512019 | TTTTCTTTCTG | TCCACACCACCCT | No |
| | | | | CAACCAT | ACAGT (SEQ ID NO: | |
| | | | | (SEQ ID NO: | 102) | |
| | | | | 101) | | |
| 50 | chr16 | 11588172 | 11588209 | CCCCCTTCAC | CCCCCCCCACACC | No |
| | | | | CCCACCCC | ACTGC (SEQ ID NO: | |
| | | | | (SEQ ID NO: | 104) | |
| | | | | 103) | | |
| 51 | chr1 | 210512105 | 210512143 | TATGCTGTCT | TCTTCTGCCACCA | No |
| | | | | CCATGCCT | GTCCT (SEQ ID NO: | |
| | | | | (SEQ ID NO: | 106) | |
| | | | | 105) | | |
| 52 | chr2 | 26398304 | 26398335 | ACCCCTCCAA | CCCCCTCATCCTCC | No |
| | | | | CCCTCAGC | CAGT (SEQ ID NO: | |
| | | | | (SEQ ID NO: | 108) | |
| | | | | 107) | | |
| 53 | chr5 | 127879161 | 127879199 | CCACCTCCAC | TCTTCTGACACCA | No |
| | | | | CTAACAGT | TCCCA (SEQ ID NO: | |
| | | | | (SEQ ID NO: | 110) | |
| | | | | 109) | | |
| 54 | chr15 | 66978743 | 66978773 | TCCACTCCAC | CCCCCTCAACCAC | No |
| | | | | CCCCTCTT | CCAGC (SEQ ID NO: | |
| | | | | (SEQ ID NO: | 112) | |
| | | | | 111) | | |
| 55 | chr2 | 172495090 | 172495127 | TCCCATCTCA | TTTTCCATCACCA | No |
| | | | | CCCAAAAT | ATTCA (SEQ ID NO: | |
| | | | | (SEQ ID NO: | 114) | |
| | | | | 113) | | |
| 56 | chr8 | 100340500 | 100340539 | TTGTCTTCCA | CTTTCTATCAGCA | No |
| | | | | CAAAACCT | CTCCT (SEQ ID NO: | |
| | | | | (SEQ ID NO: | 116) | |
| | | | | 115) | | |
| 57 | chr2 | 157188240 | 157188270 | TCCCCTCCCC | TCCCCCCACCCCC | No |
| | | | | CACAATCT | ACCAC (SEQ ID NO: | |
| | | | | (SEQ ID NO: | 118) | |
| | | | | 117) | | |
| 58 | chr8 | 126808188 | 126808223 | TTCTCTGTGC | TCTTCTGTCACCTG | No |
| | | | | ACAAGCCT | TCCT (SEQ ID NO: | |
| | | | | (SEQ ID NO: | 120) | |
| | | | | 119) | | |
| 59 | chr8 | 107823093 | 107823123 | TCCCCACCCT | CTCTCTGGCACCC | No |
| | | | | CCCACATT | TTCCT (SEQ ID NO: | |
| | | | | (SEQ ID NO: | 122) | |
| | | | | 121) | | |
| 60 | chr9 | 135721616 | 135721651 | TATCCAGACA | CCCCATCCACCCC | No |
| | | | | CCCACCCT | ACACA (SEQ ID NO: | |
| | | | | (SEQ ID NO: | 124) | |
| | | | | 123) | | |
| 61 | chr11 | 26039493 | 26039534 | ACCTCTGCCT | CCCCCTCCACCCC | No |
| | | | | CCCATCCT | CCGAT (SEQ ID NO: | |
| | | | | (SEQ ID NO: | 126) | |
| | | | | 125) | | |
| 62 | chr1 | 23751270 | 23751301 | CCACCCCCAC | ACCCCTCCCCCGC | No |
| | | | | CCCCCCAC | ACAGT (SEQ ID NO: | |
| | | | | (SEQ ID NO: | 128) | |
| | | | | 127) | | |
| 63 | chr5 | 152496951 | 152496988 | ATTTCTTTCC | TTCCCTGCCACCA | No |
| | | | | CCCATACT | ACCCC (SEQ ID NO: | |
| | | | | (SEQ ID NO: | 130) | |
| | | | | 129) | | |
| 64 | chr5 | 59268073 | 59268112 | CTTTCTCTCA | TACTCAGTATCCA | No |
| | | | | CCTATTCT | ATCCT (SEQ ID NO: | |
| | | | | (SEQ ID NO: | 132) | |
| | | | | 131) | | |
| 65 | chr12 | 127662432 | 127662471 | TCTCACCCAC | TGCCCTCCTACCC | No |
| | | | | CCAACAGT | ACAAC (SEQ ID NO: | |
| | | | | (SEQ ID NO: | 134) | |
| | | | | 133) | | |
| 66 | chr7 | 133549428 | 133549458 | TCTTCTGTCA | CTTTCTGGCACAT | No |
| | | | | CTAAAACT | ATACT (SEQ ID NO: | |
| | | | | (SEQ ID NO: | 136) | |
| | | | | 135) | | |
| 67 | chr3 | 151635331 | 151635367 | GCCCCCCAAC | TTTTCTTTCACCAA | No |
| | | | | CCACCAAA | TACC (SEQ ID NO: | |
| | | | | (SEQ ID NO: | 138) | |
| | | | | 137) | | |
| 68 | chr11 | 22605631 | 22605667 | TCCCCCCCAC | CCACCTCCACCAC | No |
| | | | | CCCGCCAA | AAGGT (SEQ ID NO: | |
| | | | | (SEQ ID NO: | 140) | |
| | | | | 139) | | |
| 69 | chr5 | 59756990 | 59757024 | TTTCCCTTCA | CTTTCTGTCACCCA | No |
| | | | | AACATCCA | TCAT (SEQ ID NO: | |
| | | | | (SEQ ID NO: | 142 | |
| | | | | 141) | | |
| 70 | chr19 | 38858131 | 38858173 | CCCCTCCAAC | TCTCCTGTCACCA | No |
| | | | | CCCATAGT | AATCT (SEQ ID NO: | |
| | | | | (SEQ ID NO: | 144) | |
| | | | | 143) | | |
| 71 | chr9 | 114125386 | 114125428 | TCCCCCCCAC | TTTCTTCTCCCCTA | No |
| | | | | CCCACCCA | ACCT (SEQ ID NO: | |
| | | | | (SEQ ID NO: | 146) | |
| | | | | 145) | | |
| 72 | chr21 | 36774097 | 36774136 | TCCTTTCCCA | TTCCTTCCACCCCA | No |
| | | | | CCAATCCT | CTGC (SEQ ID NO: | |
| | | | | (SEQ ID NO: | 148) | |
| | | | | 147) | | |
| 73 | chr6 | 75057747 | 75057788 | TTTTCTGTCC | TCCTCCCCACCAC | No |
| | | | | CCAAACAG | CAAAT (SEQ ID NO: | |
| | | | | (SEQ ID NO: | 150) | |
| | | | | 149) | | |
| 74 | chr14 | 100472345 | 100472376 | TCCCCCCCCC | TCACCCCCAACAC | No |
| | | | | CCCCCACA | ACACA (SEQ ID NO: | |
| | | | | (SEQ ID NO: | 152) | |
| | | | | 151) | | |
| 75 | chr12 | 125339766 | 125339804 | CCCCCTCCCC | TTCCCTCCACTCCA | No |
| | | | | AGCACAGG | CTGT (SEQ ID NO: | |
| | | | | (SEQ ID NO: | 154) | |
| | | | | 153) | | |
| 76 | chr16 | 89368527 | 89368566 | CTCCCTCCAC | TCCCCTCCTTCCCC | No |
| | | | | CCCACTCA | CAGA (SEQ ID NO: | |
| | | | | (SEQ ID NO: | 156 | |
| | | | | 155) | | |
| 77 | chr1 | 202164560 | 202164600 | CCCCCTCCAC | TTTTCTCTTCCCAC | No |
| | | | | TCCAAAAG | TCCC (SEQ ID NO: | |
| | | | | (SEQ ID NO: | 158) | |
| | | | | 157) | | |
| 78 | chrX | 151141431 | 151141469 | TACCCCCTCC | GCCCCTCCCCCCC | No |
| | | | | CAAACAGT | CCACT (SEQ ID NO: | |
| | | | | (SEQ ID NO: | 160) | |
| | | | | 159) | | |
| 79 | chr12 | 50475768 | 50475800 | TCTGCTGCCA | TCCCTTCCACCCCT | No |
| | | | | CCAGTCAC | CACT (SEQ ID NO: | |
| | | | | (SEQ ID NO: | 162) | |
| | | | | 161) | | |
| 80 | chr14 | 57361405 | 57361444 | TCCCCTCCAA | CTTTCATTCACAAT | No |
| | | | | CCCAAACC | CCCT (SEQ ID NO: | |
| | | | | (SEQ ID NO: | 164) | |
| | | | | 163) | | |
| 81 | chr7 | 94322229 | 94322265 | TCTTATTTCA | TTTCCTCTCACCAG | No |
| | | | | CTAAGACT | TCCT (SEQ ID NO: | |
| | | | | (SEQ ID NO: | 166) | |
| | | | | 165) | | |
| 82 | chr18 | 45316703 | 45316741 | AGCCCTCCAA | TCTTTTCCTCCCCA | No |
| | | | | CCCACAGT | CAAT (SEQ ID NO: | |
| | | | | (SEQ ID NO: | 168) | |
| | | | | 167) | | |
| 83 | chr5 | 134897098 | 134897136 | TCCCTCCCAC | ACTTCCCCACCCC | No |
| | | | | CACACAGC | ACAGC (SEQ ID NO: | |
| | | | | (SEQ ID NO: | 170) | |
| | | | | 169) | | |
| 84 | chr13 | 22412475 | 22412508 | TCACCTCCCC | TCTTCACTCCCCA | No |
| | | | | CCAAAAAT | AAACT (SEQ ID NO: | |
| | | | | (SEQ ID NO: | 172) | |
| | | | | 171) | | |
| 85 | chr7 | 106691643 | 106691681 | TTTTCTGTCTT | TTTTCTTTAGCCCA | No |
| | | | | CCATCAA | TCCT (SEQ ID NO: | |
| | | | | (SEQ ID NO: | 174) | |
| | | | | 173) | | |
| 86 | chr9 | 9197758 | 9197799 | TTTCCTGTCA | CATTCCTTCAAAA | No |
| | | | | ACACTCCT | ATCAT (SEQ ID NO: | |
| | | | | (SEQ ID NO: | 176) | |
| | | | | 175) | | |
| 87 | chr18 | 73098165 | 73098199 | TTTTCTCTCCT | TTTTCTGGCATCAT | No |
| | | | | CTCTCCT (SEQ | TCAT (SEQ ID NO: | |
| | | | | ID NO: 177) | 178) | |
| 88 | chr1 | 19736944 | 19736978 | TCCCCCCAAA | CCCCCACCACCCC | No |
| | | | | CACACACA | CCACC (SEQ ID NO: | |
| | | | | (SEQ ID NO: | 180) | |
| | | | | 179) | | |
| 89 | chr12 | 54204713 | 54204755 | TTCCCTCTCT | CTTTCTCTCCCCAA | No |
| | | | | CTACTCCT | TTCT (SEQ ID NO: | |
| | | | | (SEQ ID NO: | 182) | |
| | | | | 181) | | |
| 90 | chr16 | 74524192 | 74524225 | CTTTATCTCC | TTTTCTATCACTAA | No |
| | | | | CCAAGCAA | ACCT (SEQ ID NO: | |
| | | | | (SEQ ID NO: | 184) | |
| | | | | 183) | | |
| 91 | chr4 | 56586353 | 56586394 | GTTTATGTCC | ATTTATATCCACA | No |
| | | | | CCAATCCC | ATCAT (SEQ ID NO: | |
| | | | | (SEQ ID NO: | 186) | |
| | | | | 185) | | |
| 92 | chr19 | 35716204 | 35716246 | CCCCCTCCTC | CCTCCTCCACCCC | No |
| | | | | GCCACCCC | ACTGT (SEQ ID NO: | |
| | | | | (SEQ ID NO: | 188) | |
| | | | | 187) | | |
| 93 | chr15 | 46816195 | 46816234 | TGTTCTGTAA | TTTTCTTGCCCAGA | No |
| | | | | CCAATACT | CCCT (SEQ ID NO: | |
| | | | | (SEQ ID NO: | 190) | |
| | | | | 189) | | |
| 94 | chr3 | 49903243 | 49903280 | CACCCCCCAC | TCCCACCCACGCC | No |
| | | | | CCCACTCC | ACACT (SEQ ID NO: | |
| | | | | (SEQ ID NO: | 192) | |
| | | | | 191) | | |
| 95 | chr21 | 39941092 | 39941133 | TCCCCCCCCA | TATTCATTCAACA | No |
| | | | | CCCCCAGT | AACAT (SEQ ID NO: | |
| | | | | (SEQ ID NO: | 194) | |
| | | | | 193) | | |
| 96 | chr10 | 113458996 | 113459030 | TTTTCCCTTA | TTTTCCCTCACCAA | No |
| | | | | CCAATCTA | TCTA (SEQ ID NO: | |
| | | | | (SEQ ID NO: | 196) | |
| | | | | 195) | | |
| 97 | chr20 | 30586833 | 30586874 | CCCCCACCCC | TCCTCTCCCACCA | No |
| | | | | CCCACACA | ATCAC (SEQ ID NO: | |
| | | | | (SEQ ID NO: | 198) | |
| | | | | 197) | | |
| 98 | chr1 | 23443971 | 23444005 | CCCCCCCCCC | TCCCCCCCCCCGC | No |
| | | | | CCCACAAT | CCCCC (SEQ ID NO: | |
| | | | | (SEQ ID NO: | 200) | |
| | | | | 199) | | |
| 99 | chr4 | 75724475 | 75724505 | TTCCCTCCTT | TTTCCTGTCACCAT | No |
| | | | | CCCCCAAC | TTCT (SEQ ID NO: | |
| | | | | (SEQ ID NO: | 202) | |
| | | | | 201) | | |

Differentiation of ASPA-CD68 iPSCs into iOPCs: The ASPA-CD68 iPSCs were differentiated into iOPCs by following a previously published protocol [10c, 10d]. Briefly, the ASPA-CD68 iPSCs were dissociated into single cells and induced by OPC-I Medium containing DMEM/F12, 1×N2, 2 mM GlutaMAX, 0.1 μM RA (Sigma, R2625), 10 μM SB431542 (Peprocell, 04-0010-10), and 250 nM LDN-193189 (Peprocell, 04-0074-10) for 8 days. Then the cells were switched to OPC-II Medium containing DMEM/F12, 1×N2, 2 mM GlutaMAX, 0.1 μM RA and 1 μM SAG (Sigma, ML1314) for another 4 days. After 12 days of culture, the cells were dissociated and cultured in flasks for overnight to form spheres. The resultant pre-OPC spheres were switched to OPC-III Medium containing DMEM/F12, 1×N2, 1×B27 minus vitamin A (Thermo Fisher, 12587010), 2 mM GlutaMAX, 0.1 μM RA and 1 μM SAG for 8 days, and then switched to PDGF medium containing DMEM/F12, 1×N2, 1×B27 minus vitamin A, 2 mM GlutaMAX, 10 ng/ml PDGF-AA (R&D, 221-AA-050), 10 ng/ml IGF-1 (R&D, 291-GG-01M), 5 ng/ml HGF (R&D, 294-HG-250), 10 ng/ml NT3 (EMD Millipore, GF031; and PeproTech, AF-450-03), 60 ng/ml T3 (Sigma, T2877), 100 ng/ml Biotin (Sigma, 4639), 1 μM cAMP (Sigma, D0627), and 25 μg/ml Insulin (Sigma, 19278) for 10 days. After 18 days of suspension culture, the spheres were attached on Matrigel-coated plates and cultured for 30 to 60 days in the PDGF medium. The OPCs could be detected by flow cytometry with a CD140a antibody and by live staining with an O4 antibody after 30 days of attached culture. After 30 to 60 days of attached culture, the OPCs were collected for transplantation.

Flow cytometry: The human H9 ESCs (WiCell, WA09) were used as the positive control for FACS analysis to detect the pluripotency marker OCT4 and the human ESC cell surface marker SSEA4. The HEK293T cells were used as the negative cell control for iPSC and NPC marker detection. The cells were dissociated and passed through a 70 μm cell strainer to make single cell suspension. For cell surface marker staining, the cells were directly incubated with the fluorophore-conjugated primary antibodies for 20 minutes on ice. The same fluorophore-conjugated IgGs were included as the isotype controls. For intracellular OCT4 staining, the cells were first fixed and permeabilized using a Fixation/Permeabilization Solution Kit (BD, 554714) before incubation with the PE-conjugated anti-Oct3/4 primary antibody. The PE-conjugated mouse IgG1 was included as the isotype control. The cells were washed twice and resuspended in PBS containing DAPI and 0.1% donkey serum. The samples were run on Attune NxT Flow Cytometer (ThermoFisher Scientific) and the data were analyzed by FlowJo v10. The detailed information of all the primary antibodies and isotype controls used were listed in Table 3.

TABLE 3
List of Antibodies

| Antibody Name | Vendor | CAT. # |
|---|---|---|
| Rabbit monoclonal anti-NANOG | Cell Signaling | Cat# 4903 |
| Mouse monoclonal anti-OCT4 | Santa Cruz | Cat# sc-5279 |
| Goat polyclonal anti-SOX2 | Santa Cruz | Cat# sc-17320 |
| Mouse monoclonal anti-SSEA4 | Santa Cruz | Cat# sc-21704 |
| Mouse monoclonal IgM anti-Tra-1-60 Santa | Santa Cruz | Cat# sc-21705 |
| Cruz | | |
| Mouse monoclonal IgM anti-Tra-1-81 Santa | Santa Cruz | Cat# sc-21706 |
| Cruz | | |
| Mouse monoclonal anti-NESTIN | Fisher (BD | Cat# 611659 |
| | Biosciences) | |
| Goat polyclonal anti-SOX1 | R&D | Cat# AF3369 |
| PE-conjugated anti-SSEA4 | BD Biosciences | Cat# 560128 |
| PE-conjugated mouse IgG3, isotype control | BD Biosciences | Cat# 559926 |
| (SSEA4 control) | | |
| APC-conjugated anti-CD133 | BD Biosciences | Cat# 566596 |
| APC-conjugated mouse IgG1, isotype control | BD Biosciences | Cat# 554681 |
| (CD133 control) | | |
| PE-conjugated anti-Oct3/4 | BD Biosciences | Cat# 560186 |
| PE-conjugated mouse IgG1, isotype control | BD Biosciences | Cat# 554680 |
| (Oct3/4 control) | | |
| Mouse monoclonal IgM anti-O4 | Sigma-Aldrich | Cat# O7139 |
| PE-conjugated anti-CD140a | BD Biosciences | Cat# 556002 |
| PE-conjugated mouse IgG2a, isotype control | BD Biosciences | Cat# 555574 |
| (CD140a control) | | |
| APC-conjugated anti-CD19 | ThermoFisher | Cat# |
| | Scientific | MHCD1905 |
| Mouse monoclonal anti-human nuclear | Abcam | Cat# |
| antigen antibody [235-1], hNu | | Ab191181 |
| Rabbit polyclonal anti-PAX6 | Biolegend | Cat# 901301 |
| Rabbit polyclonal anti-OLIG2 | Millipore | Cat# AB9610 |
| Rabbit polyclonal anti-GFAP | Agilent (Dako) | Cat# Z033429-2 |
| Goat polyclonal anti-SOX9 | R&D | Cat# AF3075 |
| Goat polyclonal anti-SOX10 | R&D | Cat# AF2864 |
| Rabbit polyclonal anti-NEUN | GeneTex | Cat# |
| | | GTX16208 |
| Rabbit monoclonal anti-Ki67 | ThermoFisher | Cat# RM-9106-S0 |
| | Scientific | |

Immunocytochemistry: The cells were fixed with 4% PFA at room temperature (RI) for 10 minutes. After fixation, the cells were washed with PBS twice and blocked with 5% donkey serum diluted in PBS with 0.1% triton (PBST) for 1 hour at RT. The fixed cells were then incubated with primary antibodies at 4° C. for overnight. On the following day, the cells were washed with PBS twice, incubated with the secondary antibodies at RT for 1 hour and washed. The cells were counterstained with DAPI before mounting for imaging. The images were taken using Nikon ECLIPSE TE2000-S or Nikon Ti-2. The detailed information of the primary antibodies used was listed in Table 3.

Viability assay: The vials with the frozen cells were thawed in a 37° C. water bath and the content was transferred to a 15 mL conical tube. Three mL medium was added drop by drop and the cell suspension was centrifuged at 200×g for 3 minutes. The cell pellet was resuspended in Perfusion Fluid CNS (CMAP000151, Harvard Apparatus). A small aliquot of cell suspension was further diluted by Trypan blue solution. The live and dead cells were counted by Hemocytometer. Three cryopreserved vials were tested for each cell lines.

Sterility and endotoxin test: One to two mL media were collected from culturing plates or flasks and sent to Department of Pathology in City of Hope to test for sterility. One mL media were collected from culturing plates or flasks and sent to Center for Biomedicine and Genetics and Analytical Pharmacology Core Facility of City of Hope to test for endotoxin.

Karyotype and Short Tandem Repeat (STR) analysis: The iPSCs in culture were directly sent to the Cyotogenetics Core of City of Hope for karyotype analysis using standard G-banding method. Total 20 metaphase cells were analyzed for each sample. For STR assay, the DNA was first purified from the fibroblasts, iPSCs and ASPA iNPCs. Geneprint 10 System PCR Amplification Kit (Promega, B9510) was used to generate a 10-locus DNA profile that is unique to each individual. PCR products were sent to City of Hope Integrative Genomics Core for fragment analysis. The results were analyzed using the GeneMapper™ Software 5 (Thermo Fisher).

Exon sequencing of the ASPA genomic DNA: The genomic DNAs were extracted from CD iPSCs using QuickExtract™ DNA Extraction Solution (Lucigen, QE09050. The primers used for sequencing each exon were listed in Table 4.

TABLE 4
List of Primers

| Primer Name | Sequence |
|---|---|
| ASPA-Exon1-Fwd | 5′-CTCCACTCAAGGGAATTCTGT-3′ (SEQ ID NO: 203) |
| ASPA-Exon1-Rev | 5′-ACTGCATGTACGGACATGAA-3′ (SEQ ID NO: 204) |
| ASPA-Exon2-Fwd | 5′-AGATTTGGCGACTGGTTCT-3′ (SEQ ID NO: 205) |
| ASPA-Exon2-Rev | 5′-TGCACCTTCCCTCATAACTG-3′ (SEQ ID NO: 206) |
| ASPA-Exon3-Fwd | 5′-ACTCTGTTGAAGCAAAGAGA-3′ (SEQ ID NO: 207) |
| ASPA-Exon3-Rev | 5′-CAGAGCAAGACTCTGTCTCA-3′ (SEQ ID NO: 208) |
| ASPA-Exon4-Fwd | 5′-TTCCATGATGCTACATGGTT-3′ (SEQ ID NO: 209) |
| ASPA-Exon4-Rev | 5′-GCAAATCTGACCCAGGTTCCA-3′ (SEQ ID NO: 210) |
| ASPA-Exon5-Fwd | 5′-TGTTCTCGAACTCCTGACCT-3′ (SEQ ID NO: 211) |
| ASPA-Exon5-Rev | 5′-GCGAAGTGCTGTATGAGCTA-3′ (SEQ ID NO: 212) |
| ASPA-Exon6-Fwd | 5′-GATCAAGACTGGAAACCAC-3′ (SEQ ID NO: 213) |
| ASPA-Exon6-Rev | 5′-GAAGTGTAGTAAGGCAAAGC-3′ (SEQ ID NO: 214) |
| Endo-OCT4-Fwd | 5′-CCTCACTTCACTGCACTGTA-3′ (SEQ ID NO: 215) |
| Endo-OCT4-Rev | 5′-CAGGTTTTCTTTCCCTAGCT-3′ (SEQ ID NO: 216) |
| Endo-SOX2-Fwd | 5′-CCCAGCAGACTTCACATGT-3′ (SEQ ID NO: 217) |
| Endo-SOX2-Rev | 5′-CCTCCCATTTCCCTCGTTTT-3′ (SEQ ID NO: 218) |
| Endo-NANOG-Fwd | 5′-GAATCTTCACCTATGCCTGTG-3′ (SEQ ID NO: 219) |
| Endo-NANOG-Rev | 5′-ATCATTGAGTACACACAGCTG-3′ (SEQ ID NO: 220) |
| Exo-OCT4-Fwd | 5′-CTCTAGAGCCTCTGCTAACCA-3′ (SEQ ID NO: 221) |
| Exo-OCT4-Rev | 5′-TGTGCATAGTCGCTGCTTGAT-3′ (SEQ ID NO: 222) |
| Exo-KLF4-Fwd | 5′-GCTCCCATCTTTCTCCACGTT-3′ (SEQ ID NO: 223) |
| Exo-KLF4-Rev | 5′-GAAGCTTGAATTCCTGCAGGCA-3′ (SEQ ID NO: 224) |
| Exo-LIN28-Fwd | 5′-AGAGCATCAGCCATATGGTAG-3′ (SEQ ID NO: 225) |
| Exo-LIN28-Rev | 5′-GAAGCTTGAATTCCTGCAGGCA-3′ (SEQ ID NO: 226) |
| Exo-L-MYC-Fwd | 5′-CTCTAGAGCCTCTGCTAACCA-3′ (SEQ ID NO: 227) |
| Exo-L-MYC-Rev | 5′-TCGAATTTCTTCCAGATGTCC-3′ (SEQ ID NO: 228) |
| ASPA-Fwd | 5′-CGGAATTCATGACTTCTTGTCAC-3′ (SEQ ID NO: 229) |
| ASPA--Rev | 5′-GGACTAGTCTAATGTAAACAGCAG-3′ (SEQ ID NO: 230) |
| ACTIN-Fwd | 5′-CCGCAAAGACCTGTACGCCAAC-3′ (SEQ ID NO: 231) |
| ACTIN-Rev | 5′-CCAGGGCAGTGATCTCCTTCTG-3′ (SEQ ID NO: 232) |
| SOX1-Fwd | 5′-AATACTGGAGACGAACGCCG-3′ (SEQ ID NO: 233) |
| SOX1-Rev | 5′-AGTGCTTGGACCTGCCTTAC-3′ (SEQ ID NO: 234) |
| PAX6-Fwd | 5′-GTGTCCAACGGATGTGTGAG-3′ (SEQ ID NO: 235) |
| PAX6-Rev | 5′-CTAGCCAGGTTGCGAAGAAC-3′ (SEQ ID NO: 236) |
| AAVS1-Fwd | 5′-CTCTAACGCTGCCGTCTCTC-3′ (SEQ ID NO: 237) |
| AAVS1-Rev | 5′-GCTTCTCCTCTTGGGAAGTG-3′ (SEQ ID NO: 238) |
| ASPA-Rev | 5′-AGCTCATTCCCATGGGTTCC-3′ (SEQ ID NO: 239) |
| PBS/psi-Fwd | 5′-ACTTGAAAGCGAAAGGGAAAC-3′ (SEQ ID NO: 240) |
| PBS/psi-Rev | 5′-TTTGGCGTACTCACCAGTC-3′ (SEQ ID NO: 241) |
| PBS/psi-TaqMan | 5′-FAM-AGCTCTCTCGACGCAGGACTCGGC-TAMRA-3′ |
| probe | (SEQ ID NO: 242) |
| Albumin-Fwd | 5′-TGAAACATACGTTCCCAAAGAGTTT-3′ (SEQ ID NO: 243) |
| Albumin-Rev | 5′-CTCTCCTTCTCAGAAAGTGTGCATAT-3′ (SEQ ID NO: 244) |
| Albumin-TaqMan | 5′-FAM-TGCTGAAACATTCACCTTCCATGCAGA-TAMRA-3′ |
| probe | (SEQ ID NO: 245) |
| PBS/psi- | 5′- |
| gBlock | TCTAGCAGTGGCGCCCGAACAGGGACTTGAAAGCGAAAGGGAAACCA |
| | GAGGAGCTCTCTCGACGCAGGACTCGGCTTGCTGAAGCGCGCACGGC |
| | AAGAGGCGAGGGGCGGCGACTGGTGAGTACGCCAAAAATTTTGACTA |
| | GCGGAGGCT-3′ (SEQ ID NO: 246) |
| Albumin-gBlock | 5′- |
| | CATGGCGGCCGCGGGAATTCGATTTGAAACATACGTTCCCAAAGAGTT |
| | TAATGCTGAAACATTCACCTTCCATGCAGATATATGCACACTTTCTGA |
| | GAAGGAGAGAATCACTAGTGAATTCGCGG-3′ (SEQ ID NO: 247) |
| REX1 | Hs01938187_s1 (Thermo Fisher Cat# 4331182) |

ASPA enzymatic activity assay for ASPA iNPCs: The ASPA enzymatic assay was developed in the laboratory based on a published protocol [16, 45]. The cell lysates were prepared using RIPA buffer with PMSF and the protein concentration was determined by Bradford. For the first reaction, 100 μg protein lysates in 50 μL RIPA buffer was mixed with 50 μL 2×Assay Buffer I with the final concentration of 50 mM Tris-HCl, pH8.0, 50 mM NaCl, 0.1 mM DTT, 0.05% IGEPAL CA-630, 2.5 mM CaCl₂), and 5 mM NAA. The reaction mixture was incubated at 37° C. for 1 hour, and the reaction was stopped by heating the tubes at 100° C. for 3 minutes. After centrifugation at 15,000 g for 5 minutes, the supernatant was collected for the second reaction. For the second reaction, 90 μL of the first reaction supernatant was added to 90 μL 2×Assay Buffer II with the final concentration of 50 mM Tris-HCl pH 8.0, 50 mM NaCl, 2.5 mM alpha-ketoglutarate (AKG), 1 mg/mL BSA, 5 μM PLP, 0.5 mM p-NADH, 10 units MDH, and 10 unit glutamate-oxalacetate transaminase (GOT). Twenty minutes later, OD 340 nm was determined by luminescence reader. The ASPA activity is defined by the production of aspartate in nmol by 1 mg protein lysate in 1 hour at 37° C.

ASPA transgene copy number analysis: Because the human ASPA transgene in the lentiviral vector was integrated into the genome together with the PBS/psi region, the copy number of the human ASPA transgene was measured by detecting the PBS/psi region [46]. Specifically, the ASPA transgene copy number was detected by TaqMan real time PCR using Step One Plus real-time PCR system (Applied Biosystems) with primers in the PBS/psi region: PBS/psi-Fwd and PBS/psi-Fwd, and the PBS/psi-TaqMan probe. The Albumin gene is a single copy gene in the genome (2 copies/cell). It was included as an internal control and amplified using primers: Albumin-Fwd and Albumin-Rev, and the Albumin-TaqMan probe. The gBlock DNA fragment mixtures of psi and albumin with different ratio were amplified to create a standard curve to determine the relationship between ΔCt (psi-albumin) and log 2(psi copy number). If the log 2 (psi copy number) is n for the unknown sample, the transduced hASPA copy number/Cell=power (2, n). The Ct values were determined by TaqMan real time PCR, and used to calculate the copy numbers of both Albumin and the ASPA transgene based on the standard curves. The primers and gBlocks used were listed in Table 4.

RNA preparation and RT-PCR analysis: Total RNAs were extracted from cells using TRlazol (Invitrogen, 15596018). Reverse transcription was performed with 1 μg of RNA using the Tetro cDNA synthesis kit (Bioline, BIO-65043). Real-time PCR was performed using DyNAmo Flash SYBR Green qPCR mix on a StepOnePlus system (Applied Biosciences) and normalized to β-actin. The primers used for PCR are listed in Table 4.

Generation and maintenance of immunodeficient CD (Nur7) mice: All animal housing conditions and surgical procedures were approved by and conducted according to the Institutional Animal Care and Use Committee of City of Hope. The ASPA^{nur7/+} (ASPA^{nur7}/J, 008607) and Rag2^{-/-} mice (B6(Cg)-Rag^{2tm1, 1Cgn}/J, 008449) were purchased from the Jackson Laboratory. The ASPA^{nur7/+} mice were backcrossed with Rag2^{-/-} mice for four generations and screened for homozygosity of ASPA^{nur7/nur7} and Rag2^{-/-} mutations. The ASPA^{nur7/nur7}/Rag2^{-/-} mice were called CD (Nur7) mice. The survival of the WT, Het, and CD (Nur7) mice, and the ASPA iNPC-transplanted CD (Nur7) mice was monitored for 10 months. The animal death during the first 2 months for mice of all genotypes was not counted, because it was impossible to differentiate death resulted from pathology versus death resulted from events associated with fostering, cannibalization, and weaning occurred during this period.

Stereotaxic transplantation: The postnatal day 1 to 4 (PND 1-4) mice were anesthetized on ice for 6-7 minutes and then placed onto a stereotaxic device. The ASPA iNPCs in suspension were transplanted at 600,000 cells (in 1.5 μL) per site into six sites in the mouse brain bilaterally using a Hamilton syringe with a 33-gauge needle. The following coordinates, which were modified from a published study [47], were used for transplantation: the corpus callosum (+3.0, ±1.6, −1.3), the subcortical (0.5, ±1.0, −2.5), and the brain stem (−1.6, ±0.8, −3.0). For pups with weight over 2 g and/or with head size obviously bigger than usual, slightly modified coordinates were used: the corpus callosum (+3.5, ±1.7, −1.4), the subcortical (0.5, ±1.0, −2.5), and the brain stem (−1.6, ±1.0, −3.1). All the coordinates are (A, L, V) with reference to Lambda. “A” stands for anteroposterior from midline, “L” stands for lateral from midline, and “V” stands for ventral from the surface of brain, respectively. The ASPA iOPCs were transplanted with about 60,000 cells (in 1.5 μL) per site into six sites per mouse brain using the same coordinates.

Immunohistochemistry: Immunohistochemistry was performed on PFA-fixed tissues. Animals were deeply anesthetized and transcardially perfused with ice cold 0.9% saline followed by 4% PFA. The perfused brains were removed and post-fixed in 4% PFA, then cryoprotected with 30% sucrose. Cryoprotected brains were flash frozen and stored at −20° C. Then the brains were serially cryosectioned at sagittal planes. Specifically, slides were first labeled. Serial sections were collected onto labeled slides with one section per slide, until all slides were used for collection. The procedure was repeated until all sections from a brain were collected. For immunohistochemistry analysis, the brain sections were permeabilized in PBST for 2×10 minutes, blocked with 5% donkey serum in PBST for 1 hour at RT. Sections were then incubated with primary antibodies (Table 3) at 4° C. for overnight. Following primary antibody incubation and washes, sections were incubated with secondary antibodies at RT for 2 hours, washed with 1×PBS, counterstained with Dapi, and mounted with the mounting medium. Cell fate and proliferation status were assessed by double immunostaining using the anti-human nuclear antigen (hNA) together with antibodies against PAX6, NeuN, SOX9, OLIG2, or Ki67. Confocal microscopy was performed on a Zeiss LSM 700 microscope (Zeiss), and the resulting images were analyzed with Zen 2.3 lite software (Zeiss). For quantification, the images of transplanted cells in all three targeting sites including the corpus callosum, the subcortical and the brain stem regions were taken. Total human cells and double positive cells were counted for each brain. Three brains were analyzed in each group. The tiled whole section sagittal images were taken using Nikon Ti-2 and dot maps were made using Photoshop CS4 based on the hNu⁺ signal from the titled whole section sagittal images.

NAA level and ASPA activity measurement in brain tissues: The aqueous metabolites were extracted from mouse brains using the method of perchloric acid (PCA, Sigma, 244252) as described [48]. Briefly, the mouse brains were rapidly chopped into small pieces, mixed well and divided into aliquots. Two aliquots were placed into two 1.5 ml Eppendorf tubes. The brain tissues in one tube were subjected to PCA extraction directly, while tissues in another tube were incubated at 37° C. for 1 hour followed by PAC extraction. 6% ice-cold PCA was added into each tube at 5 ml per gram of the wet brain tissues, followed by vortexing for 30 seconds. The samples were incubated on ice for additional 10 minutes. The mixture was centrifuged at 12,000 g for 10 minutes at 4° C. The supernatant was transferred into a new tube, neutralized with 2 M K₂CO₃, and placed on ice with lids open to allow CO₂ to escape. Each sample was incubated on ice for 30 minutes to precipitate the potassium perchlorate salt. The supernatant was collected and the pH was adjusted to 7.4±0.2. The samples were centrifuged at 12,000 g for 10 minutes at 4° C. The supernatant was transferred to Eppendorf tubes and frozen on dry ice. The samples were then subjected to NMR analysis at the NMR Core Facility of City of Hope. The ASPA activity was calculated using the difference of NAA levels before and after 1 hour incubation at 37° C., and expressed as decreased NAA level in nmol per gram of brain tissue per hour.

H&E staining and vacuolation analysis: A one-in-six series of whole brain slides were stained with hematoxylin and eosin (H&E) at the Pathology Core of City of Hope. The whole slide was scanned under Nanozoomer HT (Hamamatsu Photonics, Japan) at the Light Microscopy Core of City of Hope. The surface area of the vacuolated brain regions and the intact brain regions was measured using Image-Pro Premier 9.2 for all sections. The percent vacuolation=[the area of vacuolated brain region/(the area of vacuolated brain region+the area of intact brain region)]×100. All sections from one representative slide of each brain were analyzed and at least three brains were analyzed for each mouse group.

Electron microscopy (EM) and G-ratio analysis of myelin sheaths: The mice were deeply anesthetized with isoflurane, and perfused with 0.9% saline followed by 0.1 M Millonig's buffer containing 4% paraformaldehyde (PFA) and 2.5% glutaraldehyde. The brain tissues were dissected and post-fixed in the same fixative overnight. A heavy metal staining protocol developed by Dr. Mark Ellisman's group [49] was followed. The target tissues were cut into ˜150 μm vibratome sections using a Leica VT 1000S vibratome. The subcortical white matter of the brain was micro-dissected and embedded in Durcupan ACM resin (Electron Microscopy Sciences). The ultra-thin sections were cut using a Leica Ultracut UCT ultramicrotome and picked onto EM grids. Transmission electron microscopy was performed on an FEI Tecnai 12 transmission electron microscope equipped with a Gatan Ultrascan 2K CCD camera at the EM Core Facility of City of Hope. Three to four images were randomly taken for each sample in the subcortical region (3 images for the HET and the transplanted mice, respectively, and 4 images for CD (Nur7) mice). The inner axonal diameter and the total outer diameter of total 15 myelin sheathes in the brain of the Het and the transplanted mice, respectively, and 17 myelin sheathes in the brain of the CD (Nur7) mice were measured using Image-Pro Premier 9.2. The g-ratio is the ratio of the inner axonal diameter to the total outer diameter. The abnormal myelin sheaths were further identified based on the layer structure of the myelin sheaths which exhibited substantial difference between the Het and the CD (Nur7) mice.

Rotarod test: The motor performance of the ASPA iNPC-transplanted mice was evaluated using a rotarod treadmill (Rotamex, Columbus Instruments) as described [17]. The mice were tested for the latency on the rod when the rod was rotating at the accelerating speed (2-65 rpm) in a 2-minute trial session. Each mouse was monitored for the latency 4 times per test. At least 6 mice for each group were tested.

Grip strength test: The forelimb strength of the transplanted mice was measured using a grip strength meter (BIO-GS3, Bioseb) to detect motor coordination and motor function. The mouse was allowed to grip a metal grid tightly. The grip strength of the mouse was recorded by gently pulling the tail of the mouse backward until release. Four sequential measurements were performed, and the average strength was calculated. At least 6 mice for each group were tested.

Mycoplasma test: All cell culture products including the iPSCs, iNPCs and iOPCs were checked for potential mycoplasma contamination using MycoAlert PLUS Mycoplasma Detection Kit (Lonza). Five hundred μL culture medium was harvested from each cell line and centrifuged at 200×g for 5 minutes to eliminate cell debris. One hundred μL medium was used for each reaction and duplicate reactions were run for each sample. The result was determined by luminescence reading according to the established SOP. All cellular products used in this study were mycoplasma negative.

Statistical analyses: The data are shown as means±SE as specified in the figure legends and analyzed with GraphPad Prism 8 (San Diego, CA) and KaleidaGraph 4.0 (Reading, PA). The number of mice analyzed per treatment group is indicated as “n” in the corresponding figure legends. No exclusion criteria were applied. The animals were assigned randomly to treatment groups. The study was not blinded. The student's t-test (two tailed), Log-rank test and One-Way ANOVA followed by Dunnett's multiple comparisons test or Tukey's multiple comparisons test were used for statistical analysis as reported in each figure legend. p<0.05 was considered statistically significant. *P<0.05, **P<0.01 and ***P<0.001.

### Example 2: Manufacturing Canavan Disease Patient iPSCs and Differentiating them into iNPCs

The example establishes human iPSC-based cell therapies for CD. It has been demonstrated that research-grade neural progenitor cells (NPCs) derived from CD patient iPSCs that were transduced with a wild type ASPA gene are able to ameliorate disease phenotypes in a CD (Nur7) mouse model in the developmental stage study. To move the therapeutic candidate to the clinic, Good Manufacturing Practice (GMP)-compatible processes were developed to manufacture the CD patient iPSC-derived cellular product. A GMP-compatible process was established to derive human iPSCs by episomal reprogramming [12] in an integration-free, xeno-free and feeder-free manner. Methods were further developed to expand human iPSCs and differentiate them into neural progenitor cells (iNPCs) under chemically defined, xeno-free and feeder-free, GMP-compatible conditions.

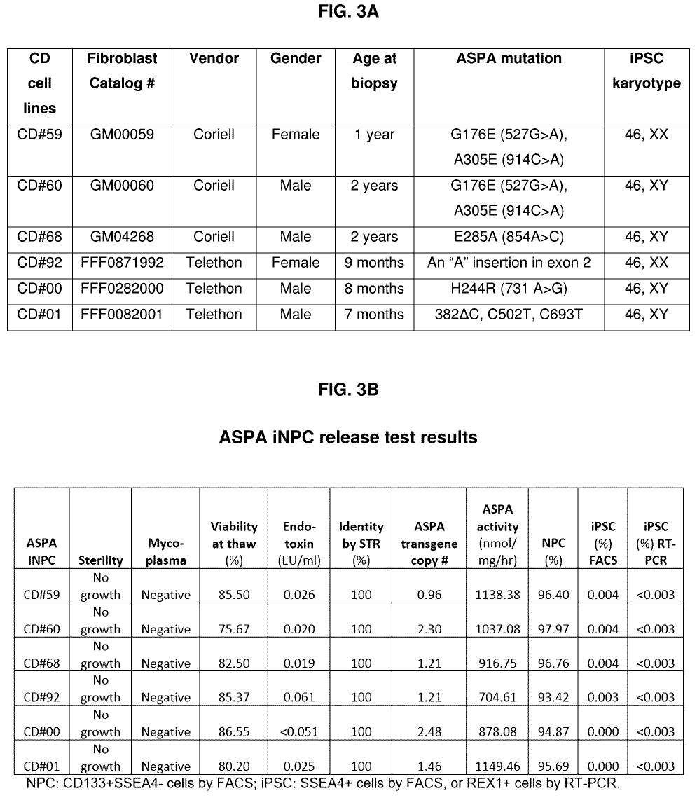

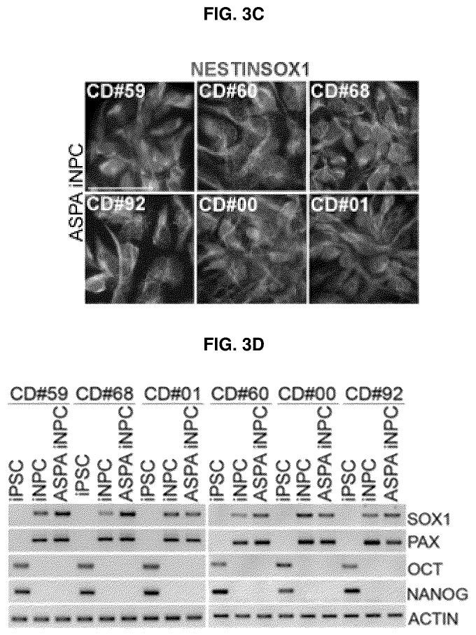

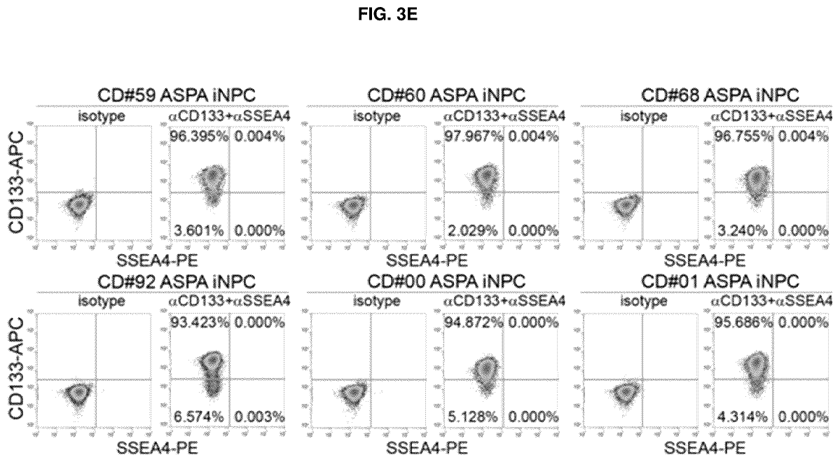

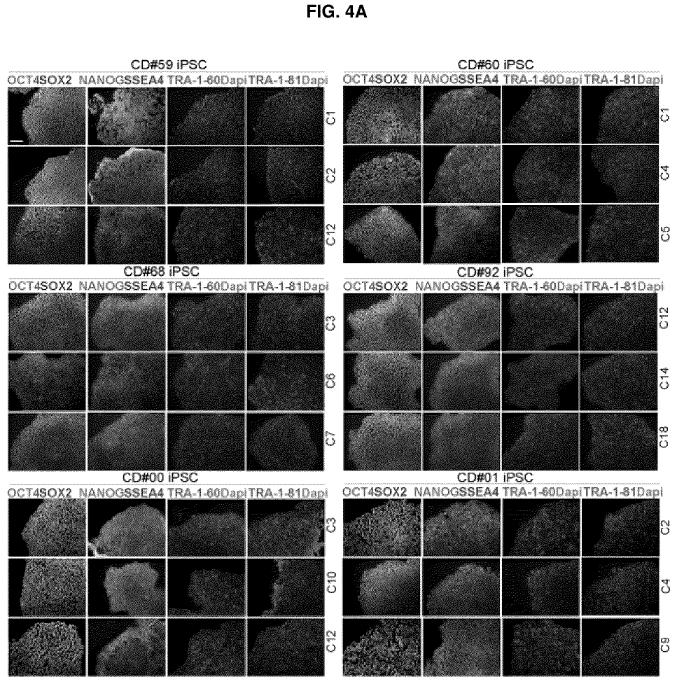

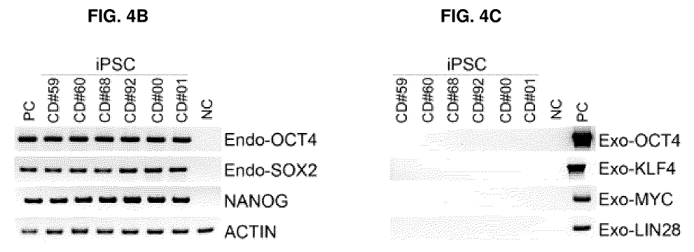

The iPSCs were derived from the fibroblasts generated from six CD patients using the GMP-compatible manufacturing process established. The cohort of the CD patients include patients CD #59 and CD #60 who carried the G176E and A305E mutations in the ASPA gene, patient CD #68 who carried the E285A mutation in the ASPA gene, patient CD #92 who had one nucleotide insertion in exon 2 of the ASPA gene, CD #00 who had a H244R mutation in the ASPA gene, and CD #01 who had a deletion and two point mutations in the ASPA gene (FIG. 3A). Among the ASPA mutations, A305E is the most common mutation (60%) in non-Jewish CD patients [13], while E285A is the predominant mutation (accounting for over 82% of mutations) among the Ashkenazi Jewish population [14]. The CD patient-derived fibroblast cells were reprogrammed via nucleofection to generate iPSCs using episomal vectors encoding the reprogramming factors human OCT4, SOX2, KLF4, L-MYC and LIN28. At least three iPSC colonies with typical human embryonic stem cell (ESC) morphology and marker expression (FIG. 4A) were selected and expanded for each patient fibroblast line.

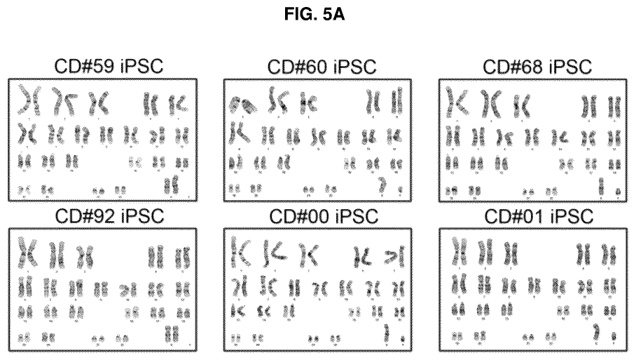

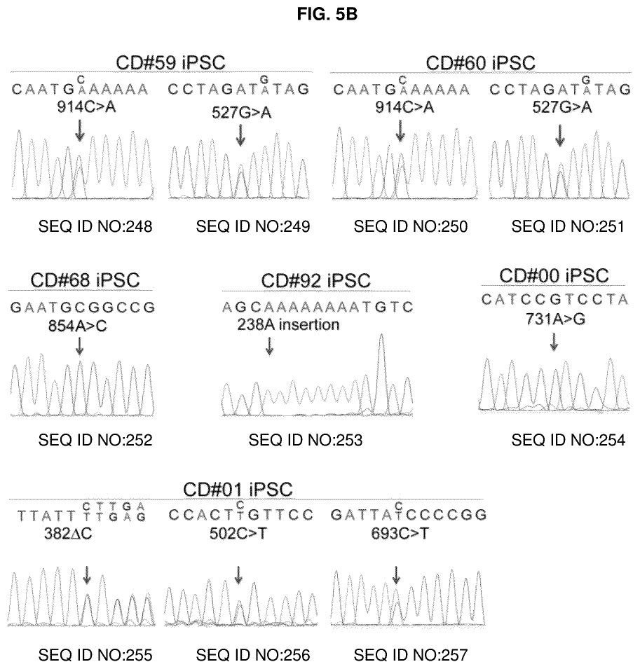

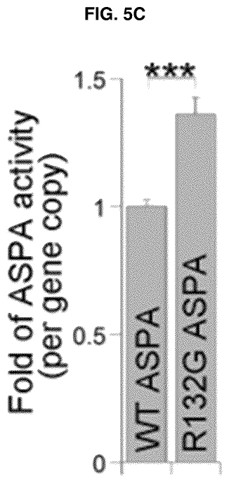

For each patient, one line of iPSCs that expressed the pluripotency genes and human ESC surface markers (FIG. 4A) and exhibited normal karyotype (FIG. 5A) was selected for in-process testing. All six lines were negative for microbial and mycoplasma contamination (Table 5).

TABLE 5
Characterization of CD iPSCs

| Tests | Method | Specification |
|---|---|---|
| Sterility | USP Sterility | No growth |
| Mycoplasma | Luminescence assay | Negative |
| Karyotype | G-banding | Normal |
| STR assay | PCR | 100% identity |
| Purity | Flow cytometry | >90% SSEA4⁺ cells |
| Residual exogenous | PCR | Not detectable |
| reprogramming factors | | |

STR analysis confirmed that all CD iPSC clones exhibited the same STR pattern as their parental fibroblast cells on all loci tested (Table 6).

TABLE 6
CD iPSCs Exhibit the Same STR Pattern as Parental Fibroblasts

| Locus | CD59 | | CD60 | | CD68 | | CD92 | | CD00 | | CD01 | |
|---|---|---|---|---|---|---|---|---|---|---|---|---|
| /lines | Fib | iPSC | Fib | iPSC | Fib | iPSC | Fib | iPSC | Fib | iPSC | Fib | iPSC |
| AMEL | X | X | X, | X, Y | X. | X, Y | X | X | X, | X, Y | X, Y | X, Y |
| | | | Y | | Y | | | | Y | | | |
| CSF1PO | 11 | 11, | 11 | 11 | 11 | 11 | 10 | 10, | 10, | 10 | 12, | 12, |
| | 12 | 12 | | | | | 12 | 12 | 11 | 11 | 13 | 13 |
| D13S317 | 8, | 8,11 | 8, | 8,11 | 11, | 11, | 12 | 12 | 11, | 11, | 11, | 11, |
| | 11 | | 11 | | 12 | 12 | | | 13 | 13 | 12 | 12 |
| D16S539 | 11, | 11, | 12 | 12 | 10, | 10 | 12 | 12 | 12 | 12 | 9, | 9, 10 |
| | 12 | 12 | | | 13 | 13 | | | | | 10 | |
| D21S11 | 30 | 30, | 30, | 30, | 29, | 29 | 29 | 29, | 30 | 30 | 30, | 30, |
| | 31.2 | 31.2 | 31.2 | 31.2 | 31.2 | 31.2 | 30 | 30 | | | 31 | 31 |
| D5S818 | 10, | 10, | 11, | 11, | 10, | 10, | 9, 12 | 9, 12 | 12, | 12, | 12 | 12 |
| | 12 | 12 | 12 | 12 | 12 | 12 | | | 13 | 13 | | |
| D7S820 | 9, | 9, 11 | 11 | 11 | 9, | 9, 12 | 10 | 10, | 11 | 11 | 10 | 10 |
| | 11 | | | | 12 | | 11 | 11 | | | | |
| TH01 | 9.3 | 9.3 | 6, 9.3 | 6, 9.3 | 9, 9.3 | 9, 9.3 | 7 | 7 | 6, 7 | 6, 7 | 7, 9.3 | 7, 9.3 |
| TPOX | 8, | 8, 11 | 8, | 8, 11 | 9, | 9,11 | 8 | 8 | 8 | 8 | 8, | 8, 12 |
| | 11 | | 11 | | 11 | | | | | | 12 | |
| vWA | 14, | 14, | 14, | 14, | 17, | 17, | 16,17 | 16,17 | 18 | 18 | 14, | 14, |
| | 17 | 17 | 18 | 18 | 18 | 18 | | | | | 17 | 17 |

Note: If both alleles at a locus have the same STIR genotype, only one X or number is shown.

For each CD patient iPSC line, flow cytometry analysis showed that more than 90% cells express the pluripotency marker 0014 and the human ESC surface marker SSEA4 (Table 7).

TABLE 7
Flow Cytometry Analysis of CD iPSCs

| | HEK | H9 | CD59 | CD60 | CD68 | CD92 | CD00 | CD01 |
|---|---|---|---|---|---|---|---|---|
| % Cells | 293T | ESC | iPSC | iPSC | iPSC | iPSC | iPSC | iPSC |
| % | −0.008 | 98.95 | 98.26 | 98.47 | 98.08 | 97.80 | 96.99 | 98.06 |
| OCT4⁺ | | | | | | | | |
| cells | | | | | | | | |
| % | 0.011 | 98.96 | 98.86 | 93.26 | 99.29 | 99.83 | 99.55 | 97.64 |
| SSEA4⁺ | | | | | | | | |
| cells | | | | | | | | |

RT-PCR analysis was performed to confirm the activation of the endogenous pluripotency genes and detect any residual exogenous reprogramming factors in each CD iPSC line. The activation of the endogenous OCT4, SOX2, and NANOG gene expression was detected in iPSCs derived from each CD patient fibroblast line, whereas the exogenous reprogramming factors, OCT4, KLF4, MYC, and LIN28, were not detectable in any iPSCs by passage 6 (FIGS. 4B, 4C). Sanger sequencing confirmed that each CD patient-derived iPSC line harbored the same ASPA mutation as the corresponding CD patient (FIG. 5B).

After in-process testing, the CD iPSCs that met the specifications were differentiated into CD iNPCs. The CD iNPCs lines were expanded up to passage 6. At this stage, all CD iNPC lines were tested for sterility and mycoplasma and confirmed to be free of contamination.

### Example 3: Generating ASPA iNPCs by Lentiviral Transduction of a Functional ASPA Gene into CD iNPCs

Because CD is caused by ASPA gene mutations, which lead to deficient ASPA enzymatic activity, a functional ASPA gene was introduced into CD iNPCs by transducing CD iNPCs with a lentiviral vector. The lentiviral vector consisting of the sequence of a functional human ASPA gene (R132G ASPA) under the control of the constitutive human EF1α promoter was called LV-EF1α-hASPA. The R132G mutation created outside of the catalytic center for the purpose of tracking did not disrupt the ASPA enzymatic activity, but increased ASPA activity mildly (FIG. 50). The LV-EF1α-hASPA was used for genetic modification of CD iNPCs. The resultant cellular product was termed ASPA

The ASPA iNPCs were sampled during manufacturing (in-process, Tables 5-7) and at final product stage (FIG. 3B and Table 8) for characterization.

TABLE 8
ASPA iNPCs Exhibit the Same STR Pattern as Parental Fibroblasts

| | CD59 | | CD60 | | CD68 | | CD92 | | CD00 | | CD01 | |
|---|---|---|---|---|---|---|---|---|---|---|---|---|
| | | ASP | | ASP | | ASP | | ASP | | ASPA | | ASPA |
| Locus | | A | | A | | A | | A | | | | |
| /lines | Fib | INPC | Fib | INPC | Fib | INPC | Fib | INPC | Fib | INPC | Fib | INPC |
| AMEL | X | X | X, Y | X, Y | X, Y | X, Y | X | X | X, Y | X, Y | X, Y | X, Y |
| CSF1PO | 11, | 11, | 11 | 11 | 11 | 11 | 10, | 10, | 10, | 10, 11 | 12, | 12, 13 |
| | 12 | 12 | | | | | 12 | 12 | 11 | | 13 | |
| D13S317 | 8, 11 | 8, 11 | 8, 11 | 8, 11 | 11, | 11, | 12 | 12 | 11, | 11, 13 | 11, | 11, 12 |
| | | | | | 12 | 12 | | | 13 | | 12 | |
| D16S539 | 11, | 11, | 12 | 12 | 10, | 10, | 12 | 12 | 12 | 12 | 9, | 9, 10 |
| | 12 | 12 | | | 13 | 13 | | | | | 10 | |
| D21S11 | 30, | 30, | 30, | 30, | 29, | 29, | 29, | 29 | 30 | 30 | 30, | 30, 31 |
| | 31.2 | 31.2 | 31.2 | 31.2 | 31.2 | 31.2 | 30 | 30 | | | 31 | |
| D5S818 | 10, | 10, | 11, | 11, | 10, | 10, | 9, | 9, 12 | 12, | 12, 13 | 12 | 12 |
| | 12 | 12 | 12 | 12 | 12 | 12 | 12 | | 13 | | | |
| D7S820 | 9, 11 | 9, 11 | 11 | 11 | 9, 12 | 9, 12 | 10, | 10, | 11 | 11 | 10 | 10 |
| | | | | | | | 11 | 11 | | | | |
| TH01 | 9.3 | 9.3 | 6, | 6, | 9, | 9, | 7 | 7 | 6, 7 | 6, 7 | 7, | 7, 9.3 |
| | | | 9.3 | 9.3 | 9.3 | 9.3 | | | | | 9.3 | |
| TPOX | 8, 11 | 8, 11 | 8, 11 | 8, 11 | 9, 11 | 9, 11 | 8 | 8 | 8 | 8 | 8, | 8, 12 |
| | | | | | | | | | | | 12 | |
| vWA | 14, | 14, | 14, | 14, | 17, | 17, | 16, | 16, | 18 | 18 | 14, | 14, 17 |
| | 17 | 17 | 18 | 18 | 18 | 18 | 17 | 17 | | | 17 | |

Note: If both alleles at a locus have the same STR genotype, only one X or number is shown.

According to the established procedures, the ASPA iNPCs were characterized for sterility, mycoplasma, viability at thaw, endotoxin, STR profiling, ASPA transgene copy #, ASPA activity, % NPC (CD133⁺SSEA4⁻ cells) and % residual iPSC (SSEA4⁺ cells by FACS and REX1⁺ cells by RT-qPCR). The copy number of the virally transduced ASPA transgene in the ASPA iNPCs was determined by TaqMan real time PCR following a published protocol [15]. The copy number of the transgene is less than five in all 6 ASPA iNPC lines. The ASPA activity was measured using a coupled enzymatic reaction [16] and robust ASPA activity was detected in each ASPA iNPC line (FIG. 3B).

The ASPA iNPCs were also characterized to confirm that they expressed typical NPC markers PAX6, SOX1, NESTIN and CD133. All 6 lines of ASPA iNPC lines expressed typical NPC markers, including NESTIN, SOX1, and PAX6, as revealed by immunostaining (for NESTIN and SOX1) and RT-PCR (for SOX1 and PAX6) analyses (FIGS. 3C, 3D), whereas no expression of the pluripotency factors OCT4 and NANOG was detected in the ASPA iNPCs (FIG. 3D). The FACS analysis was performed to determine the percentage of CD133⁺SSEA4⁻ NPC population, which ranged from 93.42% to 97.97% in six lines of ASPA iNPCs, and the lack of residual iPSCs in ASPA iNPCs (0 to 0.004% by SSEA4 FACS and <0.003% by REX1 RT-qPCR) was confirmed (FIG. 3E). The ASPA iNPCs derived from 6 CD patients all met the release testing criteria. Accordingly, the GMP-compatible manufacturing processes were established and the genetically modified ASPA iNPCs were generated from CD patients using these processes.

### Example 4: Generation of Immunodeficient CD (Nur7) Mice for ASPA iNPC Transplantation

The Aspa^{nur7/nur7} mouse contains a nonsense mutation (Q193X) in the ASPA gene [17]. Because the Aspa^{nur7/nur7} mice exhibit key pathological phenotypes resembling those of CD patients, including loss of ASPA enzymatic activity, elevated NAA levels, and extensive spongy degeneration in various brain regions [17], it is considered a relevant animal model for CD. Therefore, the Aspa^{nur7/nur7} mouse provides an excellent platform for testing the therapeutic effects of the ASPA iNPCs.

Because transplanting human cells into CD (Nur7) mice was needed, an immunodeficient ASPA^{nur7/nur7} mouse model was generated by breeding the Aspa^{nur7/nur7} mice with immunodeficient Rag2^{-/-} mice, which lacked mature B and T lymphocytes [18]. The resultant Aspa^{nur7/nur7}/Rag2^{-/-} mice were termed “CD (Nur7) mice” for short. These mice exhibited a range of pathological features of CD (see results below) and were used for transplantation studies to evaluate the efficacy of the ASPA iNPC cellular product. All CD (Nur7) mice used for transplantation were verified to carry homozygous nur7 and Rag2 genetic mutations by genotyping. Postnatal day (PND) 1-4 pups of both sexes were used for transplantation.

### Example 5: The Distribution and Cell Fate of ASPA iNPCs in the Transplanted CD (Nur7) Mouse Brains

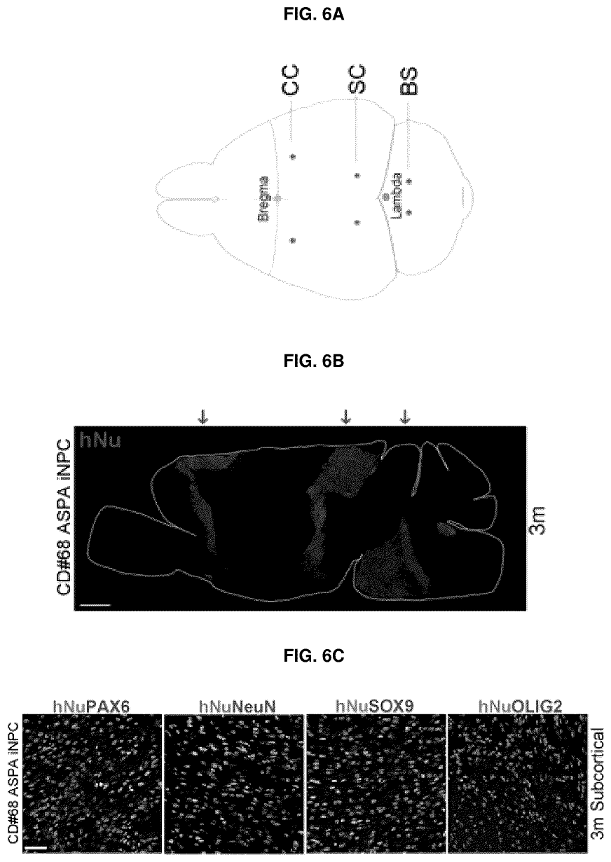

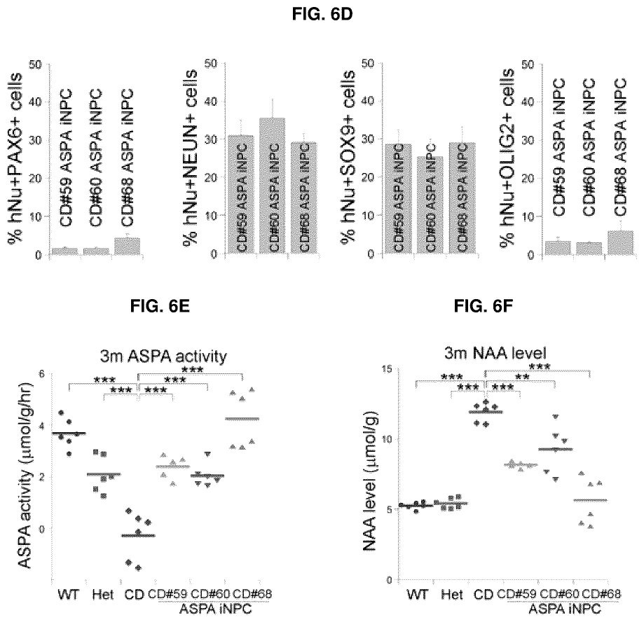

Three lines of ASPA iNPCs derived from three different CD patients, including CD #59, CD #60, and CD #68, were injected into CD (Nur7) mouse brains individually. The injection was performed bilaterally into six sites. The injection sites include the corpus callosum, the subcortical white matter, and the brain stem (FIG. 6A). The ASPA iNPC-transplanted mice were evaluated at organismal, histological, and biochemical levels. The wild type (WT, ASPA^{+/+}/Rag2^{-/-}) and/or heterozygous (Het, ASPA^{nur7/+}/Rag2^{-/-}) mice were included as the positive control, while the un-transplanted CD (Nur7) mice (ASPA^{nur7/nur7}/Rag2^{-/-}) were included as the negative control for the preclinical efficacy studies. In addition, the medium for ASPA iNPCs was injected into CD (Nur7) mouse brains using the same coordinates and procedure as for cell transplantation as a sham control.

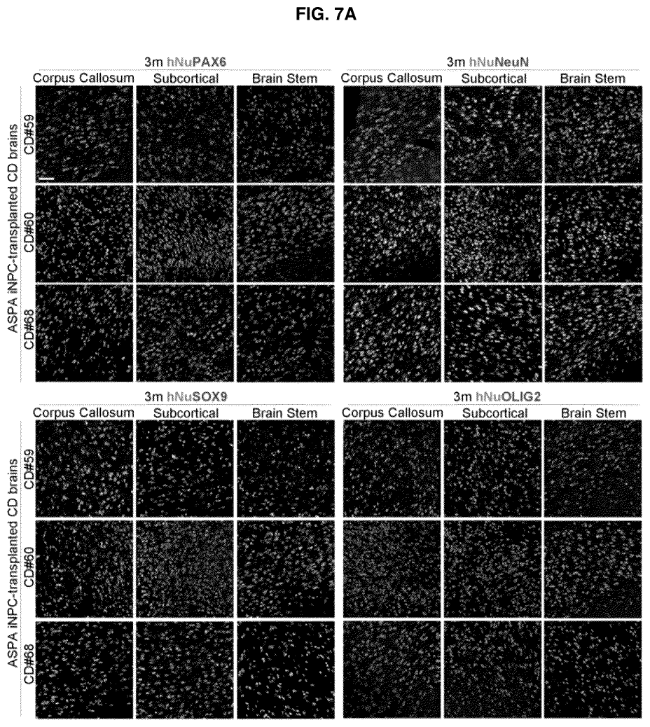

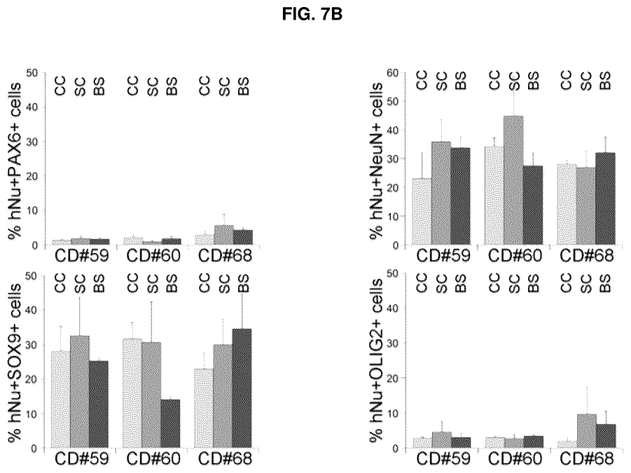

First, the survival, distribution and cell fate of the ASPA iNPCs in brains of the transplanted mice were determined by immunohistochemical staining for human nuclear antigen (hNu) and markers of various neural lineage cells. Three months after transplantation, brains of the transplanted mice were harvested. The survival of the transplanted ASPA iNPCs was determined by immunostaining the transplanted mouse brains for hNu. The signal of hNu was detected in multiple regions of the transplanted brain, including the corpus callosum, the subcortical region, and the brain stem region (FIG. 7A). The ASPA iNPCs were distributed around the injection sites, without extensive migration, in the transplanted CD (Nur7) mouse brain (FIG. 6B).

Double staining of the transplanted brains with antibodies for hNu and the NPC marker PAX6 revealed that a small portion of the ASPA iNPCs was maintained as NPCs (FIGS. 6C, 6D, 7A, and 7B). Double staining for hNu and the neuronal marker NeuN, the astrocyte marker SOX9, and the oligodendroglial lineage marker OLIG2, respectively, revealed that the ASPA iNPCs could give rise to neurons, astrocytes, and oligodendroglial lineage cells in the transplanted brains (FIGS. 6C, 6D, 7A, and 7B). There was no obvious difference in the fate of the transplanted cells in the regions where they were located, including the corpus callosum, the subcortical and the brain stem white matters (FIGS. 6C, 6D, 7A, and 7B), presumably because they were all white matter tracks.

### Example 6: Increased ASPA Activity and Reduced NAA Levels in ASPA iNPC-Transplanted CD (Nur7) Mouse Brains

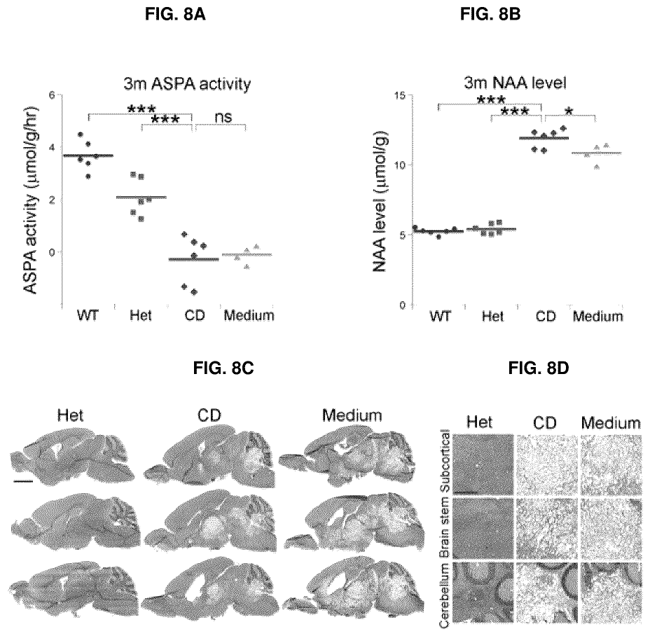

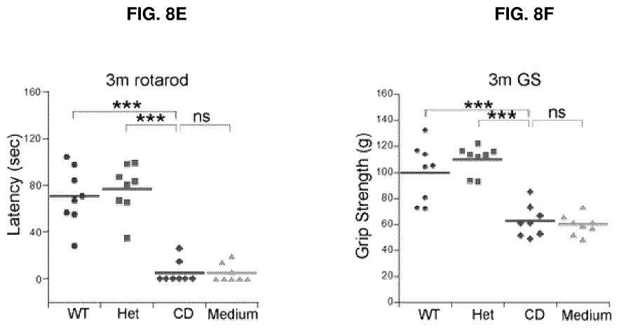

Because the deficiency in ASPA enzymatic activity is the underlying cause of disease phenotypes in both CD patients and animal models, the ASPA enzymatic activity in ASPA iNPC-transplanted CD (Nur7) mouse brains was determined. Three months after transplantation, brains of the ASPA iNPC-transplanted mouse brains were evaluated for ASPA enzymatic activity and NAA levels. Potent ASPA enzymatic activity was detected in brains of all ASPA iNPC-transplanted mice, compared to that in control CD (Nur7) mouse brains without transplantation (FIG. 6E). In contrast, the medium-treated CD (Nur7) mice exhibited deficient ASPA activity, similar to the control CD (Nur7) mice (FIG. 8A). Further comparison revealed that the ASPA activity in the ASPA iNPC-transplanted CD (Nur7) mouse brains was similar to or higher than the ASPA activity in the Het mice. Of interest, both Het human subjects and Het CD (Nur7) mice were phenotypically normal [17], although the ASPA activity in the Het mouse brains was about 50-60% of that in the WT brains (FIG. 6E). It has been shown that ASPA deficiency leads to elevated NAA level in brains of both CD patients and mouse models [1, 17, 19]. Consistent with elevated ASPA enzymatic activity, reduced NAA level was detected in the ASPA iNPC-transplanted CD (Nur7) mouse brains, compared to that in control CD (Nur7) mouse brains (FIG. 6F). In contrast, the NAA level remained to be elevated in medium-treated CD (Nur7) mouse brains (FIG. 8B). These results together indicate that transplantation with the ASPA iNPCs was able to rescue the deficiency of ASPA enzymatic activity and reduce NAA level, both of which are major defects in CD patients and mouse models, and that the therapeutic effect was resulted from the cell products instead of the procedure by itself because medium control exhibited no effect on either ASPA activity or NAA level.

### Example 7: Rescue of Spongy Degeneration in ASPA iNPC-Transplanted CD (Nur7) Mouse Brains

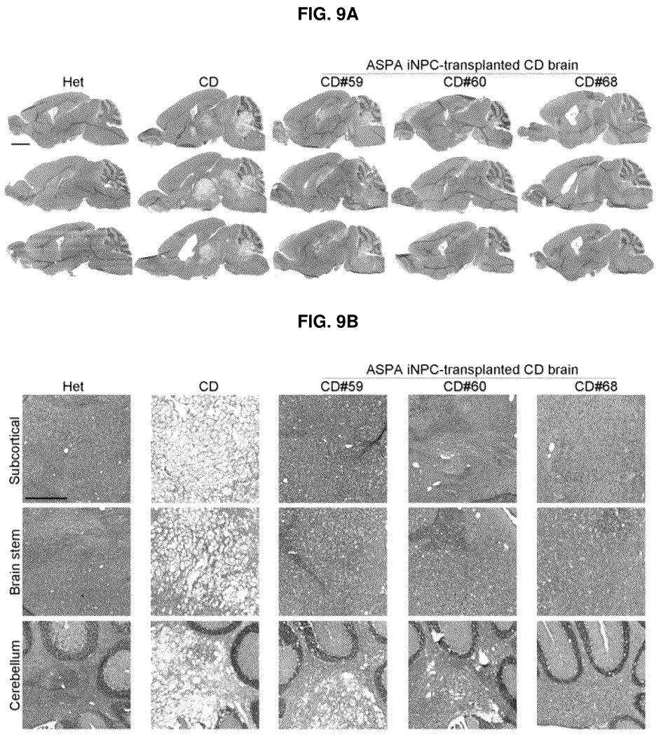

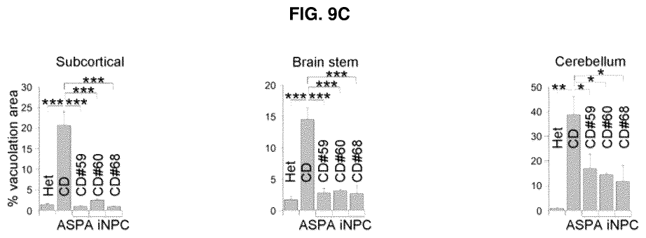

Extensive spongy degeneration is a key pathological feature of CD patients and mouse models, which is revealed by vacuolation in various brain regions [1, 17, 19]. Indeed, extensive vacuolation was observed in brains of the CD (Nur7) mice, compared to brains of the Het mice, which had intact brain parenchyma (FIGS. 9A-9C). In contrast, H&E staining revealed substantially reduced vacuolation in various brain regions of the ASPA iNPC-transplanted CD (Nur7) mice, including the subcortical white matter, the brain stem and the cerebellum (FIGS. 9A-9C), but not in medium-treated CD (Nur7) mice (FIGS. 8C, 8D).

The extent of rescue in the cerebellum region was not as extensive as the subcortical white matter and the brain stem regions, presumably because the cerebellum is too far away from the injection sites. The ASPA iNPCs derived from three different CD patients all led to substantial rescue, in a comparable manner (FIGS. 9A-9C). These results indicate that transplantation with the ASPA iNPCs was able to rescue the spongy degeneration phenotype in CD (Nur7) mouse brains, supporting the therapeutic potential of the ASPA iNPCs for their ability to ameliorate the pathological phenotypes of CD.

### Example 8: Improved Myelination in ASPA iNPC-Transplanted CD (Nur7) Mouse Brains

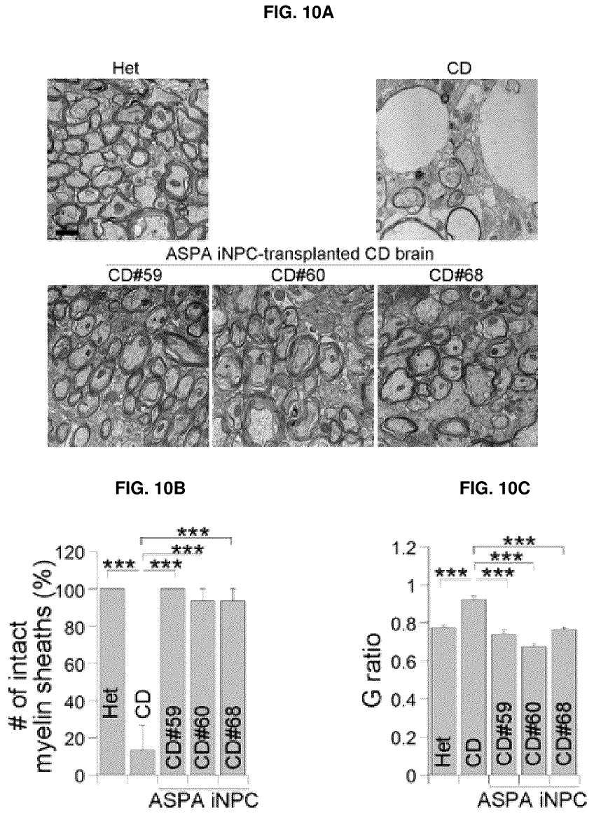

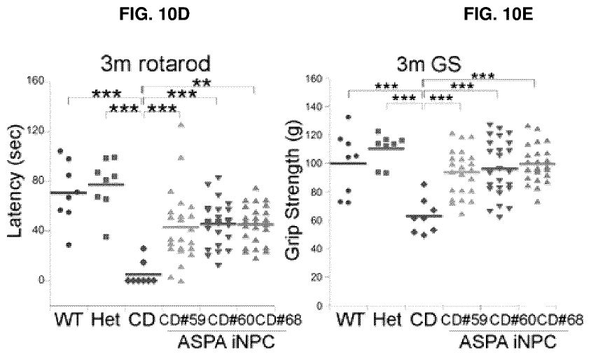

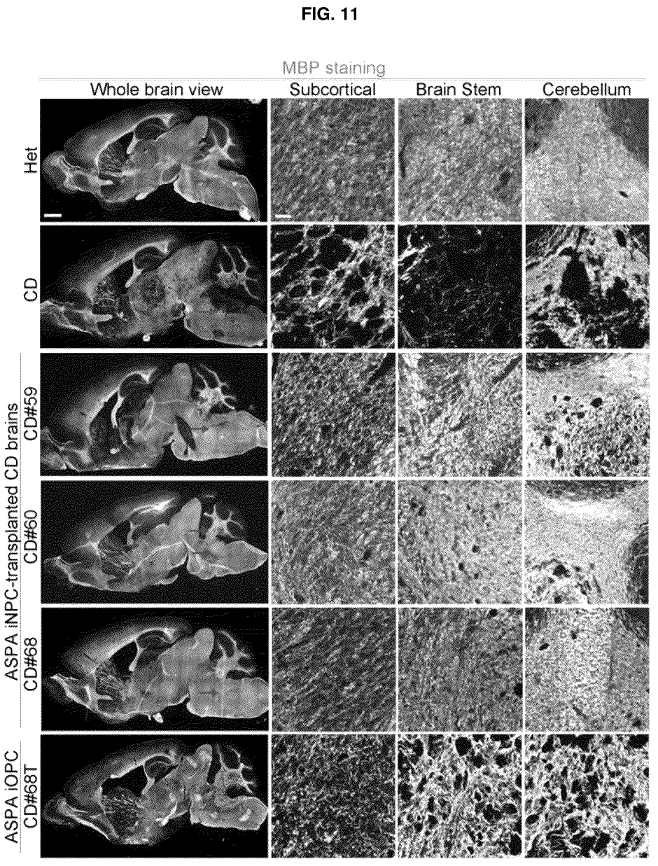

It has been suggested that vacuolation results from myelin destruction in brains of CD (Nur7) mice [17]. Consistent with the extensive vacuolation detected in brains of the CD (Nur7) mice, substantially reduced number of normal myelin sheaths was observed in brains of the CD (Nur7) mice, compared to that of the Het mice, as revealed by electron microscopy (EM) analysis (FIGS. 10A, 10B) and MBP staining (FIG. 11). G ratio, the ratio of the inner diameter to the outer diameter of myelin sheaths, was also altered in CD (Nur7) mouse brains. Increased G ratio was detected in brains of the CD (Nur7) mice, compared to that in the heterozygous mice (FIGS. 10A, 10C). Transplantation with ASPA iNPCs led to substantially improved myelination in CD (Nur7) mouse brains. The number of normal myelin sheaths in the ASPA iNPC-transplanted CD brains was much higher than that in the control CD brains, reaching the level in the Het mouse brains (FIGS. 10A, 10B). Moreover, the G ratio of myelin sheaths in the transplanted brains resembled that in the Het mouse brains, both of which were much lower than that in that in the control CD brains (FIGS. 10A, 10C), indicating that the myelin sheaths in the transplanted brains are thicker than those in the untreated control CD brains. The reduced myelin sheaths and disordered nerve tracts could also be found in CD (Nur7) mouse brains by immunostaining for MBP, a marker of myelination (FIG. 11). Transplantation with the ASPA iNPCs improved myelination as revealed by enhanced MBP staining and better-organized nerve tracks (FIG. 11).

### Example 9: Rescue of Gross Motor & Neuromuscular Function in ASPA iNPC-Transplanted CD (Nur7) Mice

Defect in motor performance is typical of CD patients and animal models [1, 17, 19]. To determine if transplantation with the ASPA iNPCs could rescue the defective motor performance in CD (Nur7) mice, the ASPA iNPC-transplanted CD (Nur7) mice were tested in two motor skill paradigms at 3 months after transplantation. First, the transplanted mice were tested using an accelerating rotarod, a device that is designed for testing motor coordination and balance [20]. Transplantation with ASPA iNPCs improved rotarod performance substantially in CD (Nur7) mice transplanted with any of the three ASPA iNPC lines, compared to the control CD (Nur7) mice (FIG. 10D). A grip strength test was performed to evaluate the forepaw strength as an indication of neuromuscular function [21], using a grip strength meter. Substantial enhancement of the grip strength was also detected in CD (Nur7) mice, compared to that in the control CD (Nur7) mice transplanted with any of the three lines of ASPA iNPCs (FIG. 10E). In contrast, treatment with the medium control exhibited no effect on either the rotarod performance or the grip strength of the CD (Nur7) mice (FIGS. 8E, 8F). These results indicate that the ASPA iNPCs can substantially improve motor functions in a mouse model of CD. These results together provide a proof-of-concept that the ASPA iNPCs have great therapeutic potential to ameliorate the pathological phenotypes of CD.

### Example 10: Sustained Rescue of Disease Phenotypes in ASPA iNPC-Transplanted CD (Nur7) Mice

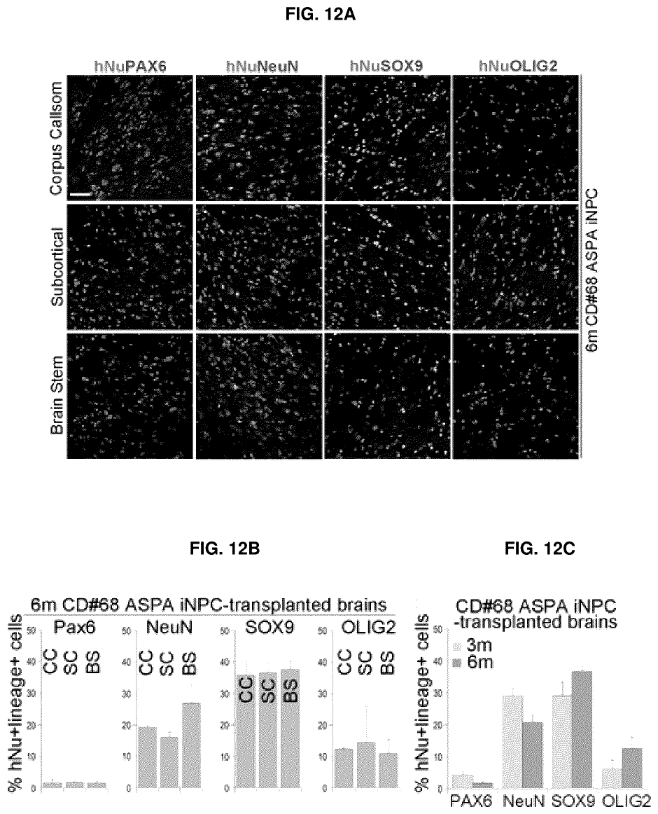

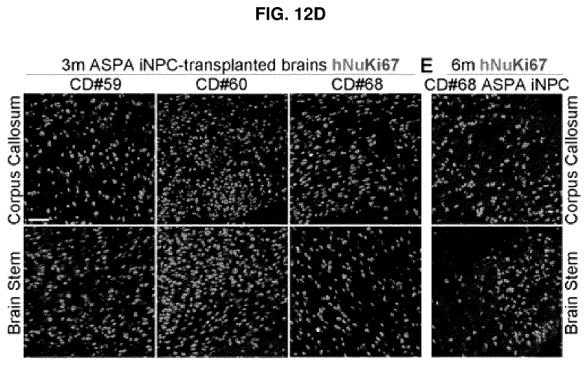

The ASPA iNPCs were sustained in brains of the transplanted mice 6 months after transplantation and the cell fate was largely maintained (FIGS. 12A, 12B), although there was a mild increase in the astrocyte (hNu⁺SOX9⁺) and the oligodendroglial (hNu⁺OLIG2⁺) populations, and a mild reduction in the NPC (hNu⁺PAX6⁺) and neuronal (hNu⁺NeuN⁺) populations from the transplanted cells 6 months post-transplantation, compared to 3 months post-transplantation (FIG. 12C).

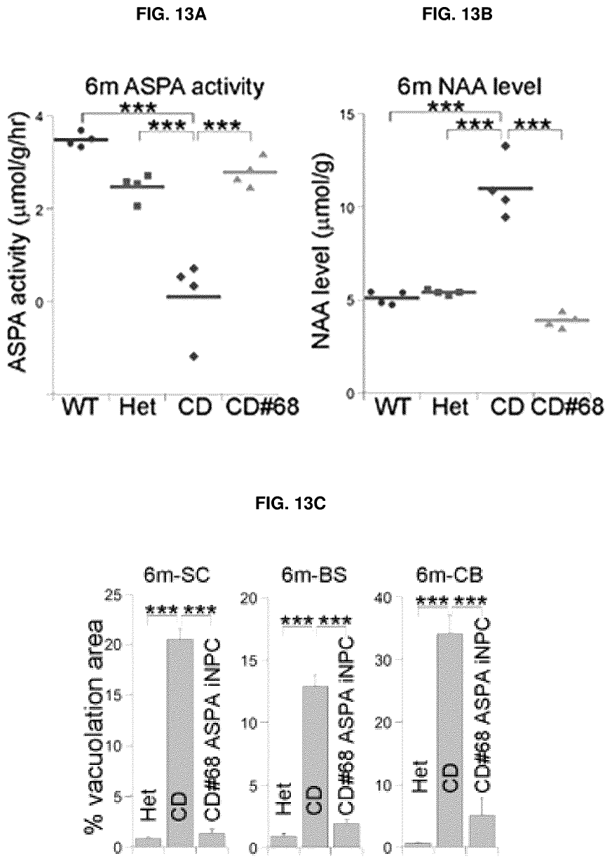

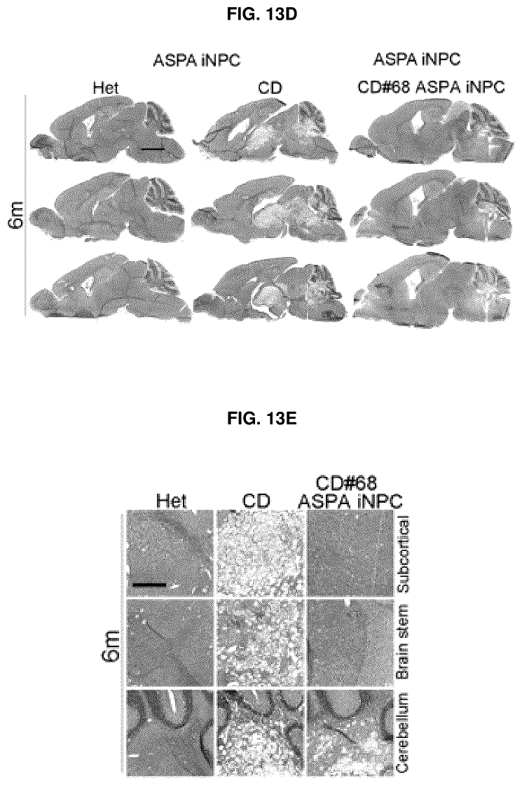

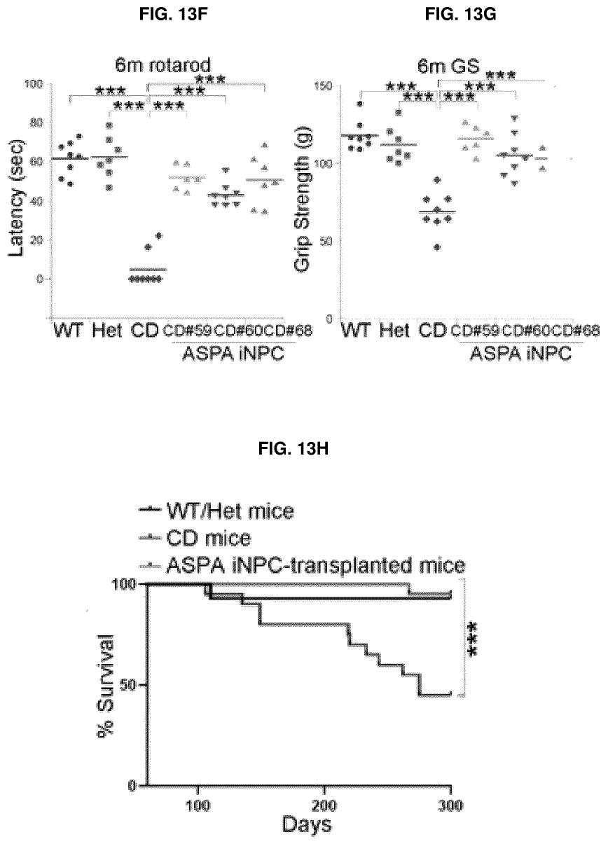

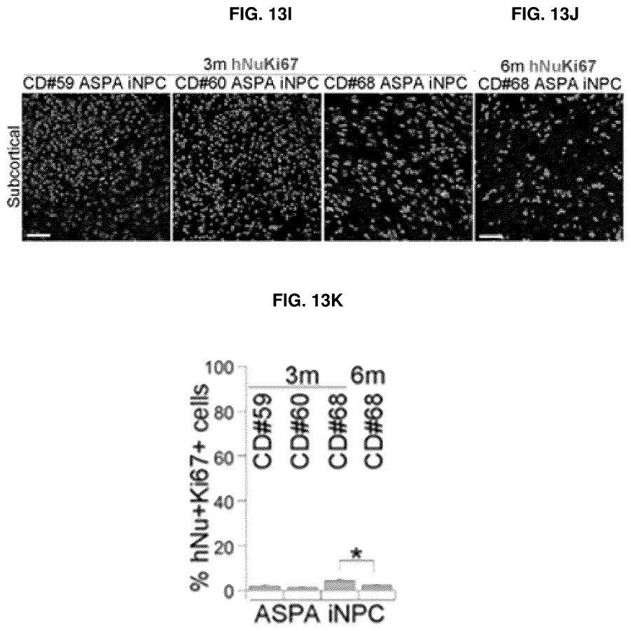

To determine if transplantation with the ASPA iNPCs could lead to sustained ASPA activity, the brains of the CD68 ASPA iNPC-transplanted CD (Nur7) mouse brains were evaluated for ASPA activity six months after transplantation. Substantially higher ASPA enzymatic activity was detected in brains of ASPA iNPC-transplanted CD (Nur7) mice, compared to that in control CD (Nur7) mice (FIG. 13A). The ASPA activity in the ASPA iNPC-transplanted CD (Nur7) mouse brains is similar to or even slightly higher than the ASPA activity in the Het mice 6 months after transplantation (FIG. 13A). Consistent with elevated ASPA enzymatic activity, dramatically reduced NAA level was detected in the brains of ASPA iNPC-transplanted CD (Nur7) mice, compared to that in control CD (Nur7) mice (FIG. 13B). These results together indicate that transplantation with ASPA iNPCs was able to rescue the deficiency of ASPA enzymatic activity and reduce NAA level in a sustainable manner.

To determine if ASPA iNPC transplantation could have long-term beneficial effect, the brains of the ASPA iNPC-transplanted CD (Nur7) mice were examined for vacuolation. Substantially reduced vacuolation in various brain regions of the CD #68 ASPA iNPC-transplanted CD (Nur7) mice, including the subcortical white matter, the brain stem and the cerebellum, was detected 6 months after transplantation (FIGS. 13C-13E). These results indicate that transplantation with the ASPA iNPCs was able to rescue the spongy degeneration phenotype in CD (Nur7) mouse brains in a sustainable manner.

To determine if transplantation with the ASPA iNPCs could lead to sustained improvement of motor function in CD (Nur7) mice, the ASPA iNPC-transplanted CD (Nur7) mice were tested at 6 months after transplantation. The ASPA iNPCs improved rotarod performance in transplanted CD (Nur7) mice substantially 6 months after transplantation, compared to the control CD (Nur7) mice (FIG. 13F). Considerable enhancement of the grip strength was also detected in the ASPA iNPC-transplanted CD (Nur7) mice 6 months after transplantation, compared to the control CD (Nur7) mice (FIG. 13G). This result indicates that the engrafted ASPA iNPCs can sustain improved motor functions in CD (Nur7) mice.

### Example 11: The ASPA iNPC-Transplanted Mice Exhibit Prolonged Survival

The ASPA iNPC-transplanted CD (Nur7) mice were monitored for up to 10 months to track their life span. The WT and Het mice were included as the positive control and the CD (Nur7) mice as the negative control. Substantially prolonged lifespan in the ASPA iNPC-transplanted mice was observed, compared to the control CD (Nur7) mice (FIG. 13H). While 45% of the control CD (Nur7) mice (n=20) died before 10 months, only one ASPA iNPC-transplanted CD (Nur7) mouse out of a total of 20 transplanted mice died before 10 months. Taken together, the results from the preclinical efficacy study provide a proof-of-concept that the ASPA iNPCs have great therapeutic potential to ameliorate the pathological phenotypes of CD in a robust and sustainable manner.

### Example 12: Preliminary Safety of the ASPA iNPCs in the Transplanted CD (Nur7) Mice

For a preliminary safety study, CD (Nur7) mice transplanted with the ASPA iNPCs were monitored monthly for up to 10 months, and no signs of tumor formation or other adverse effects were observed. At the end of 3 and 6 months, the brains of the transplanted mice were harvested and analyzed. No tumor tissue was found in the transplanted brain sections. The lack of tumor formation in the ASPA iNPC-transplanted brains was confirmed by Ki67 staining. A low mitotic index, as revealed by the low percentage (1.35% to 4.32%) of hNu and Ki67 double positive (hNu⁺Ki67⁺) cells out of total hNu⁺ cells, was detected in the ASPA iNPC-transplanted brains at both 3 months and 6 months post-transplantation (FIGS. 13I-13K, 12D). Furthermore, although separate animal brains were observed at 3- and 6-months after transplantation, the percent of hNu⁺Ki67⁺ cells out of total hNu⁺ cells appeared not to increase but to actually decrease in the transplanted brains, from 4.32% (at 3 months) to 2.20% (at 6 months) in CD #68 ASPA iNPC-transplanted brains (FIG. 13K). These results demonstrate preliminary safety of ASPA iNPCs in transplanted brains.

### Example 13: The ASPA iOPCs Exhibit Widespread Distribution in Transplanted CD (Nur7) Mice

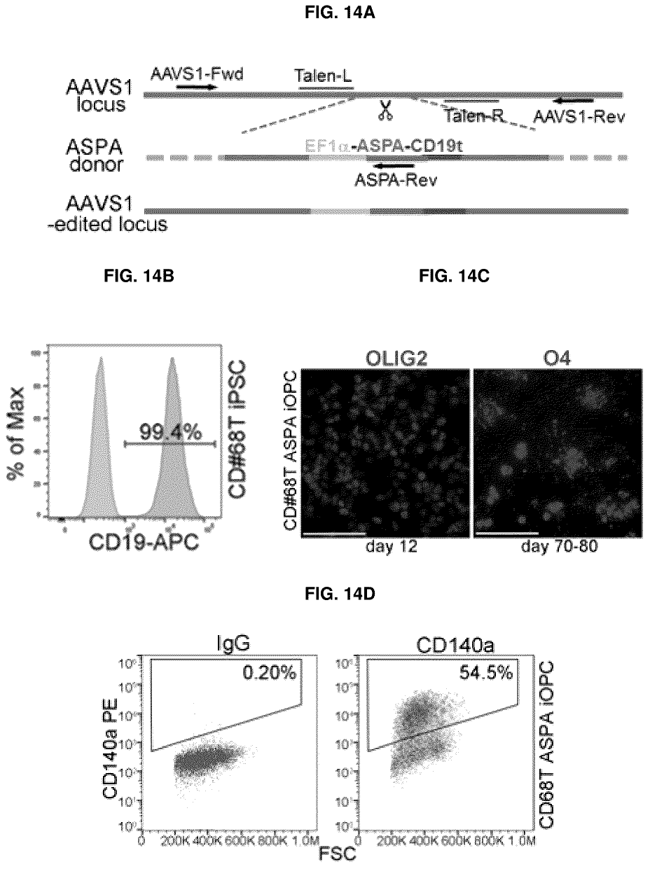

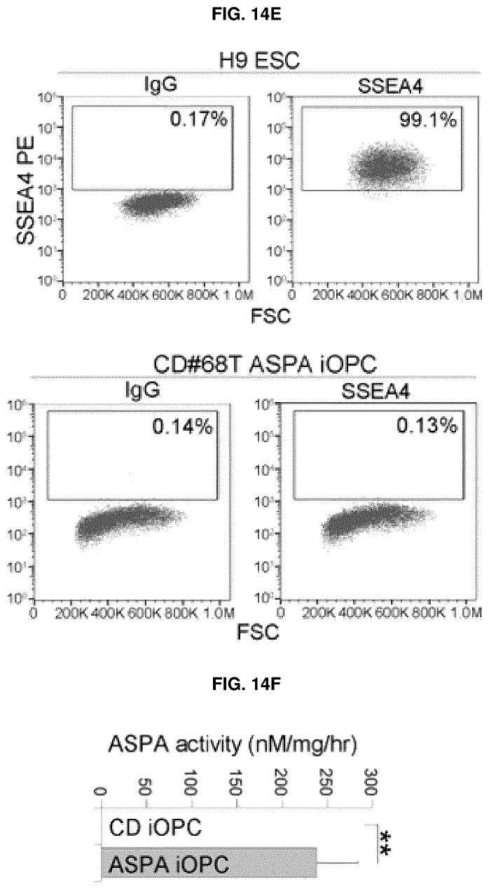

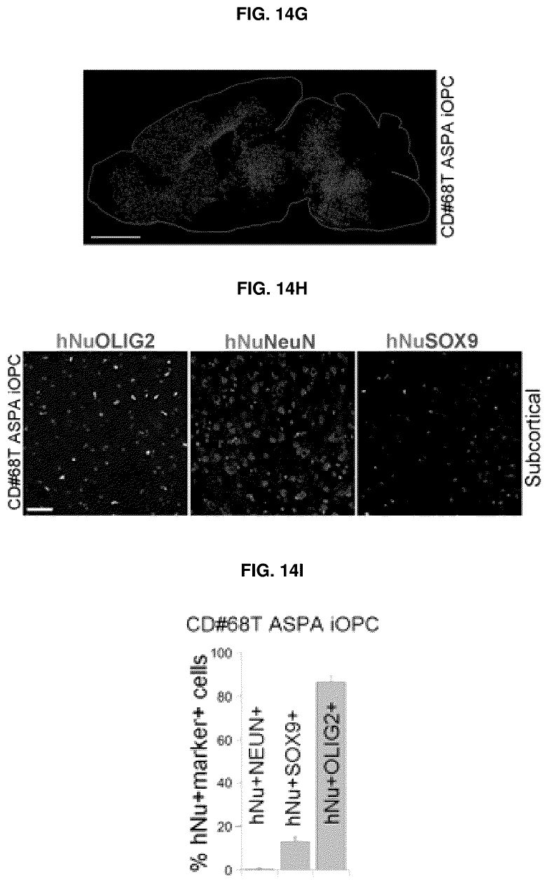

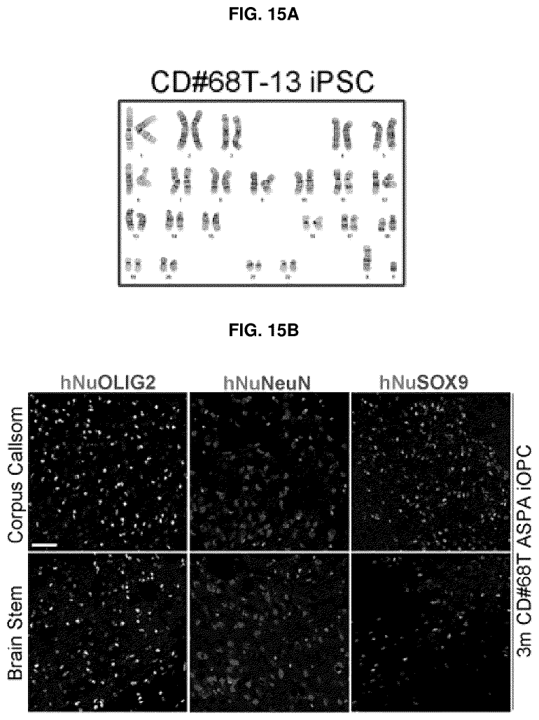

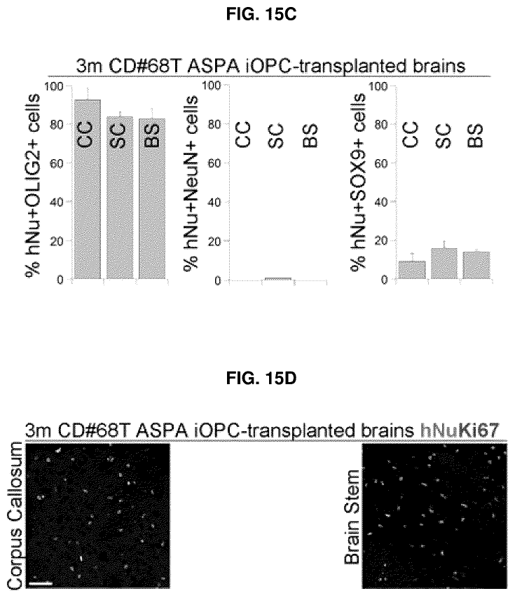

As an alternative to introducing a functional ASPA gene into CD iNPCs through lentiviral transduction, a WT ASPA gene was also knocked in into the AAVS1 safe harbor site in CD68 iPSCs through TALEN-mediated gene editing (FIG. 14A). The WT ASPA gene was linked to a truncated CD19 (CD19t) surface marker through T2A. The gene-edited iPSCs were selected by flow cytometry using a CD19-specific antibody. The single cell-derived colonies were picked and expanded. One of the colonies, CD68T-13 ASPA iPSCs, was chosen for further analysis based on colony morphology. Flow cytometry analysis using a CD19-specific antibody confirmed that the CD68T-13 ASPA iPSC colony contained more than 99% ASPA-CD19t-positive cells (FIG. 14B), confirming successful knock-in. The CD68T-13 ASPA iPSCs exhibited normal karyotype (FIG. 15A) and lacked off-target mutation as revealed by whole genome sequencing (Table 2).

Next the CD68T-13 ASPA iPSCs were differentiated into iOPCs following a published protocol [10c, 10d]. The ASPA iPSCs were first differentiated into OLIG2+ pre-OPCs, followed by induction into O4+ OPCs (FIG. 14C). These ASPA iPSC-derived OPCs were termed ASPA iOPCs. Flow cytometry analysis revealed enrichment of CD140a (PDGFαR)⁺ OPCs (54.5%) in the differentiated cell population [22] (FIG. 14D). In contrast, the CD68T ASPA iOPCs contained no detectable SSEA4⁺ residual pluripotent stem cells (0.13% detected by SSEA4 antibody minus 0.14% by IgG control) (FIG. 14E). A pure population of ASPA iOPCs could be obtained by CD140a-directed magnetic-activated cell sorting. The ASPA iOPCs exhibited potent ASPA enzymatic activity, compared to control CD68 iOPCs without ASPA knock-in (FIG. 14F).

The ASPA iOPCs were then transplanted into brains of CD (Nur7) mice for efficacy evaluation using the same procedure as used for ASPA iNPC transplantation (FIG. 6A). The distribution and cell fate of the engrafted ASPA iOPCs were analyzed three months after transplantation. In contrast to the ASPA iNPCs, the ASPA iOPCs showed widespread distribution throughout the brain as evidenced by immunostaining with hNu at 3 months after transplantation (FIG. 14G). The ASPA iOPCs were detected in the forebrain, the subcortical and the brain stem regions, although not the cerebellum, which may be too far away from the injection sites. Co-staining for hNu and different cell lineage markers revealed that most donor cells were oligodendroglial lineage cells. The proportion of hNu⁺OLIG2⁺ cell reached 86.35±2.90%. The remaining transplanted cells mostly became astrocytes (12.92±1.97% hNu⁺SOX9⁺ cells). Only very few human cell-derived neurons were detected in the transplanted brain (0.33±0.33% hNu⁺NeuN⁺ cells) (FIGS. 14H, 14I, 15B, 15C). These results indicate that the ASPA iOPCs could migrate and gave rise to oligodendroglial lineage cells in the transplanted brains.

### Example 14: The ASPA iOPCs Exhibit Robust Efficacy and Preliminary Safety in Transplanted CD (Nur7) Mice

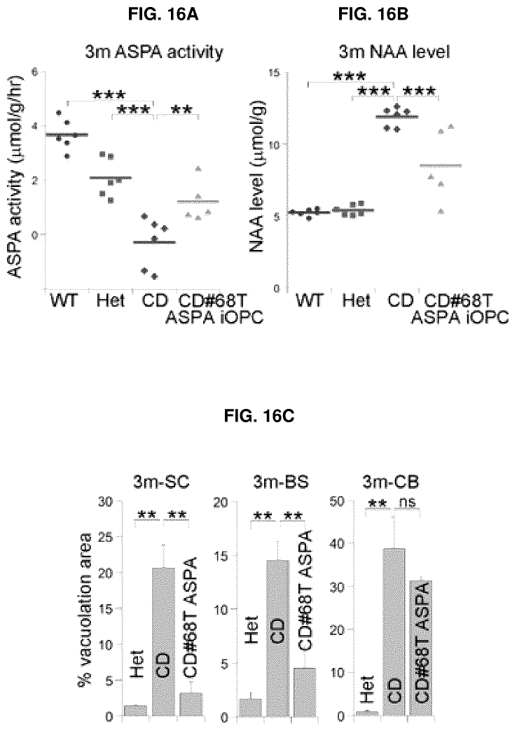

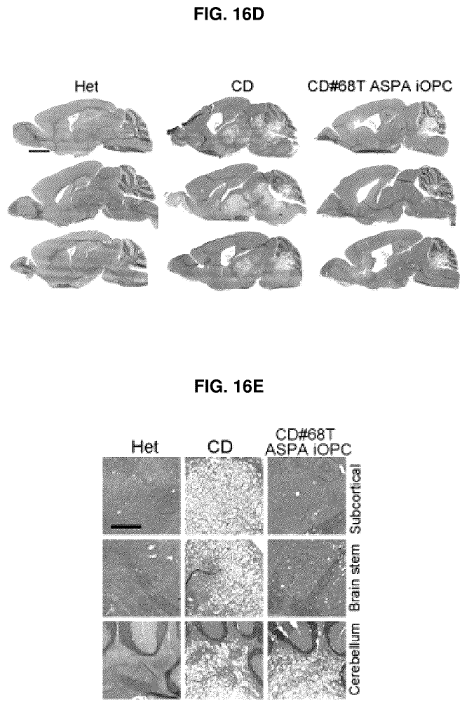

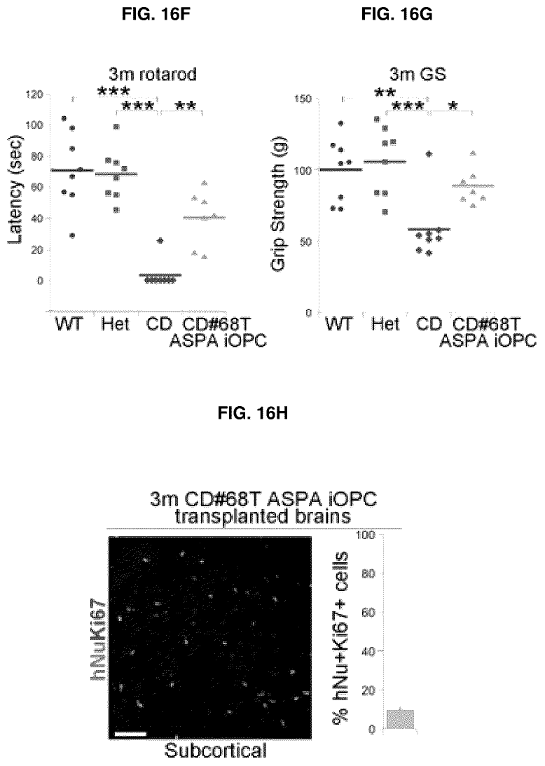

To determine the efficacy of the ASPA iOPCs, the ASPA iOPCs were transplanted into CD (Nur7) mice and the transplanted mice were evaluated three months after transplantation. Biochemically, the ASPA iOPCs were able to reconstitute ASPA enzymatic activity and reduce NAA level in the transplanted CD (Nur7) mouse brains (FIGS. 16A, 16B). The spongy degeneration was also rescued substantially in brains of the ASPA iOPC-transplanted CD (Nur7) mice, compared to the control CD (Nur7) mice (FIGS. 16C-16E). Transplantation with the ASPA iOPCs also improved myelination in CD (Nur7) mice brains as revealed by enhanced MBP staining (FIG. 11). Moreover, the motor function in the ASPA iOPC-transplanted CD (Nur7) mice was improved considerably, as revealed by increased latency on the rotarod (FIG. 16F) and enhanced grip strength (FIG. 16G), compared to the control CD (Nur7) mice. These results indicate that ASPA iOPCs have the potential to ameliorate the pathological phenotypes of CD.

No sign of tumor formation or other adverse effect was observed during three months after ASPA iOPC transplantation. Ki67 staining showed minimal number of hNu⁺Ki67⁺ cells out of total hNu⁺ cells in the ASPA iOPC-transplanted brains (FIGS. 15D, 16H). These results together demonstrate robust preclinical efficacy and preliminary safety of the ASPA iOPC cell product for CD therapy development.

All publications and patent documents cited herein are incorporated by reference.

### REFERENCES

[1] R. Matalon, K. Michals, D. Sebesta, M. Deanching, P. Gashkoff, J. Casanova, American journal of medical genetics 1988, 29, 463.
[2] a) R. M. Matalon, K. Michals-Matalon, Frontiers in bioscience: a journal and virtual library 2000, 5, D307; b) L. van Bogaert, I. Bertrand, Acta Neurol 1949, 49, 572.
[3] P. Leone, D. Shera, S. W. McPhee, J. S. Francis, E. H. Kolodny, L. T. Bilaniuk, D. J. Wang, M. Assadi, O. Goldfarb, H. W. Goldman, A. Freese, D. Young, M. J. During, R. J. Samulski, C. G. Janson, Science translational medicine 2012, 4, 165ra163.
[4] P. Leone, C. G. Janson, L. Bilaniuk, Z. Wang, F. Sorgi, L. Huang, R. Matalon, R. Kaul, Z. Zeng, A. Freese, S. W. McPhee, E. Mee, M. J. During, Annals of neurology 2000, 48, 27.
[5] a) M. Desgres, P. Menasche, Cell stem cell 2019, 25, 594; b) Y. Shi, H. Inoue, J. C. Wu, S. Yamanaka, Nature reviews. Drug discovery 2017, 16, 115.
[6] a) K. Takahashi, K. Tanabe, M. Ohnuki, M. Narita, T. Ichisaka, K. Tomoda, S. Yamanaka, Cell 2007, 131, 861; b) J. Yu, M. A. Vodyanik, K. Smuga-Otto, J. Antosiewicz-Bourget, J. L. Frane, S. Tian, J. Nie, G. A. Jonsdottir, V. Ruotti, R. Stewart, Slukvin, II, J. A. Thomson, Science 2007, 318, 1917; c) W. E. Lowry, L. Richter, R. Yachechko, A. D. Pyle, J. Tchieu, R. Sridharan, A. T. Clark, K. Plath, Proceedings of the National Academy of Sciences of the United States of America 2008, 105, 2883; d) I. H. Park, R. Zhao, J. A. West, A. Yabuuchi, H. Huo, T. A. Ince, P. H. Lerou, M. W. Lensch, G. Q. Daley, Nature 2008.
[7] a) E. Curtis, J. R. Martin, B. Gabel, N. Sidhu, T. K. Rzesiewicz, R. Mandeville, S. Van Gorp, M. Leerink, T. Tadokoro, S. Marsala, C. Jamieson, M. Marsala, J. D. Ciacci, Cell stem cell 2018, 22, 941; b) J. D. Glass, V. S. Hertzberg, N. M. Boulis, J. Riley, T. Federici, M. Polak, J. Bordeau, C. Fournier, K. Johe, T. Hazel, M. Cudkowicz, N. Atassi, L. F. Borges, S. B. Rutkove, J. Duell, P. G. Patil, S. A. Goutman, E. L. Feldman, Neurology 2016, 87, 392; c) N. Gupta, R. G. Henry, S. M. Kang, J. Strober, D. A. Lim, T. Ryan, R. Perry, J. Farrell, M. Ulman, R. Rajalingam, A. Gage, S. L. Huhn, A. J. Barkovich, D. H. Rowitch, Stem cellreports 2019, 13, 254; d) J. A. Steinbeck, L. Studer, Neuron 2015, 86, 187; e) E. S. Rosenzweig, J. H. Brock, P. Lu, H. Kumamaru, E. A. Salegio, K. Kadoya, J. L. Weber, J. J. Liang, R. Moseanko, S. Hawbecker, J. R. Huie, L. A. Havton, Y. S. Nout-Lomas, A. R. Ferguson, M. S. Beattie, J. C. Bresnahan, M. H. Tuszynski, Nature medicine 2018, 24, 484.
[8] G. H. Liu, J. Qu, K. Suzuki, E. Nivet, M. Li, N. Montserrat, F. Yi, X. Xu, S. Ruiz, W. Zhang, U. Wagner, A. Kim, B. Ren, Y. Li, A. Goebl, J. Kim, R. D. Soligalla, I. Dubova, J. Thompson, J. Yates, 3rd, C. R. Esteban, I. Sancho-Martinez, J. C. Izpisua Belmonte, Nature 2012, 491, 603.
[9] S. A. Goldman, Cell stem cell 2016, 18, 174.
[10] a) J. Piao, T. Major, G. Auyeung, E. Policarpio, J. Menon, L. Droms, P. Gutin, K. Uryu, J. Tchieu, D. Soulet, V. Tabar, Cell stem cell 2015, 16, 198; b) S. Wang, J. Bates, X. Li, S. Schanz, D. Chandler-Militello, C. Levine, N. Maherali, L. Studer, K. Hochedlinger, M. Windrem, S. A. Goldman, Cell stem cell 2013, 12, 252; c) P. Douvaras, V. Fossati, Nature protocols 2015, 10, 1143; d) L. Li, E. Tian, X. Chen, J. Chao, J. Klein, Q. Qu, G. Sun, G. Sun, Y. Huang, C. D. Warden, P. Ye, L. Feng, X. Li, Q. Cui, A. Sultan, P. Douvaras, V. Fossati, N. E. Sanjana, A. D. Riggs, Y. Shi, Cell stem cell 2018, 23, 239.
[11] P. Douvaras, J. Wang, M. Zimmer, S. Hanchuk, M. A. O'Bara, S. Sadiq, F. J. Sim, J. Goldman, V. Fossati, Stem cell reports 2014, 3, 250.
[12] a) K. Okita, Y. Matsumura, Y. Sato, A. Okada, A. Morizane, S. Okamoto, H. Hong, M. Nakagawa, K. Tanabe, K. Tezuka, T. Shibata, T. Kunisada, M. Takahashi, J. Takahashi, H. Saji, S. Yamanaka, Nature methods 2011, 8, 409; b) K. Okita, T. Yamakawa, Y. Matsumura, Y. Sato, N. Amano, A. Watanabe, N. Goshima, S. Yamanaka, Stem cells (Dayton, Ohio) 2013, 31, 458; c) C. Kime, T. A. Rand, K. N. Ivey, D. Srivastava, S. Yamanaka, K. Tomoda, Current protocols in human genetics 2015, 87, 2121.
[13] R. Kaul, G. P. Gao, M. Aloya, K. Balamurugan, A. Petrosky, K. Michals, R. Matalon, American journal of human genetics 1994, 55, 34.
[14] a) R. Matalon, K. Michals-Matalon, Advances in pediatrics 1999, 46, 493; b) R. Kaul, G. P. Gao, K. Balamurugan, R. Matalon, Nature genetics 1993, 5, 118.
[15] S. Charrier, M. Ferrand, M. Zerbato, G. Precigout, A. Viornery, S. Bucher-Laurent, S. Benkhelifa-Ziyyat, O. W. Merten, J. Perea, A. Galy, Gene Ther 2011, 18, 479.
[16] C. N. Madhavarao, J. A. Hammer, R. H. Quarles, M. A. Namboodiri, Analytical biochemistry 2002, 308, 314.
[17] M. Traka, R. L. Wollmann, S. R. Cerda, J. Dugas, B. A. Barres, B. Popko, The Journal of neuroscience: the official journal of the Society for Neuroscience 2008, 28, 11537.
[18] Y. Shinkai, G. Rathbun, K. P. Lam, E. M. Oltz, V. Stewart, M. Mendelsohn, J. Charron, M. Datta, F. Young, A. M. Stall, et al., Cell 1992, 68, 855.
[19] a) R. Matalon, P. L. Rady, K. A. Platt, H. B. Skinner, M. J. Quast, G. A. Campbell, K. Matalon, J. D. Ceci, S. K. Tyring, M. Nehls, S. Surendran, J. Wei, E. L. Ezell, S. Szucs, The journal of gene medicine 2000, 2, 165; b) N. Mersmann, D. Tkachev, R. Jelinek, P. T. Roth, W. Mobius, T. Ruhwedel, S. Ruhle, W. Weber-Fahr, A. Sartorius, M. Klugmann, PloS one 2011, 6, e20336.
[20] a) N. W. Dunham, T. S. Miya, J Am Pharm Assoc Am Pharm Assoc 1957, 46, 208; b) B. J. Jones, D. J. Roberts, J Pharm Pharmacol 1968, 20, 302.
[21] J. N. Crawley, Brain research 1999, 835, 18.
[22] F. J. Sim, C. R. McClain, S. J. Schanz, T. L. Protack, M. S. Windrem, S. A. Goldman, Nature biotechnology 2011, 29, 934.
[23] a) R. Matalon, K. Michals, R. Kaul, The Journal of pediatrics 1995, 127, 511; b) S. S. Ahmed, G. Gao, JIMD Rep 2015, 19, 11; c) H. Hoshino, M. Kubota, Pediatrics international: official journal of the Japan Pediatric Society 2014, 56, 477.
[24] C. E. Brown, D. Alizadeh, R. Starr, L. Weng, J. R. Wagner, A. Naranjo, J. R. Ostberg, M. S. Blanchard, J. Kilpatrick, J. Simpson, A. Kurien, S. J. Priceman, X. Wang, T. L. Harshbarger, M. D'Apuzzo, J. A. Ressler, M. C. Jensen, M. E. Barish, M. Chen, J. Portnow, S. J. Forman, B. Badie, The New England journal of medicine 2016, 375, 2561.
[25] X. Wang, W. C. Chang, C. W. Wong, D. Colcher, M. Sherman, J. R. Ostberg, S. J. Forman, S. R. Riddell, M. C. Jensen, Blood 2011, 118, 1255.
[26] a) J. P. Guilinger, V. Pattanayak, D. Reyon, S. Q. Tsai, J. D. Sander, J. K. Joung, D. R. Liu, Nature methods 2014, 11, 429; b) D. Hockemeyer, H. Wang, S. Kiani, C. S. Lai, Q. Gao, J. P. Cassady, G. J. Cost, L. Zhang, Y. Santiago, J. C. Miller, B. Zeitler, J. M. Cherone, X. Meng, S. J. Hinkley, E. J. Rebar, P. D. Gregory, F. D. Urnov, R. Jaenisch, Nature biotechnology 2011, 29, 731; c) C. Mussolino, R. Morbitzer, F. Lutge, N. Dannemann, T. Lahaye, T. Cathomen, Nucleic acids research 2011, 39, 9283; d) M. Boettcher, M. T. McManus, Molecular cell 2015, 58, 575.
[27] a) C. Janson, S. McPhee, L. Bilaniuk, J. Haselgrove, M. Testaiuti, A. Freese, D. J. Wang, D. Shera, P. Hurh, J. Rupin, E. Saslow, O. Goldfarb, M. Goldberg, G. Larijani, W. Sharrar, L. Liouterman, A. Camp, E. Kolodny, J. Samulski, P. Leone, Human gene therapy 2002, 13, 1391; b) R. Matalon, S. Surendran, P. L. Rady, M. J. Quast, G. A. Campbell, K. M. Matalon, S. K. Tyring, J. Wei, C. S. Peden, E. L. Ezell, N. Muzyczka, R. J. Mandel, Mol Ther 2003, 7, 580; c) S. W. McPhee, J. Francis, C. G. Janson, T. Serikawa, K. Hyland, E. O. Ong, S. S. Raghavan, A. Freese, P. Leone, Brain research. Molecular brain research 2005, 135, 112; d) S. W. McPhee, C. G. Janson, C. Li, R. J. Samulski, A. S. Camp, J. Francis, D. Shera, L. Lioutermann, M. Feely, A. Freese, P. Leone, The journal of gene medicine 2006, 8, 577; e) S. S. Ahmed, H. Li, C. Cao, E. M. Sikoglu, A. R. Denninger, Q. Su, S. Eaton, A. A. Liso Navarro, J. Xie, S. Szucs, H. Zhang, C. Moore, D. A. Kirschner, T. N. Seyfried, T. R. Flotte, R. Matalon, G. Gao, Molecular therapy: the journal of the American Society of Gene Therapy 2013, 21, 2136; f) J. S. Francis, I. Wojtas, V. Markov, S. J. Gray, T. J. McCown, R. J. Samulski, L. T. Bilaniuk, D. J. Wang, D. C. De Vivo, C. G. Janson, P. Leone, Neurobiology of disease 2016, 96, 323; g) S. S. Ahmed, S. A. Schattgen, A. E. Frakes, E. M. Sikoglu, Q. Su, J. Li, T. G. Hampton, A. R. Denninger, D. A. Kirschner, B. Kaspar, R. Matalon, G. Gao, Molecular therapy: the journal of the American Society of Gene Therapy 2016, 24, 1030; h) D. J. Gessler, D. Li, H. Xu, Q. Su, J. Sanmiguel, S. Tuncer, C. Moore, J. King, R. Matalon, G. Gao, JCI insight 2017, 2, e90807; i) G. von Jonquieres, Z. H. T. Spencer, B. D. Rowlands, C. B. Klugmann, A. Bongers, A. E. Harasta, K. E. Parley, J. Cederholm, O. Teahan, R. Pickford, F. Delerue, L. M. Ittner, D. Frohlich, C. A. McLean, A. S. Don, M. Schneider, G. D. Housley, C. D. Rae, M. Klugmann, Acta neuropathologica 2018, 135, 95; j) M. H. Baslow, Journal of inherited metabolic disease 2017, DOI: 10.1007/s10545-017-0038-2; k) S. Starling, Nature reviews. Neurology 2018, 14, 4.
[28] a) V. Hull, Y. Wang, T. Burns, S. Zhang, S. Sternbach, J. McDonough, F. Guo, D. Pleasure, Annals of neurology 2020, 87, 480; b) P. Bannerman, F. Guo, O. Chechneva, T. Burns, X. Zhu, Y. Wang, B. Kim, N. K. Singhal, J. A. McDonough, D. Pleasure, Mol Ther 2018, 26, 793.
[29] P. Colella, G. Ronzitti, F. Mingozzi, Mol Ther Methods Clin Dev 2018, 8, 87.
[30] N. Gupta, R. G. Henry, J. Strober, S. M. Kang, D. A. Lim, M. Bucci, E. Caverzasi, L. Gaetano, M. L. Mandelli, T. Ryan, R. Perry, J. Farrell, R. J. Jeremy, M. Ulman, S. L. Huhn, A. J. Barkovich, D. H. Rowitch, Science translational medicine 2012, 4, 155ra137.
[31] S. A. Goldman, M. Nedergaard, M. S. Windrem, Science (New York, N. Y 2012, 338, 491.
[32] a) M. S. Windrem, S. J. Schanz, M. Guo, G. F. Tian, V. Washco, N. Stanwood, M. Rasband, N. S. Roy, M. Nedergaard, L. A. Havton, S. Wang, S. A. Goldman, Cell stem cell 2008, 2, 553; b) M. S. Windrem, M. C. Nunes, W. K. Rashbaum, T. H. Schwartz, R. A. Goodman, G. McKhann, 2nd, N. S. Roy, S. A. Goldman, Nature medicine 2004, 10, 93.
[33] M. H. Baslow, Neurochemical research 2003, 28, 941.
[34] M. Mandai, Y. Kurimoto, M. Takahashi, The New England journal of medicine 2017, 377, 792.
[35] J. I. Pearl, L. S. Kean, M. M. Davis, J. C. Wu, Science translational medicine 2012, 4, 164ps25.
[36] M. Umekage, Y. Sato, N. Takasu, Inflamm Regen 2019, 39, 17.
[37] a) G. G. Gornalusse, R. K. Hirata, S. E. Funk, L. Riolobos, V. S. Lopes, G. Manske, D. Prunkard, A. G. Colunga, L. A. Hanafi, D. O. Clegg, C. Turtle, D. W. Russell, Nature biotechnology 2017, 35, 765; b) Z. Rong, M. Wang, Z. Hu, M. Stradner, S. Zhu, H. Kong, H. Yi, A. Goldrath, Y. G. Yang, Y. Xu, X. Fu, Cell stem cell 2014, 14, 121; c) T. Deuse, X. Hu, A. Gravina, D. Wang, G. Tediashvili, C. De, W. O. Thayer, A. Wahl, J. V. Garcia, H. Reichenspurner, M. M. Davis, L. L. Lanier, S. Schrepfer, Nature biotechnology 2019, 37, 252; d) H. Xu, B. Wang, M. Ono, A. Kagita, K. Fujii, N. Sasakawa, T. Ueda, P. Gee, M. Nishikawa, M. Nomura, F. Kitaoka, T. Takahashi, K. Okita, Y. Yoshida, S. Kaneko, A. Hotta, Cell stem cell 2019, 24, 566.
[38] H. H. Kuo, X. Gao, J. M. DeKeyser, K. A. Fetterman, E. A. Pinheiro, C. J. Weddle, H. Fonoudi, M. V. Orman, M. Romero-Tejeda, M. Jouni, M. Blancard, T. Magdy, C. L. Epting, A. L. George, Jr., P. W. Burridge, Stem cell reports 2020, 14, 256.
[39] a) P. Y. Yam, S. Li, J. Wu, J. Hu, J. A. Zaia, J. K. Yee, Mol Ther 2002, 5, 479; b) D. L. DiGiusto, A. Krishnan, L. Li, H. Li, S. Li, A. Rao, S. Mi, P. Yam, S. Stinson, M. Kalos, J. Alvarnas, S. F. Lacey, J. K. Yee, M. Li, L. Couture, D. Hsu, S. J. Forman, J. J. Rossi, J. A. Zaia, Science translational medicine 2010, 2, 36ra43.
[40] a) Q. Qu, G. Sun, W. Li, S. Yang, P. Ye, C. Zhao, R. T. Yu, F. H. Gage, R. M. Evans, Y. Shi, Nature cell biology 2010, 12, 31; b) Q. Cui, S. Yang, P. Ye, E. Tian, G. Sun, J. Zhou, G. Sun, X. Liu, C. Chen, K. Murai, C. Zhao, K. T. Azizian, L. Yang, C. Warden, X. Wu, M. D'Apuzzo, C. Brown, B. Badie, L. Peng, A. D. Riggs, J. J. Rossi, Y. Shi, Nature communications 2016, 7, 10637; c) Y. Shi, C. D. Lie, P. Taupin, K. Nakashima, J. Ray, R. T. Yu, F. H. Gage, R. M. Evans, Nature 2004, 427, 78.
[41] K. Qian, C. T. Huang, H. Chen, L. W. t. Blackbourn, Y. Chen, J. Cao, L. Yao, C. Sauvey, Z. Du, S. C. Zhang, Stem cells 2014, 32, 1230.
[42] H. Li, R. Durbin, Bioinformatics 2009, 25, 1754.
[43] A. McKenna, M. Hanna, E. Banks, A. Sivachenko, K. Cibulskis, A. Kernytsky, K. Garimella, D. Altshuler, S. Gabriel, M. Daly, M. A. Depristo, Genome research 2010, 20, 1297.
[44] J. Grau, J. Boch, S. Posch, Bioinformatics 2013, 29, 2931.
[45] A. Prokesch, H. J. Pelzmann, A. R. Pessentheiner, K. Huber, C. T. Madreiter-Sokolowski, A. Drougard, M. Schittmayer, D. Kolb, C. Magnes, G. Trausinger, W. F. Graier, R. Birner-Gruenberger, J. A. Pospisilik, J. G. Bogner-Strauss, Scientific reports 2016, 6, 23723.
[46] S. Charrier, M. Ferrand, M. Zerbato, G. Précigout, A. Viornery, S. Bucher-Laurent, S. Benkhelifa-Ziyyat, O. W. Merten, J. Perea, A. Galy, 2011, 18, 479.
[47] N. Uchida, K. Chen, M. Dohse, K. D. Hansen, J. Dean, J. R. Buser, A. Riddle, D. J. Beardsley, Y. Wan, X. Gong, T. Nguyen, B. J. Cummings, A. J. Anderson, S. J. Tamaki, A. Tsukamoto, I. L. Weissman, S. G. Matsumoto, L. S. Sherman, C. D. Kroenke, S. A. Back, Science translational medicine 2012, 4, 155ra136.
[48] J. E. Le Belle, N. G. Harris, S. R. Williams, K. K. Bhakoo, NMR in biomedicine 2002, 15, 37.
[49] J. B. West, Z. Fu, T. J. Deerinck, M. R. Mackey, J. T. Obayashi, M. H. Ellisman, Respiratory physiology & neurobiology 2010, 170, 202.
[50] J. Le Coq, H. An, C. Lebrilla, R. Viola, Biochemistry 2006, 45: 5878-5884.

1-32. (canceled)

33. A method for treating Canavan disease in a subject, comprising:
reprogramming or converting somatic cells isolated from the subject into induced pluripotent stem cells (iPSCs);
differentiating the iPSCs into neural precursor cells;
introducing a functional ASPA gene having a R132G mutation (R132G ASPA) into the neural precursor cells to obtain genetically corrected neural precursor cells which express the R132G ASPA; and
transplanting the genetically corrected neural precursor cells into the brain of the subject.

34. The method of claim 33, wherein the neural precursor cells include neural progenitor cells (NPCs), glial progenitor cells and oligodendroglial progenitor cells (OPCs).

35. (canceled)

36. The method of claim 33, wherein the R132G ASPA comprises one or more mutations outside of the catalytic center.

37. (canceled)

38. A method of producing ASPA neural precursor cells, comprising:
reprogramming or converting somatic cells isolated from a subject suffering from Canavan disease into induced pluripotent stem cells (iPSCs);
differentiating the iPSCs into neural precursor cells; and
introducing a functional ASPA gene having a R132G mutation (R132G ASPA) in the neural precursor cells to obtain genetically corrected neural precursor cells which express R132G ASPA.

39. The method of claim 38, wherein the neural precursor cells include NPCs, glial progenitor cells and OPCs.

40. (canceled)

41. The method of claim 38, wherein the R132G ASPA comprises one or more mutations outside of the catalytic center.

42. (canceled)

43. Neural precursor cells which express an exogenous functional ASPA gene produced by a process comprising the steps of:
reprogramming or converting somatic cells isolated from a subject suffering from Canavan disease into induced pluripotent stem cells (iPSCs),
differentiating the iPSCs into neural precursor cells; and
introducing a functional ASPA gene having a R132G mutation (R132G ASPA) in the neural precursor cells to obtain genetically corrected neural precursor cells which express R132G ASPA.

44. (canceled)

45. The neural precursor cells of claim 43, wherein the R132G ASPA comprises one or more mutations outside of the catalytic center.

46. (canceled)

47. The method of claim 33, wherein the reprogramming is carried out in the presence of one or more reprogramming factors comprising OCT4, SOX2, KLF4, LIN28 and MYC.

48. The method of claim 33, wherein the reprogramming is carried out via episomal reprogramming or viral transduction.

49. The method of claim 33, wherein the somatic cells are fibroblasts, blood cells, urinary cells, adipocytes keratinocytes, or dental pulp cells.

50. The method of claim 33, wherein the functional ASPA gene is introduced by transducing the neural precursor cells with a vector comprising the functional ASPA gene or by correcting the ASPA gene mutation using gene editing technology.

51. The method of claim 50, wherein the gene editing includes CRISPR or TALEN-mediated genetic engineering.

52. The method of claim 50, wherein the vector is lentivirus.

53. The method of claim 38, wherein the reprogramming is carried out in the presence of one or more reprogramming factors comprising OCT4, SOX2, KLF4, LIN28 and MYC.

54. The method of claim 38, wherein the reprogramming is carried out via episomal reprogramming or viral transduction.

55. The method of claim 38, wherein the somatic cells are fibroblasts, blood cells, urinary cells, adipocytes keratinocytes, or dental pulp cells.

56. The method of claim 38, wherein the functional ASPA gene is introduced by transducing the neural precursor cells with a vector comprising the functional ASPA gene or by correcting the ASPA gene mutation using gene editing technology.

57. The method of claim 56, wherein the gene editing includes CRISPR or TALEN-mediated genetic engineering.

58. The method of claim 56, wherein the vector is lentivirus.
